(19) United States
(12) Patent Application Publication
(10) Pub. No.: US 2024/0180461 A1
(43) Pub. Date: Jun. 6, 2024

(54) ELECTROCHEMICAL SENSOR FOR THE MEASUREMENT OF GLUCOSE CONCENTRATION

(71) Applicant: Arkansas State University – Jonesboro, State University, AR (US)

(72) Inventors: Anahita Izadyar, State University, AR (US); Elizabeth E. Hood, Jonesboro, AR (US)

(21) Appl. No.: 18/514,824

(22) Filed: Nov. 20, 2023

Related U.S. Application Data
(63) Continuation-in-part of application No. PCT/US22/30096, filed on May 19, 2022.
(60) Provisional application No. 63/529,602, filed on Jul. 28, 2023.
(60) Provisional application No. 63/190,688, filed on May 19, 2021.
(60) Provisional application No. 63/235,488, filed on Aug. 20, 2021.

(51) Int. Cl. A61B 5/1486; A61B 5/00; A61B 5/145; A61B 5/1477

(52) U.S. Cl. CPC A61B 5/1486; A61B 5/14532; A61B 5/1477; A61B 5/4836; A61B 2560/0468; A61B 2562/028

(57) ABSTRACT

The present invention is directed to an electrochemical sensor for the measurement of glucose concentration comprising one or more electrodes, a coating that surrounds the one or more electrodes, and two or more enzymes distributed within the coating, wherein the two or more enzymes comprises peroxidase and glucose oxidase.

This application claims priority to U.S. Provisional Application No. 63/529,602, filed on Jul. 28, 2023, and is a continuation in part of International Application No. PCT/US2022/30096, filed in the United States receiving office on May 19, 2022, the entirety of which is incorporated herein by reference, and which in turn derives priority from U.S. Provisional Application No. 63/190,688, filed on May 19, 2021, and U.S. Provisional Application No. 63/235,488, filed on Aug. 20, 2021, the entire contents of each of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. 2021-70001-34524 awarded by the USDA, NIFA. The government has certain rights in the invention.

### FIELD OF THE INVENTION

This invention is directed to an electrochemical sensor for the measurement of glucose concentration comprising one or more electrodes, a coating that surrounds the one or more electrodes, and two or more enzymes distributed within the coating, wherein the two or more enzymes comprises peroxidase and glucose oxidase.

### BACKGROUND OF THE INVENTION

In our daily lives, measuring the amount of sugar in food and drink or for healthcare is crucial. [1-3] By monitoring blood glucose for diabetic people on a regular basis, a healthy life can be ensured. Diabetes is one of the most common chronic diseases, and is due to an imbalance in the body's glucose levels. Diabetes mellitus (DM) is one of the leading causes of death in any country, and malnutrition makes diabetes one of the most common chronic diseases. Diabetes caused 1.5 million deaths in 2012. Excessive blood glucose increases the risk of cardiovascular and other diseases by another 2.2 million. As of 2019, an estimated 463 million people worldwide will suffer from diabetes, and without wishing to be bound by theory this number can reach 700 million by 2045. [4-6] Diabetics suffer from many disorders in their body and thus their treatment is more painful as they address multiple conditions.

### SUMMARY OF THE INVENTION

An aspect of the invention is directed to an electrochemical sensor for the measurement of glucose concentration. In embodiments, the electrochemical sensor comprises one or more electrodes, a coating that surrounds the one or more electrodes, and one or more enzymes distributed within the coating, wherein the one or more enzymes comprises peroxidase, glucose oxidase, or a combination thereof, and wherein the one or more electrodes comprises a screen-printed electrode.

In embodiments, the coating is deposited on the surface of the one or more electrodes.

In embodiments, the peroxidase comprises manganese peroxidase. For example, the manganese peroxidase comprises fungal manganese peroxidase. For example, the fungus comprises Phanerochaete chrysosporium.

In embodiments, the peroxidase is recombinantly produced.

In embodiments, the peroxidase is recombinantly produced in a plant or plant cell.

In embodiments, the coating comprises bovine serum albumin (BSA), glutaraldehyde, or a combination thereof.

In embodiments, the coating comprises manganese peroxidase, polythiophene, glucose oxidase, bovine serum albumin (BSA), and glutaraldehyde.

In embodiments, the coating comprises about 0.1 mg to about 20 mg PPMP, about 0.1 mg to about 15 mg Gox, about 0.5 μL to about 50 μL of 45 μM BSA, and about 5 μL to about 50 μL glutaraldehyde (2.5%).

In embodiments, the coating further comprises a conductive polymer, an anti-fouling polymer, or a combination thereof. For example, the conductive polymer comprises a perfluorosulphonic acid polymer, 3,4-ethylene dioxy thiophene (EDOT), polyaniline (PANI), pyrrole, polypyrrole, polythiophene, polypyrrole, polythiophene, or Poly(3,4-ethylenedioxythiophene)-poly(styrenesulfonate), (PEDOT:PSS). For example, the conductive polymer is sulfonated tetrafluoroethylene (Nafion™).

In embodiments, the conductive polymer is deposited on the coating that surrounds the one or more electrodes.

In embodiments, the anti-fouling polymer comprises poly(ethylene glycol) (PEG), zwitterionic polymers, poly(hydroxy functional acrylates), poly(2-oxazoline)s, poly(vinylpyrrolidone), poly(glycerol), peptides and peptoids.

In embodiments, the coating further comprises a nanoparticle. For example, the nanoparticle layer can be between the one or more electrode and the coating. For example, the nanoparticle layer can be deposited on the one or more electrodes. For example, the nanoparticle comprises graphene, carbon nanotubes, borophene, gold, or platinum.

In embodiments, the coating has a thickness of about 100 μm to about 1 mm. For example, the coating is less than about 1.5 mm thick.

In embodiments, the coating at least partially surrounds the working electrode or surrounds the working electrode.

In embodiments, the one or more electrodes comprises a counter electrode, a working electrode, a reference electrode, or a combination thereof. For example, the counter electrode comprises platinum. For example, the working electrode comprises gold or carbon. For example, the reference electrode comprises silver (Ag), silver/silver chloride (Ag/AgCl) or saturated calomel electrode (SCE).

In embodiments, the electrochemical sensor is an amperometric sensor and/or a voltammetric sensor.

In embodiments, the electrochemical sensor can detect glucose in a fluid sample. For example, the fluid sample comprises whole blood, serum, plasma. For example, the fluid sample comprises a consumable.

In embodiments, the electrochemical sensor can detect between about 20.0 μM and about 15.0 mM glucose.

In embodiments, the sensor has a lower detection limit of about 2.9 μM.

In embodiments, the coating allows partitioning of glucose directly from a fluid sample, partitions a biocompatible interface between the sensor and the fluid sample, prevents electrode fouling, and/or provides selectivity for glucose.

In embodiments, the electrodes are present in a microfluidic device in communication with a microfluid channel.

In embodiments, the one or more electrodes are immobilized on a support member.

In embodiments, the sensor is miniaturized.

Aspects of the invention are further drawn towards a microfluid device comprising the electrochemical sensor as described herein.

Further, aspects of the invention are drawn towards a method for electrochemical detection of glucose in a sample. In embodiments, the method comprises exposing a fluid sample obtained from a subject to the electrochemical sensor as described herein, and detecting the current generated from the oxidation of H₂O₂ during said exposing, wherein current corresponds to the concentration of glucose in the fluid sample.

In embodiments, the fluid sample comprises whole blood, plasma, or serum. In embodiments, the fluid sample comprises a consumable.

In embodiments, said detecting is repeated periodically.

Aspects of the invention are also drawn towards a method of treating a subject afflicted with or at risk of diabetes. In embodiments, the method comprises exposing a fluid sample obtained from a subject to the electrochemical sensor as described herein, detecting the current generated from the oxidation of H₂O₂ during said exposing, wherein current corresponds to the concentration of glucose in the fluid sample, and treating the subject for diabetes if the subject has a fasting blood glucose concentration is greater than 100 mg/dl.

For example, the diabetes comprises diabetes mellitus.

Further, aspects of the invention are drawn towards a method of diagnosing a subject with or at risk of diabetes. In embodiments, the method comprises exposing a fluid sample obtained from a subject to the electrochemical sensor as described herein, detecting the current generated from the oxidation of H₂O₂ during said exposing, wherein current corresponds to the concentration of glucose in the fluid sample, and diagnosing the subject as having or at risk of having diabetes if the subject has a fasting blood glucose concentration is greater than 100 mg/dl.

In embodiments, diabetes comprises diabetes mellitus.

Aspects of the invention are also drawn towards a method of monitoring a subject having or at risk of having diabetes. In embodiments, the method comprises exposing a fluid sample obtained from a subject to an electrochemical sensor as described herein, detecting the current generated from the oxidation of H₂O₂ during said exposing, wherein current correlates to the concentration of glucose in the fluid sample, thereby monitoring the subject having or at risk of having diabetes.

Aspects of the invention are drawn towards a kit comprising an electrochemical sensor as described herein.

Further, aspects of the invention are drawn towards an electrochemical sensor for the measurement of glucose concentration comprising one or more electrodes, a coating that surrounds the one or more electrodes, and one or more enzymes distributed within the coating, wherein the one or more enzymes comprises manganese peroxidase and glucose oxidase immobilized onto one or more of the electrodes by polymerizing aniline, and, optionally, wherein Nafion™ polymer is deposited on the coated one or more electrodes.

Aspects of the invention are also drawn towards an electrochemical sensor for the measurement of glucose concentration comprising one or more electrodes, a coating that surrounds the one or more electrodes, and one or more enzymes distributed within the coating, wherein the one or more enzymes comprises manganese peroxidase and glucose oxidase immobilized onto one or more of the electrodes by polymerizing aniline, and, optionally, wherein the one or more electrodes comprise a gold nanoparticle layer between the electrode and the coating.

Other objects and advantages of this invention will become readily apparent from the ensuing description.

### BRIEF DESCRIPTION OF THE FIGURES

FIG. 1 shows representations of panel A) Glucose biosensor membrane on the gold electrode surface (yellow), the enzyme-based layer modified with PPMP, BSA, Gox, and Nafion™ and the SEM image. Panel B) Amperometric responses of the glucose biosensor.

FIG. 2 shows panel A) Background-subtracted linear sweep voltammograms for a Nafion™/PPMP-GOx-BSA/Au electrode, in the oxygen saturated (pH 7.0)/0.1 mM Mn (CH₃COO)₂ solution in the range of 0.10-15.0 mM of Glucose vs Ag/AgCl (scan rate of 0.05 Vs⁻¹). Panel (b) Calibration plot of background-subtracted peak current versus glucose concentration (circles). Solid line is best-fit.

FIG. 3 shows panel A) Background-subtracted linear sweep voltammograms for a PPMP-GOx-BSA/Au electrode, in the oxygen saturated (pH 7.0)/0.1 mM Mn (CH₃COO)₂ solution in the range of 0.08-6.50 mM of glucose vs Ag/AgCl (scan rate of 0.05 Vs⁻¹). Panel B) shows calibration plot of background-subtracted peak current versus glucose concentration (circles). Solid line is best-fit.

FIG. 4 shows panel A) Amperometric response of the Nafion™/PPMP-GOx-BSA/Au electrode, in the oxygen saturated (pH 7.0)/0.1 mM Mn (CH₃COO)₂ solution in the range of 0.02-15.0 mM of glucose (scan rate of 0.05 Vs⁻¹). Applied potential: 0.84 V vs Ag/AgCl. (b) Corresponding calibration plot shows the dependence of current response with the concentration of glucose.

FIG. 5 shows SEM images of the Panel (a) PPMP-GOx-BSA/Au membrane and (b) Nafion™/PPMP-GOxBSA/Au membrane. Both membranes were spin coated with 15 μL of a solution containing PPMP, GOx, BSA, and glutaraldehyde. Panel (b) An additional 7 μL of 0.05% Nafion™ was also spincoated on the electrode.

FIG. 6 shows (a) Amperometric response of Nafion™/PPMP-GOx-BSA/Au electrode, in the oxygen saturated (pH 7.0) 0.1 mM Mn (CH₃COO)₂ solution in the range of 0.02-14.0 mM of glucose containing 10% diet green tea with citrus (scan rate of 0.05 Vs⁻¹). Applied potential: 0.84 V vs Ag/AgCl (b) Corresponding calibration plot shows the dependence of current response with the concentration of glucose.

FIG. 7 shows representative graphs of data.

FIG. 8 shows a representative graph of current vs. potential.

FIG. 9 shows representative graphs of data.

FIG. 10 shows representative graphs of data.

FIG. 11 shows a schematic of the Gox-HRP reaction.

FIG. 12 shows SEM images of A) PPMP enzyme in Na-tartrate buffer pH (4.5) and Panel B) Na-tartrate buffer pH (4.5).

FIG. 13 shows a reaction scheme for glucose detection at the Au-modified PPMP enzyme electrode.

FIG. 14 shows representative graphs of panel A) Background-subtracted linear sweep voltammograms for a PPMP/Au electrode, in the oxygenated PBS solution in the range of 0.02-6.00 mM of glucose/0.1 mM Mn²⁺ vs. Ag/AgCl (scan rate of 0.05 Vs⁻¹) and Panel B) Calibration plot of background-subtracted peak current versus glucose concentration (circles). Solid line is best-fit.

FIG. 15 shows amperometric response of PPMP/Au modified electrode in the oxygenated PBS stirred solution by successive addition of glucose.

FIG. 16 shows cyclic voltammograms of reduction of boron nanoparticles electrodeposited on the gold (Au) electrode of 40 mg of boron nanoparticle (BN) distributed in 25 ml of dimethyl formamide (DMF) solvent, with sixty cycles (red), overlay with 25 ml of dimethyl formamide (DMF) background (blue).

FIG. 17 shows cyclic voltammograms of BN/Au modified electrodes in 5 Mm FC (red) nanoparticle boron electrodeposited on the Au electrode (green) bare Au electrode, (brown) nanoparticle boron spin coated on the surface of Au electrode, and blue (20 μL boron nanoparticles solution drop cast on the Au electrode surface vs Ag/AgCl as reference electrode. Scan rate 0.1 V/s).

FIG. 18 shows a graph of Electropolymerization CV at 25 mV/s. Attempt to electropolymerize pyrrole at a 1.5 mg/mL of monomer concentration in 1.6 mg/mL PPMP, and 10.2 mg/mL GOx.

FIG. 19 shows graphs of Panel A) background-subtracted linear sweep voltammograms of a PPMP/Au electrode in the oxygenated PBS solution in the range of 0.02-6.00 mM of glucose/0.1 mM Mn²⁺ vs Ag/AgCl (scan rate of 0.05 Vs⁻¹) and Panel B) Calibration plot of background-subtracted peak current versus glucose concentration (circles). Solid line is best-fit.

FIG. 20 shows a schematic of H₂O₂ undergoing a reaction with PPMP adapted from Izadyar A., Tran U., and Hood E. E. ACS Sustainable Chem. Eng. 2019, 7, 19343-19441.

FIG. 21 shows graphs of electrochemistry of the PPMP/Au electrode applying Cyclic Voltammetry (CV) and Linear Sweep Voltammetry (LSV).

FIG. 22 shows graphs of amperometric responses of the PPMP/Au electrode to H₂O₂.

FIG. 23 shows graphs of representative data for selectivity evaluation of the PPMP Biosensor in Orange juice adapted from Izadyar A., Tran U., and Hood E. E. ACS Sustainable Chemistry & Engineering. 2019, 7, 19434-19441.

FIG. 24 shows a schematic of reactions.

FIG. 25 shows a graphic of cost comparison of PPMP and HRP. *D. R. Walwyn, S. M. Huddy, E. P. Rybicki, Appl Biochem Biotechnol 175 (2015)841-854.

FIG. 26 shows graphs of LSV of the Nafion/PPMP-Gox-BSA/Au Electrode.

FIG. 27 shows graphs of amperometric responses of the Nafion™/PPMP-Gox-BSA/Au Electrode to detect glucose.

FIG. 28 shows graphs of representative data indicating the selectivity of the Nafion/PPMP-Gox-BSA/Au electrode.

FIG. 29 shows SEM image panel A) PPMP-Gox-BSA/Au and panel B) Nafion™/PPMP-Gox-BSA/Au.

FIG. 30 shows a graphic of comparisons of glucose determination with differently modified electrodes 1. H. Yang, C. Gong. L. Miao, F. A. Xu, Int. J. Electrochem. Sci, 12 (2017) 4958-4969. 2. K. S. B. Dinesh, S. Devi, U. M. Krishnan, ACS Appl. BioMater., 2 (2019) 1740-1750. 3. N. German, A. Ramanavicius, A. Ramanaviciene, Electroanalysis, 29 (2017) 1267-1277. 4. B. K. Shrestha, R. Ahmad, H. M. Mousa, I. G. Kim, J. I. Kim, Sci, 482 (2016) 39-47. 5. S. Xu, H. Qi, S. Zhou, X. Zhang, C. Zhang, Microchim Acta 181 (2014) 535-541.

FIG. 31 shows graphs of electrochemical data of the biosensor.

FIG. 32 shows graphs of electrochemical data of the biosensor.

FIG. 33 shows graphs of electrochemical data of the biosensor.

FIG. 34 shows a graphic of H₂O₂ reaction with PPMP.

FIG. 35 shows graphs of LSV, amperometric responses of the PPMP/Au electrode to H₂O₂.

FIG. 36 shows graphs of representative data for selectivity evaluation of the PPMP Biosensor in Orange juice adapted from Izadyar A., Tran U., and Hood E. E. ACS Sustainable Chemistry & Engineering. 2019, 7, 19434-19441.

FIG. 37 shows a graphic of GMP at scale cost comparison of PPMP and HRP. *D. R. Walwyn, S. M. Huddy, E. P. Rybicki, Appl Biochem Biotechnol 175 (2015)₈₄₁-854.

FIG. 38 shows graphs of LSV of the Nafion™/PPMP-Gox-BSA/Au Electrode to detect glucose.

FIG. 39 shows graphs of representative data indicating the selectivity of the Nafion/PPMP-Gox-BSA/Au electrode adapted from Izadyar A., Rodriguez K. A., Van, M. N., Tran U., and Hood E. E. Journal of Electroanalytical Chemistry. 2021, 895, 115387.

FIG. 40 shows graphs of LVS, gold electrode, electropolymerized with aniline, GOX, PPMP, BSA.

FIG. 41 shows graphs of cyclic voltammogram with nanoparticle.

FIG. 42 shows graphs of LVS gold electrode, electropolymerized with Au, nanoparticle (AuNPs), aniline, GOX, PPMP, BSA.

FIG. 43 shows graphs of electrochemical data.

FIG. 44 shows a graphic of H₂O₂ reaction with PPMP.

FIG. 45 shows *GMP at scale Cost comparison of PPMP and HRP. *D. R. Walwyn, S. M. Huddy, E. P. Rybicki, Appl Biochem Biotechnol 175 (2015) 841-854.

FIG. 46 shows a picture of an experimental setup of a 3-electrode cell.

FIG. 47 shows graphs of Linear Sweep Voltammetry (LSV), Responses of the PPMP(Na-tartrate buffer pH 4.5)/Au electrode to H₂O₂.

FIG. 48 shows graphs of Amperometric Responses of the PPMP(Na-tartrate buffer pH 4.5)/Au electrode to H₂O_{2.}

FIG. 49 shows Linear Sweep Voltammetry (LSV), Selectivity Evaluation of the PPMP Biosensor in Orange Juice adapted from Izadyar A., et al; ACS Sustainable Chemistry & Engineering. 2019, 7, 19434-19441.

FIG. 50 shows Amperometric, Selectivity Evaluation of the PPMP Biosensor in Orange Juice adapted from Izadyar A., et al; ACS Sustainable Chemistry & Engineering. 2019, 7. 19434-19441.

FIG. 51 shows Linear Sweep Voltammetry (LSV), Responses of the Nafion/PPMP-GOx-BSA/Au Electrode to detect glucose adapted from Izadyar A. et al: Journal of Electroanalytical Chemistry. 2021, 895, 115387.

FIG. 52 shows Amperometric, Responses of the Nafion™/PPMP-GOx-BSA/Au Electrode to detect glucose.

FIG. 53 shows Amperometric, Selectivity of the Nafion™/PPMP-GOx-BSA/Au Electrode adapted from Izadyar A. et al: Journal of Electroanalytical Chemistry. 2021, 895, 115387.

FIG. 54 shows a graphic of conductive polymer nanocomposite-based biosensors.

FIG. 55 shows graphics of embodiments of the disclosure.

FIG. 56 shows a graphic and graphs of Gold electrode. Electropolymerized with 15 cycles of Au, nanoparticle, Aniline, GOX, PPMP, BSA in PBS (pH 7.0).

FIG. 57 shows a graphic of comparisons of LSV of glucose sensor data using PPMP.

FIG. 58 shows bienzymatic nanocomposites biosensors to measure glucose.

FIG. 59 shows a graph of cyclic voltammetric sweep of 20 cycles with potential ranging from −0.25 to 0.45 V/Ag/AgCl, at a scan rate of 0.05 Vs⁻¹.

FIG. 60 shows panel (A) Background-subtracted of LSVs for a PANI-GOX-PPMP-AuNPs/Au electrode, in the oxygen saturated PBS/0.1 mM Mn(CH₃COO)₂ solution in the range of 0.005 to 16.0 mM of glucose vs Ag/AgCl (Scan rate of 0.05 Vs⁻¹). Panel (B) Calibration plot of background-subtracted peak current versus glucose concentration (R²=0.9990).

FIG. 61 shows a graphic of embodiments of the electrode coating.

FIG. 62 shows panel (a) Non-background-subtracted of LSVs for a PANI-GNPs-GOx-PPMP/GME, in the oxygen saturated PBS/0.1 mM Mn(CH₃COO)₂ solution in the range of 0.02 to 16.0 mM of glucose vs Ag/AgCl (Scan rate of 0.05 Vs⁻¹). Panel (b) Calibration plot of non-background-subtracted peak current versus glucose concentration (R²=0.9991).

FIG. 63 shows panel (a) Non-background-subtracted of CVs for a PANI-GNPs-GOx-PPMP/GME, in the oxygen saturated PBS/0.1 mM Mn(CH₃COO)₂ solution in the range of 0.01 to 16.0 mM of glucose vs Ag/AgCl (Scan rate of 0.05 Vs⁻¹). Panel (b) Calibration plot of non-background-subtracted peak current versus glucose concentration (R²=0.9990).

FIG. 64 shows panel (a) Chronoamperograms at the PANI-GNPs-GOx-PPMP/GME in the oxygen saturated PBS/0.1 mM Mn(CH₃COO)₂ containing different concentrations of glucose from 0.005-16.0 mM at 0.73V potential versus Ag/AgCl (Scan rate of 0.05 Vs⁻¹). Panel (b) Calibration curve for glucose peak current versus glucose concentration (R²=0.9991).

FIG. 65 shows Panel (a) Chronoamperograms at the PANI-GNPs-GOx-PPMP/GME in the oxygen saturated PBS/0.1 mM Mn(CH₃COO)₂ containing different concentrations of glucose from 0.005-16.0 mM in 1.0 mM ascorbic acid at 0.73V potential versus Ag/AgCl (Scan rate of 0.05 Vs⁻¹). Panel (b) Calibration curve for glucose peak current versus glucose concentration (R²=0.9995).

FIG. 66 shows Panel (a) Non-background-subtracted LSV plots of the PANI-GNPs-GOx-PPMP/GME, at different scan rates of 0.01, 0.03, 0.05, 0.07, 0.10, 0.20 Vs⁻¹ vs Ag/AgCl in 1.0 mM glucose in PBS pH 7/0.1 mM Mn(CH₃COO)₂ in an oxygen saturated solution: Panel (B) Linear relationship between the oxidation peak current and the square root of the scan rate (R²=0.9994).

FIG. 67 shows panel (a) Non-background-subtracted of LSVs for a PANI-GNPs-GOx-PPMP/GME, in the oxygen saturated PBS/0.1 mM Mn(CH₃COO)₂ solution in the range of 0.005 to 16.0 mM of glucose contain 1 mM ascorbic acid vs Ag/AgCl (Scan rate of 0.05 Vs⁻¹). Panel (b) Calibration plot of non-background-subtracted peak current versus glucose concentration (R²=0.9993).

FIG. 68 shows panel (a) Non-background-subtracted of LSVs for a PANI-GNPs-GOx-PPMP/GME, in the oxygen saturated PBS/0.1 mM Mn(CH₃COO)₂ solution in the range of 0.005 to 16.0 mM of glucose contain 1 mM Caffeine vs Ag/AgCl (Scan rate of 0.05 Vs⁻¹). Panel (b) Calibration plot of non-background-subtracted peak current versus glucose concentration (R²=0.9992).

FIG. 69 shows Panel (a) Non-background-subtracted of LSVs for a PANI-GNPs-GOx-PPMP/GME, in the oxygen saturated PBS/0.1 mM Mn(CH₃COO)₂ solution in the range of 0.005 to 14.0 mM of glucose contain 1 mM Dopamin vs Ag/AgCl (Scan rate of 0.05 Vs⁻¹) and panel (b) Calibration plot of non-background-subtracted peak current versus glucose concentration (R²=0). 9984).

FIG. 70 shows panel (a) Non-background-subtracted of LSVs for a PANI-GNPs-GOx-PPMP/GME, in the oxygen saturated PBS/0.1 mM Mn(CH₃COO)₂ solution in the range of 0.005 to 16.0 mM of glucose contain 1 mM Fructose vs Ag/AgCl (Scan rate of 0.05 Vs⁻¹) and panel (b) Calibration plot of non-background-subtracted peak current versus glucose concentration (R²=0.9993).

FIG. 71 shows panel (a) Non-background-subtracted of LSVs for a PANI-GNPs-GOx-PPMP/GME, in the oxygen saturated PBS/0.1 mM Mn(CH₃COO)₂ solution in the range of 0.005 to 15.0 mM of glucose contain 1 mM Uric acid vs Ag/AgCl (Scan rate of 0.05 Vs⁻¹) and panel (b) Calibration plot of non-background-subtracted peak current versus glucose concentration (R²=0.9993).

FIG. 72 shows panel (a) Non-background-subtracted of LSVs for a PANI-GNPs-GOx-PPMP/GME, in the oxygen saturated PBS/0.1 mM Mn(CH₃COO)₂ solution in the range of 0.005 to 16.0 mM of glucose contain 1 mM Aspartame vs Ag/AgCl (Scan rate of 0.05 Vs⁻¹). (b) Calibration plot of non-background-subtracted peak current versus glucose concentration (R²=0.9990).

FIG. 73 shows panel (a) Non-background-subtracted of LSVs for a PANI-GNPs-GOx-PPMP/GME, in the oxygen saturated PBS/0.1 mM Mn(CH₃COO)₂ solution in 0.5 mM glucose 6 Times.

FIG. 74 shows panel (A) Schematic illustration of the electrochemical modification of the PANI-GOX-PPMP/Au electrode and its SEM image, panel (B) Schematic illustration of the electrochemical modification of the PANI-GOX-PPMP/AuNPs/Au electrode and its SEM image, and panel (C) Schematic illustration of the electrochemical modification of the PANI-GOX-PPMP-AuNPs/Au electrode and its SEM image.

FIG. 75 shows panel (A) Background-subtracted of LSVs for a PANI-GOX-PPMP/Au electrode, in the oxygen saturated PBS/0.1 mM Mn(CH₃COO)₂ solution in the range of 0.05 to 15.0 mM of glucose vs Ag/AgCl (Scan rate of 0.05 Vs⁻¹) and panel (B) Calibration plot of background-subtracted peak current versus glucose concentration (R²=0.9991).

FIG. 76 shows panel (A) Background-subtracted of LSVs for a PANI-GOX-PPMP/AuNPs/Au electrode, in the oxygen saturated PBS/0.1 mM Mn(CH₃COO)₂ solution in the range of 0.01 to 15.0 mM of glucose vs Ag/AgCl (Scan rate of 0.05 Vs⁻¹) and panel (B) Calibration plot of background-subtracted peak current versus glucose concentration (R²=0.9991).

FIG. 77 shows panel (A) Background-subtracted of LSVs for a PANI-GOX-PPMP-AuNPs/Au electrode, in the oxygen saturated PBS/0.1 mM Mn(CH₃COO)₂ solution in the range of 0.005 to 16.0 mM of glucose vs Ag/AgCl (Scan rate of 0.05 Vs⁻¹) and panel (B) Calibration plot of background-subtracted peak current versus glucose concentration (R²=0.9990).

FIG. 78 shows panel (A) Background-subtracted of CVs for a PANI-GOX-PPMP-AuNPs/Au electrode, in the oxygen saturated PBS/0.1 mM Mn(CH₃COO)₂ solution in the range of 0.005 to 16.0 mM of glucose vs Ag/AgCl (Scan rate of 0.05 Vs⁻¹) and panel (B) Calibration plot of background-subtracted peak current versus glucose concentration (R²=0.9991).

FIG. 79 shows panel (A) Background-subtracted LSV plots of the PANI-GOX-PPMP-AuNPs/Au electrode, at different scan rates of 0.01, 0.03, 0.05, 0.07, 0.10, 0.20 Vs⁻¹ vs Ag/AgCl in 0.050 mM glucose in PBS pH 7/0.1 mM Mn(CH₃COO)₂ in an oxygen saturated solution and panel (B) Linear relationship between the oxidation peak current and the square root of the scan rate (R²=0.9995).

FIG. 80 shows panel (A) Background-subtracted LSVs plots of PANI-GOX-PPMP-AuNPs/Au electrode, in the oxygen saturated PBS/0.1 mM Mn(CH₃COO)₂ solution containing Dopamine in the range of 0.005 to 16.0 mM of glucose vs Ag/AgCl (Scan rate of 0.05 Vs⁻¹) and panel (B) Calibration plot of background-subtracted peak current versus glucose concentration (R²=0.9985).

FIG. 81 shows panel (A) Storage stability of current of the PANI-GOX-PPMP-AuNPs/Au biosensor in PBS (pH 7), at 300 μM concentration of glucose and panel (B) Storage stability of potential of the PANI-GOX-PPMP-AuNPs/Au biosensor in PBS (pH 7), at 300 μM concentration of glucose.

FIG. 82 shows panel (A) SEM images of PANI-GOX-PPMP/Au electrode, panel (B) PANI-GOX-PPMP/AuNPs/Au electrode, and panel (C) PANI-GOX-PPMP-AuNPs/Au electrode

FIG. 83 shows Cyclic voltammetric sweep of 20 cycles with potential ranging from −0.25 to 0.45 V/Ag/AgCl, (Scan rate of 0.05 Vs⁻¹) for the electropolymerization of the aniline composite.

FIG. 84 shows panel (a) Background-subtracted Linear sweep voltammograms for a PANI-GOX-PPMP-AuNPs/Au electrode, in the oxygen saturated PBS/0.1 mM Mn(CH₃COO)₂ solution containing 0.1 mM Ascorbic acid in the range of 0.005 to 16.0 mM (R²=0.9993) of glucose vs Ag/AgCl (Scan rate of 0.05 Vs⁻¹) with lower detection limit of 0.001 mM and panel (b) Calibration plot of background-subtracted peak current versus glucose concentration.

FIG. 85 shows panel (a) Background-subtracted Linear sweep voltammograms for a PANI-GOX-PPMP-AuNPs/Au electrode, in the oxygen saturated PBS/0.1 mM Mn(CH₃COO)₂ solution containing 0.1 mM Aspartame in the range of 0.01 to 16.0 mM (R²=0.9991) of glucose vs Ag/AgCl (Scan rate of 0.05 Vs⁻¹) with lower detection limit of 0.0026 mM and panel (b) Calibration plot of background-subtracted peak current versus glucose concentration.

FIG. 86 shows panel (a) Background-subtracted Linear sweep voltammograms for a PANI-GOX-PPMP-AuNPs/Au electrode, in the oxygen saturated PBS/0.1 mM Mn(CH₃COO)₂ solution containing 0.1 mM caffeine in the range of 0.01 to 15.0 mM (R²=0.9991) of glucose vs Ag/AgCl (Scan rate of 0.05 Vs⁻¹) with lower detection limit of 0.003 mM and panel (b) Calibration plot of background-subtracted peak current versus glucose concentration.

FIG. 87 shows background-subtracted Linear sweep voltammograms for a PANI-GOX-PPMP-AuNPs/Au electrode, in the oxygen saturated PBS/0.1 mM Mn(CH₃COO)₂ solution in 300 μM glucose concentration vs Ag/AgCl (Scan rate of 0.05 Vs⁻¹) during 35 days of storage.

FIG. 88 shows panel (a) Background-subtracted linear sweep voltammograms for a Nafion/PPMP-GOx-BSA/Au electrode, in the oxygen saturated (pH 7.0) (PBS)/0.1 mM Mn(CH₃COO)₂ solution in the range of 0.10-12.0 mM of Glucose vs Ag/AgCl (scan rate of 0.05 Vs⁻¹). Panel (b) Calibration plot of background-subtracted peak current versus glucose concentration (circles). Solid line is best-fit.

FIG. 89 shows amperometric responses of the PPMP-GOx-BSA/Au electrode, in the oxygen saturated (pH 7.0) (PBS)/0.1 mM Mn(CH₃COO)₂ solution upon the addition of glucose at 0.84V, vs Ag/AgCl.

FIG. 90 shows amperometric responses of the Nafion PPMP-GOx/Au electrode and Nafion PPMP-GOx-BSA/Au electrode, in the oxygen saturated (pH 7.0) (PBS)/0.1 mM Mn(CH₃COO)₂ solution upon the addition of glucose at 0.84V, vs Ag/AgCl.

FIG. 91 shows SEM images of Nafion/PPMP-GOx-BSA/Au membrane spin coated with 20 μL solution containing PPMP, GOx, BSA, and glutaraldehyde and 7 μL of 0.05% Nafion.

FIG. 92 shows amperometric responses of the Nafion/PPMP-GOx-BSA/Au electrode, in the oxygen saturated (pH 7.0)/0.1 mM Mn(CH₃COO)₂ solution upon the addition of glucose with various working potentials (0.79, 0.81, 0.84, and 0.86 V vs Ag/AgCl).

FIG. 93 shows corresponding calibration plot shows the dependence of current response on the concentration of glucose with various working potentials (0.79, 0.81, 0.84, and 0.86 V vs Ag/AgCl). The detection limit is shown for each potential.

FIG. 94 shows a non-limiting, representative voltammogram.

FIG. 95 shows a non-limiting, representative calibration curve.

FIG. 96 shows non-limiting, exemplary data. (Panel A) Non-background-subtracted LSVs for a PANI-GNPs-GOx-PPMP/GME, in the oxygen saturated PBS/0.1 mM Mn(CH₃COO)₂ solution in the range of 0.002 to 16.0 mM of glucose vs Ag/AgCl (Scan rate of 0.05 Vs⁻¹). (Panel B) Calibration plot of non-background-subtracted peak current versus glucose concentration (R²=0.9991).

FIG. 97 shows Catalytic effect of MnP using PPMP enzyme and GOx followed by electron transfer to O₂ to form H₂O₂ which causes glucose to be oxidized to gluconolactone.

FIG. 98 shows non-limiting, exemplary data. (Panel A) Non-background-subtracted of CVs for a PANI-GNPs-GOx-PPMP/GME, in the oxygen saturated PBS/0.1 mM Mn(CH₃COO)₂ solution in the range of 0.001 to 16.0 mM of glucose vs Ag/AgCl (Scan rate of 0.05 Vs⁻¹). (Panel B) Calibration plot of non-background-subtracted peak current versus glucose concentration (R²=0.9990).

FIG. 99 shows non-limiting, exemplary experimental data. (Panel A) Chronoamperograms at the PANI-GNPs-GOx-PPMP/GME in the oxygen saturated PBS/0.1 mM Mn(CH₃COO)₂ containing different concentrations of glucose from 0.001-16.0 mM at 0.73V potential versus Ag/AgCl (Scan rate of 0.05 Vs⁻¹). (Panel B) Calibration curve for glucose peak current versus glucose concentration (R²=0.9991).

FIG. 100 shows non-limiting, exemplary experimental data. (Panel A) Chronoamperograms at the PANI-GNPs-GOx-PPMP/GME in the oxygen saturated PBS/0.1 mM Mn(CH₃COO)₂ containing different concentrations of glucose from 0.005-16.0 mM in 1.0 mM ascorbic acid at 0.73V potential versus Ag/AgCl (Scan rate of 0.05 Vs⁻¹). (Panel B) Calibration curve for glucose peak current versus glucose concentration (R²=0.9995).

FIG. 101 shows non-limiting, exemplary experimental data. (Panel A) Non-background-subtracted LSV plots of the PANI-GNPs-GOx-PPMP/GME, at different scan rates of 0.01, 0.03, 0.05, 0.07, 0.10, 0.20 Vs⁻¹ vs Ag/AgCl in 1.0 mM glucose in PBS pH 7/0.1 mM Mn(CH₃COO)₂ in an oxygen saturated solution: (Panel B) Linear relationship between the oxidation peak current and the square root of the scan rate (R²=0.9994).

FIG. 102 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in the range of 0.005 to 16.0 mM of glucose containing 1 mM ascorbic acid vs Ag/AgCl (Scan rate of 0.05 Vs⁻¹). (Panel B) Calibration plot of non-background-subtracted peak current versus glucose concentration (R²=0.9993).

FIG. 103 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in the range of 0.005 to 16.0 mM of glucose containing 1 mM Caffeine vs Ag/AgCl (Scan rate of 0.05 Vs⁻¹). (Panel B) Calibration plot of non-background-subtracted peak current versus glucose concentration (R²=0.9992).

FIG. 104 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in the range of 0.005 to 14.0 mM of glucose containing 1 mM Dopamin vs Ag/AgCl (Scan rate of 0.05 Vs⁻¹). (Panel B) Calibration plot of non-background-subtracted peak current versus glucose concentration (R²=0.9984).

FIG. 105 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in the range of 0.005 to 16.0 mM of glucose containing 1 mM Fructose vs Ag/AgCl (Scan rate of 0.05 Vs⁻¹). (Panel B) Calibration plot of non-background-subtracted peak current versus glucose concentration (R²=0.9993).

FIG. 106 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in the range of 0.005 to 15.0 mM of glucose containing 1 mM Uric acid vs Ag/AgCl (Scan rate of 0.05 Vs⁻¹). (Panel B) Calibration plot of non-background-subtracted peak current versus glucose concentration (R²=0.9993).

FIG. 107 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in the range of 0.005 to 16.0 mM of glucose containing 1 mM Aspartame vs Ag/AgCl (Scan rate of 0.05 Vs⁻¹). (Panel B) Calibration plot of non-background-subtracted peak current versus glucose concentration (R²=0.9990).

FIG. 108 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in the range of 0.01 to 16.0 mM of glucose containing 10% diet coke vs Ag/AgCl (Scan rate of 0.05 Vs⁻¹). (Panel B) Calibration plot of non-background-subtracted peak current versus glucose concentration (R²=0.9996).

FIG. 109 shows non-limiting, exemplary experimental data. (Panel A) Non-background-subtracted LSVs for a PANI-GNPs-GOx-PPMP/GME, in the oxygen saturated PBS/0.1 mM Mn(CH₃COO)₂ solution in 0.5 mM glucose 6 Times.

FIG. 110 shows non-limiting, exemplary illustrations of the Phase I and Phase II designs, encompassing exemplary features described herein.

FIG. 111 shows a non-limiting, exemplary schematic of the catalytic effect of PPMP enzyme.

FIG. 112 shows non-limiting, exemplary experimental data. (Panel A) Background-subtracted of SWVs for a CPs-GOx-GNPs-PPMP/SPE, (Panel B) Calibration plot of background-subtracted peak current versus glucose concentration (R²=0.9997).

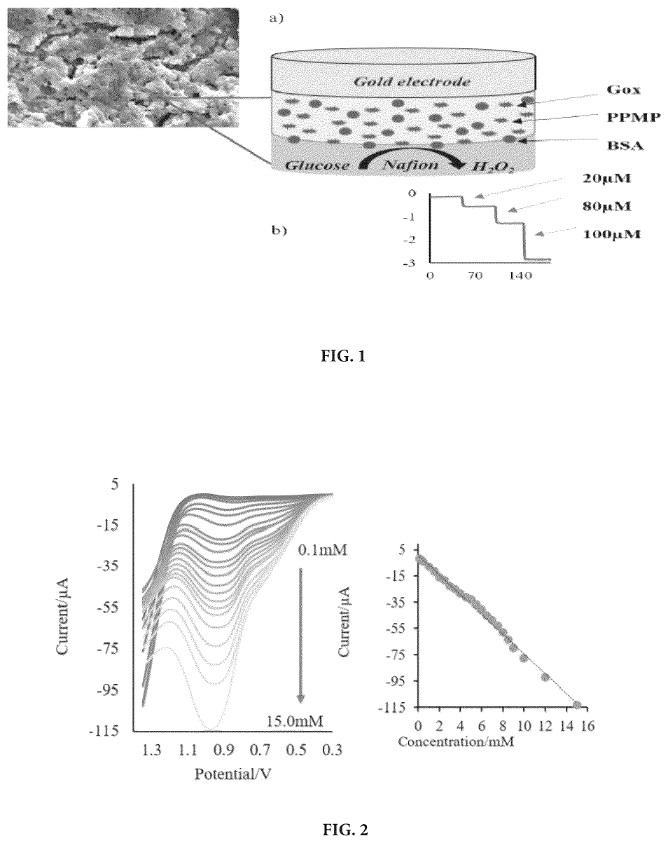

### DETAILED DESCRIPTION OF THE INVENTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the United States patent law definition of “comprising” and is therefore interpreted to be an open term meaning “at least the following.” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Electrochemical Sensor

Aspects of the invention are drawn towards an electrochemical sensor and a sensor system for electrochemically detecting and/or measuring at least one analyte in a fluid, as well as a method for manufacturing the electrochemical sensor, which at least partially overcome the shortcomings of known devices and methods.

The term “analyte” can refer to a compound being present in a fluid sample, wherein the presence and/or the concentration of the analyte can be of interest to the subject or to a medical staff, such as to a medical doctor. In embodiments, the analyte can comprise at least one compound which can participate in the metabolism of the user or the patient, such as at least one metabolite. As an example, the at least one analyte can be selected from the group consisting of glucose and hydrogen peroxide. In embodiments, other types of analytes can be used and/or any combination of analytes can be determined. Without restricting further applications, this disclosure is described with reference to the detection, measurement, and/or monitoring of glucose in a fluid sample.

A “biosensor” can refer to a sensor which utilizes biological components for a response to an analyte. In embodiments, the biological component can comprise enzymes, antibodies, or DNA. In embodiments, the biosensor can selectively respond to an analyte. In embodiments, the biosensor can generate an electric signal.

An “electrochemical sensor” can refer to a sensor that can perform at least one electrochemical measurement, such as a plurality or series of electrochemical measurements, to detect the at least one substance as comprised within the body fluid by using an electrochemical method. In embodiments, the terms “electrochemical” and “electroanalytical” can be used interchangeably. For example, “electrochemical measurement” can refer to the detection of an electrochemically detectable property of a substance, such as an electrochemical detection reaction, by employing electroanalytic methods. In embodiments, electroanalytical methods can comprise potentiometry, coulometry, voltammetry, and amperometry. Thus, for example, the electrochemical detection reaction can be detected by applying and comparing one or more electrode potentials. The electrochemical sensor can be adapted to generate at least one electrical measurement signal which can directly or indirectly indicate a presence and/or an extent of the electrochemical detection reaction, such as at least one current signal and/or at least one voltage signal. The measurement can be a qualitative and/or a quantitative measurement.

The electrochemical sensor can be an amperometric sensor and/or a voltammetric sensor. An electrochemical sensor is one that functions by the production of a current when a potential is applied between at least two electrodes. The current generated can be proportional to the concentration of chemical species in solution. An amperometric sensor is a sensor that measures the current at a constant potential, and a voltammetric sensor is a sensor that measures current as a function of applied potential. As used herein, the term “amperometry” can refer to measuring the electric current between a pair of electrodes that are driving an electrolysis reaction. For example, the current is proportional to the concentration of an analyte. As used herein, the term “voltammetry” can refer to techniques in which the relation between current and voltage is observed during an electrochemical process. As used herein, the term “voltammogram” can refer to a graph of current vs. potential. In embodiments, voltammetry can comprise cyclic voltammetry and linear sweep voltammetry. In embodiments, the voltammetry can comprise square wave voltammetry. In embodiments, the voltammetry can comprise ion transfer voltammetry or ion exchange voltammetry. In embodiments, “cyclic voltammetry” can refer to a potentiodynamic electrochemical measurement wherein a working electrode potential is cycled, and the resulting current is measured. In embodiments, “linear voltammetry” can refer to a voltammetric method wherein the current at the working electrode is measured while the potential between the working electrode and reference electrode is swept linearly in time. In embodiments, “square wave voltammetry” can refer to a differential technique that uses a combined square wave and staircase potential applied to a working electrode. In embodiments, the electrochemical sensor can be an amperometric sensor or a voltammetric sensor.

Embodiments of the electrochemical sensor can comprise one or more electrodes. An “electrode” can refer to a part of the electrochemical sensor which can contact the fluid sample, directly or via at least one semipermeable membrane or layer.

In embodiments, the sensor can comprise a counter electrode, a working electrode, a reference electrode, or a combination thereof. In embodiments, the at least one electrode can be embodied in a manner that oxidative processes and/or reductive processes can take place at selected surfaces of the electrode.

The at least one electrode of the electrochemical sensor can comprise a working electrode and a counter electrode, wherein the working electrode further includes one or more enzymes, wherein one or more enzymes are configured for providing a reaction with an analyte while the counter electrode is maintained free from the enzyme.

A “counter electrode” can refer to an electrode that ensures that the correct potential difference between the reference electrode and the working electrode is being applied. For the example, the counter electrode can be any inert material, such as copper, carbon, ruthenium, rhodium, palladium, silver, rhenium, osmium, iridium, platinum, gold, graphite, and combinations and alloys thereof. In embodiments, the counter electrode is a platinum electrode or a carbon electrode.

A “working electrode” can refer to an electrode on which a reduction or oxidation reaction occurs. For example, the working electrode can be gold or carbon. For example, the working electrode can be paper or paper-based. For example, the working electrode can be a 3 mm gold working electrode.

In embodiments, the electrochemical sensor can further comprise a reference electrode. For example, electrical potential can be applied between the working electrode and the reference electrode, and raw current generated thereby can be measured between the working electrode and the counter electrode.

A “reference electrode” can refer to an electrode that has a stable and well-known electrode potential. For example, the reference electrode can be silver (Ag), silver/silver chloride (Ag/AgCl) electrode or saturated calomel electrode (SCE). In other non-limiting embodiments, the reference electrode can be a standard hydrogen electrode (SHE), a reversible hydrogen electrode (RHE), copper-copper (II) sulfate electrode, or a palladium-hydrogen electrode. For example, the reference electrode can be silver (Ag).

In embodiments, the electrodes described herein can comprise screen-printed electrodes (SPEs). As used herein, the term “screen printed electrode” can refer to an electrochemical device that is manufactured by printing inks on substrates. In embodiments the inks can comprise carbon, silver, gold, platinum, or a combination thereof. In embodiments, the substrates can comprise plastic, paper, metal, ceramic substrates, or a combination thereof. For example, the SPE comprises a paper-based SPE. For example, the paper-based SPE can be coated in gold. For example, the paper-based SPE can be coated in gold nanoparticles. In embodiments, the SPE can comprise a Conductive polymer(CP)-GNP-GOX-PPMP/SPE. For example, the SPE can comprise a PANI-GNP-GOX-PPMP/SPE.

In embodiments, the electrodes described herein can comprise a paper-based electrode. For example, the paper-based electrode can comprise paper-supported electrodes and paper-like electrodes. For example, the paper-based electrode can be constructed from materials and processes know in the art, see. e.g., Yao et al., Advanced Science, 2017, 4, 1700107. For example, the paper-based electrode can comprise a gold and/or carbon-based paper. For example, the paper-based electrode can comprise a cellulose-based paper. In embodiments, the paper-based electrode can be coated in a polymer, a metal, or combination thereof. For example, the paper-based electrode can be coated in gold, PANI, or a combination thereof. In embodiments, the paper-based electrode can comprise graphene-cellulose paper.

In embodiments, the sensors described herein can comprise a paper-based sensor. For example, the paper-based sensor can comprise a gold and/or carbon-based paper.

In embodiments, the one or more electrodes can be surrounded by or partially surrounded by a coating. The coating can provide a biocompatible interface between the sensor and the fluid sample that wicks the sample over the electrodes. In embodiments, the entirety of the electrode can be surrounded by the coating. In embodiments, about 10%, about 20%, about 30%, about 40%, about 50%, about 60%, about 70%, about 80%, about 90%, or about 100% of the electrode can be surrounded by the coating. In embodiments, only about the portion of the electrode that is in contact with the fluid sample can be surrounded by the coating.

In embodiments, the coating can be deposited on the surface of one or more electrodes of the electrochemical sensor. For example, the coating can be deposited onto the gold electrodes by spin coating, immersion, electrodeposition, or electropolymerization.

In an embodiment, for example, a gold working electrode is polished with an alumina paste slurry on a polishing pad prior to the coating being deposited onto the gold electrode by spin coating. After spin coating, the coating was dried at room temperature for a period of time, such as 1 hour. In embodiments, a conductive polymer, such as Nafion™, can be deposited onto the coating by spin coating.

In another embodiment, for example, a gold working electrode can be immersed in a coating comprising a polymer, such as aniline, and then the coating can be immobilized on the electrode, such as by electropolymerization. For example, a coating comprising manganese peroxidase, glucose oxidase, aniline, and BSA can be immobilized on the working electrode by electropolymerization. Variability of membrane thickness can be achieved by adjusting the applied potential during electropolymerization.

In another embodiment, for example, the Screen-Printed Electrodes (SPEs) can be subjected to an electrodeposition procedure. In embodiments, a composite material can be deposited on the working electrode. In embodiments, the composite material comprises a conductive polymer. In embodiments, the composite comprises of PPMP, GOX (glucose oxidase), GNPs (gold nanoparticles), and CPs (conductive polymers). In embodiments, Electropolymerization can be carried out on the SPEs by sweeping the potential. In further embodiments, the SPE is cleaned via cyclic voltammetry prior to deposition.

In embodiments, two or more enzymes can be distributed within the coating. For example, the two or more enzymes can comprise glucose oxidase and a peroxidase.

A “glucose oxidase” can refer to a polypeptide which catalyzes the oxidation of glucose to gluconic acid with the concomitant production of hydrogen peroxide (H₂O₂). In embodiments, glucose oxidase can be recombinantly produced. For example, glucose oxidase from Aspergillus niger can be recombinantly produced for use in embodiments of the invention.

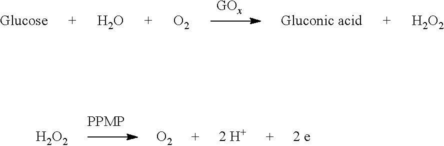

Oxidation of H₂O₂ produces electrons which are measured by the electrode. The current produced by the oxidation of H₂O₂ is proportional to the concentration of glucose in the blood. A “peroxidase” can refer to an enzyme that catalyzes the oxidation of a molecule along with peroxide. This includes all enzymes that contain peroxidase activity, including, but not limited to manganese peroxidase, horseradish peroxidase, myeloperoxidase, cytochrome C peroxidase, glutathione peroxidase, microperoxidase, lactoperoxidase, or soybean peroxidase. In embodiments, the peroxidase can be manganese peroxidase.

For example, the catalytic process to detect glucose can comprise a glucose oxidase (GOx) reaction which generates hydrogen peroxide (H₂O₂). This produced hydrogen peroxide, in the presence of glucose, serves as a substrate for manganese peroxidase (MnP). MnP utilizes hydrogen peroxide and a reducing agent (for example a low-molecular-weight compound) to oxidize Mn (II) to Mn (III) at its heme active site. The presence of Mn (III) at the enzyme's active site enables it to effectively catalyze the oxidation of glucose to gluconolactone. The overall reaction can be summarized as follows:

Glucose+O₂→Gluconolactone+H₂O₂ (by GOx)

Mn(II)+H₂O₂→Mn(III)+H₂O(by MnP)+1/2O₂

The catalytic cycle of these enzymes assists in the detection and quantification of glucose by engaging in redox reactions that involve glucose, molecular oxygen, hydrogen peroxide, and manganese ions.

In embodiments, the manganese peroxidase can be a fungal manganese peroxidase encoded by a gene or gene fragment isolated from Agaricus bisporus, Coprinopsis cinerea, Galerina marginate, Laccaria bicolor, Pleurotus ostreatus, Schizophyllum Commune, Auricularia delicata, Serpula lacrymans, Coniophora puteana, Botryobasidion botryosum, Punctularia strigosozonata, Dacryopinax sp., Gloeophyllum trabeum, Fomitiporia mediterranea, Jaapia argillacea, Dichomitus squalens, Ceriporiopsis subvermispora, Fomitopsis pinicola, Phanerochaete carnosa, Phanerochaete chrysosporium, Postia placenta, Trametes versicolor, Wolfiporia cocos, Heterobasidion annosum, Stereum hirsutum, Piriformospora indica, Cryptococcus neoformans, Tremella mesenterica, Wallemia sebi, Puccinia graminis, Melampsora laricis-populina, Ustilago maydis, Malassezia globose. In embodiments, the manganese peroxidase is encoded by a gene or gene fragment isolated from Phanerochaete chrysosporium.

In embodiments, the enzyme, such as the peroxidase and/or the glucose oxidase, can be recombinantly produced. The term “recombinant protein” or “recombinantly-produced protein” can refer to a peptide or protein produced using non-native cells that do not have an endogenous copy of DNA able to express the protein. The cells produce the protein because they have been genetically altered by the introduction of the appropriate nucleic acid sequence. The recombinant protein will not be found in association with proteins and other subcellular components normally associated with the cells producing the protein.

For example, the enzyme can be produced in a plant or plant cell. For example, manganese peroxidase as described herein can be recombinantly produced in corn. Non-limiting examples of plant or plant cells can comprise corn, soy bean, or rice.

The coating can further comprise one or more components, such as bovine serum albumin (BSA), glutaraldehyde, a conductive polymer, an anti-fouling polymer, a nanoparticle, or any combination thereof.

Bovine serum albumin (BSA) as a spherical protein and can be used in biochemical studies. For example, BSA in combination with the detection enzymes demonstrated excellent conductivity, biocompatibility, and multifunctionality, and was environmentally friendly and highly stable. As described herein, the detection limit of the electrochemical sensor was improved when using BSA, which, without wishing to be bound by theory, can be due to strong intermolecular interactions among BSA molecules and the enzymes.

In certain embodiments, aldehydes and other low-molecular weight crosslinkers known in the art can be added to the coating. In certain embodiments, glutaraldehyde, for example, can be added to the coating, as it reacts rapidly and is more efficient than other aldehydes in generating stability. Further, glutaraldehyde can enhance the specificity and effectiveness of enzyme immobilization and enzyme cross-linking.

For example, the coating can comprise about 0.1 mg to about 20 mg PPMP, about 0.1 mg to about 15 mg Gox, about 0.5 μL to about 50 μL of 45 μM BSA, and about 5 μL to about 50 μL glutaraldehyde (2.5%) in about 0.01 M to about IM PBS at about pH 7. In certain embodiments the coating can comprise about 10 mg of PPMP, about 7 mg of GOx, about 40 μL of 45 μM BSA, and about 25 μL glutaraldehyde (2.5%) in 0.5 mL of 0.1 M PBS (pH 7).

In an embodiment, the coating comprises manganese peroxidase, glucose oxidase, and bovine serum albumin (BSA).

In an embodiment, the coating comprises manganese peroxidase, glucose oxidase, bovine serum albumin (BSA), and glutaraldehyde.

“Conductive polymer” can refer to a polymer which can conduct electricity. The electrical conductivity can be tuned depending on the type of polymer(s) used. Non-limiting examples of such conductive polymers comprise a perfluorosulphonic acid polymer, 3,4-ethylene dioxythiophene (EDOT), polyaniline (PANI), pyrrole, polypyrrole, and/or Poly(3,4-ethylenedioxythiophene)-poly(styrenesulfonate) (PEDOT:PSS). For example, the conductive polymer can be Sulfonated Tetrafluoroethylene (Nafion™). For example, the conductive polymer can comprise polypyrrole and polyaniline.

For example, Nafion™ is a perfluorosulphonic acid polymer that can be used as a semipermeable membrane for biosensors due to excellent conductivity of the membrane. Nafion™ shows adhesion, catalytic activity, and biocompatibility and improves the biosensor stability. Moreover, membranes coated with Nafion™ show excellent glucose and oxygen diffusivity.

In embodiments, the conductive polymer can be deposited on the coating that surrounds the one or more electrodes. For example, the conductive polymer can be deposited onto the coating that surrounds the one or more electrodes by spin coating, immersion, or electropolymerization. As used herein, the coating surrounding one or more electrodes can be referred to as a “membrane”. As used herein, the terms “coating” and “membrane” can be used interchangeably.

In other embodiments, the conductive polymer can be admixed within the coating prior to the coating being deposited on the one or more electrodes.

For example, a mixture comprising manganese peroxidase, a polymer such as aniline, glucose oxidase, and BSA can be immobilized onto an electrode by electropolymerization. For example, polymerizations can be conducted by the cyclic voltammetric sweep of 10 to 25 cycles with potential ranging from −0.25 to 0.45 V/Ag/AgCl, at a scan rate of 0.05 Vs⁻¹. In embodiments, 20 cycles can be used.

Electrode fouling can involve the passivation of an electrode surface by an anti-fouling agent that forms an increasingly impermeable layer on the electrode, thereby inhibiting direct contact of an analyte of interest with the electrode surface for electron transfer. Embodiments as described herein can comprise compositions and methods to prevent electrode fouling, for example incorporating an anti-fouling polymer into the coating. Non-limiting examples of anti-fouling polymers comprise poly(ethylene glycol) (PEG), zwitterionic polymers, poly(hydroxyfunctional acrylates), poly(2-oxazoline)s, poly(vinylpyrrolidone), poly(glycerol), peptides and peptoids.

The “nanoparticle” can refer to a particle having a size (for example, diameter of a spherical particle) from about 1 nm to about 1000 nm. Such as from about 1 nm to 1000 nm. Such as about 1 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, or about 1000 nm. Non-limiting examples of the nanoparticle material includes graphene, borophene, gold, carbon nanotubes, or platinum. For example, the nanoparticle material can comprise any material to amplify the electrode surface area.

In embodiments, the electrochemical sensor can comprise electrodes comprising polyaniline (PANI), gold nanoparticles (GNPs), glucose oxidase (GOx), and plant-produced manganese peroxidase (PPMP). In embodiments, the electrochemical sensor can comprise polyaniline (PANI), gold nanoparticles (GNPs), glucose oxidase (GOx), and plant-produced manganese peroxidase (PPMP) screen printed electrode.

In embodiments, the nanoparticle can be deposited on the one or more electrodes prior to at least partially surrounding the electrode with the coating, thereby forming a nanoparticle layer between the one or more electrode and the coating.

In other embodiments, the nanoparticle can be admixed within the coating prior to the coating being deposited on the one or more electrodes.

In embodiments, the coating has a thickness of about 100 μm to about 1 mm. For example, the thickness of the coating can be less than about 1 mm thick. For example, the thickness of the coating can be about 100 μm, about 200 μm, about 300 μm, about 400 μm, about 500 μm, about 600 μm, about 700 μm, about 800 μm, about 900 μm, or about 1 mm. For example, the thickness of the coating can be controlled by the electropolymerization cycle. That is, variability of membrane thickness can be achieved by adjusting the applied potential during electropolymerization.

In embodiments, the one or more electrodes can be present in a microfluidic device in communication with a microfluid channel. A “microfluidic device” can refer to a device comprising fluidic structures and internal channels having microfluidic dimensions. These fluidic structures can include chambers, valves, vents, vias, pumps, inlets, nipples, and detection means, for example. Microfluidic channels can comprise fluid passages having variable length and at least one internal cross-sectional dimension that is less than approximately 500 μm to 1000 μm, such as between approximately 0.1 μm and approximately 500 μm.

In embodiments, electrochemical sensor can comprise the one or more electrodes immobilized on a solid support member. Non-limiting examples of the composition of the solid support member comprise plastic, cardboard, glass, plexiglass, tin, paper, or a combination thereof.

In embodiments, the support member comprises a screen-printed member. For example, embodiments comprise a screen-printed electrode (SPE) based sensor, which has advantages such as robustness, excellent detection limitations, selectivity, sensitivity portable, cost-effective with potential of mass production and this electrode in paper based.

In embodiments, the electrochemical sensor can be miniaturized, thereby improving the portability, ease of handling, ease of incorporation into arrays, and the like. The term “miniaturization” can refer to a manufacturing technique to reduce the size of the electrochemical sensor.

In embodiments, the integration of compositions, processes, and methods described herein can provide increased sensitivity and selectivity, increased stability, cost-effectiveness and accessibility, miniaturization, longevity and reduced drift, improved signal detection, and closed-loop integration. For example, the integration of screen-printed electrodes, nanoparticles, Recombinant Mn Peroxidase from Corn (PPMP), glucose oxidase, conductive polymers, and the utilization of Square Wave Voltammetry (SWV) can provide increased sensitivity and selectivity, increased stability, cost-effectiveness and accessibility, miniaturization, longevity and reduced drift, improved signal detection, and closed-loop integration. For example, increased sensitivity and selectivity can refer to the ability to detect glucose accurately and selectively. For example, stability can refer to consistent performance over time and various conditions. For example, cost-effectiveness and accessibility can refer to creating an affordable product that is accessible to a wider population. For example, miniaturization can increase convenience for wearability. For example, longevity and reduced drift can extend the senor's lifespan and minimize measurement errors. For example, improved signal detection can enhance the detection and interpretation of glucose levels. For example, closed-loop integration can enable real-time adjustments and automated glucose management.

Aspects of the invention are further drawn to a “sensor system.” A sensor system can refer to a device which is configured for conducting at least one medical analysis. For example, the sensor system can be a device configured for performing at least one diagnostic purpose and/or a monitoring purpose, such as a system comprising at least one electrode as described herein for performing the at least one medical analysis. The sensor system can, for example, comprise an assembly of two or more components that can interact with each other, for example to perform one or more diagnostic and/or monitoring purposes, such as to perform the medical analysis. Specifically, the two or more components can perform at least one detection of the at least one analyte in the body fluid and/or in order to contribute to the at least one detection of the at least one analyte in the body fluid.

Method of Detection

Aspects of the invention are drawn towards methods of electrochemical detection of glucose in a fluid sample.

Further aspects of the invention are drawn towards methods of measuring or determining glucose concentration in a fluid sample.

For example, embodiments comprise exposing a fluid sample obtained from a subject to an electrochemical sensor as described herein and detecting the current generated from the oxidation of H₂O₂ during said exposing, wherein current corresponds to the concentration of glucose in the fluid sample.

In embodiments, the coating at least partially surrounding the electrodes allows selective partitioning of glucose directly from a fluid sample.

A “fluid sample” can refer to any liquid sample containing or suspected of containing the analyte(s) of interest. In embodiments, the fluid sample can be a body fluid. In other embodiments, the fluid sample can be a consumable.

In embodiments, the fluid sample can be a body fluid. The term “body fluid” can refer to a fluid present in a body or a body tissue of a subject and/or which can be produced by the body of the subject. For example, the body fluid can be selected from the group consisting of blood and interstitial fluid. However, additionally, or alternatively, one or more other types of body fluids can be used, such as saliva, tear fluid, urine, ascites, cerebrospinal fluid (CSF), sputum, bone marrow, synovial fluid, aqueous humor, amniotic fluid, cerumen, breast milk, bronchioalveolar lavage fluid, semen, prostatic fluid, cowper's fluid or pre-ejaculatory fluid, female ejaculate, sweat, fecal matter, hair, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood, or other body fluids.

During the detection of the at least one analyte, the body fluid can be present within the body or body tissue. Thus, the sensor system can at least be configured for detecting the at least one analyte within the body tissue.

The methods described herein can involve obtaining a fluid sample from a subject. As used herein, the phrase “obtaining a fluid sample” can refer to any process for directly or indirectly acquiring a fluid sample from a subject. For example, a fluid sample can be obtained (e.g., at a point-of-care facility, e.g., a physician's office, a hospital, laboratory facility) by procuring a tissue or fluid sample (e.g., blood draw, marrow sample, spinal tap) from a subject. Alternatively, a fluid sample can be obtained by receiving the fluid sample (e.g., at a laboratory facility) from one or more persons who procured the sample directly from the subject.

In embodiments, the method can be repeated periodically. “Periodically” can refer to performing the method at recurring or repeating intervals, such as about every one minute, about every 5 minutes, about every 30 minutes, about every one hour, about every 4 hours, about every 8 hours, about every 12 hours, about every 24 hours, about once a day, about once a week, about once a month, about every three months, about every 6 months, or about every year.

Aspects of the invention can determine a subject's blood glucose concentration. The term “blood glucose concentration” can refer to the glucose concentration in the subject's bloodstream. The normal blood glucose concentration (normoglycemia) is approximately 85-95 mg/dl in an overnight fasting state. This value varies up to 30 mg/dl if not diabetic. “Hyperglycemia” is a situation in which the blood glucose concentration is too high. Hyperglycemia can occur when blood glucose levels rise and exceed 180 mg/dl.

The term “urinary glucose concentration” can refer to the glucose concentrating in the subject's urine. The normal amount of glucose in urine is 0 to 0.8 mmol/L. A higher measurement can be a sign of health problems, such as diabetes.

In embodiments, the subject's glucose concentration can be measured once, or can be measured continuously or periodically over a period of time. The term “period of time” can refer to the period of time necessary to achieve an effect or result. For example, the period of time can comprise about 1 hour, about 2 hours, about 3 hours, about 4 hours, about 5 hours, about 6 hours, about 12 hours, about 18 hours, about 24 hours, about 36 hours, about 48 hours, or longer than about 48 hours. In embodiments, the period of time comprises about 12 or about 24 hours.

The term “continuously” can refer to without interruption or with minimal interruption. For example, continuous measurement can refer to the fact that the measurements are repeated continuously over very small intervals of time. In embodiments, the subject's glucose level can be measure continuously over a period of time.

The term “continuous glucose sensing” or “continuous glucose monitoring” can refer to the period in which monitoring of the glucose concentration of a subject's bodily fluid (e.g., blood, serum, plasma, extracellular fluid, etc.) is continuously or continually performed, for example, at time intervals ranging from fractions of a second up to, for example, 1, 2, or 5 minutes, or longer. In one exemplary embodiment, the glucose concentration of a host's extracellular fluid is measured every 1, 2, 5, 10, 20, 30, 40, 50 or 60-seconds.

In embodiments, the electrochemical sensor can detect between about 20.0 μM and about 15.0 mM glucose in a fluid sample. In embodiments, the fluid sample can comprise a PBS solution. In embodiments, the electrochemical sensor can detect about 1.0 μM, 2.0 μM, 5.0 μM, about 10 μM, about 20 μM, about 30) μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 950 μM, about 1 mM, about 2.5 mM, about 5 mM, about 10) mM, about 15 mM, and about 20 mM of glucose in a fluid sample. For example, the electrochemical sensor can detect between about 2.5 mM to about 7 mM of glucose in a fluid sample.

In embodiments, the sensor has a lower detection limit of about 2.9 μM. In embodiments the sensor has a lower detection limit of about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, about 2.5 μM, about 2.6 μM, about 2.7 μM, about 2.8 μM, about 2.9 μM, about 3.0 μM, about 10 μM, about 25 μM, about 50 μM, about 75 μM, about 100 μM, about 250 μM, about 500 μM, about 1 mM, about 2 mM, or about 5 mM.

Method of Diagnosing

Aspects of the invention are further drawn towards methods of diagnosing a subject with or at risk of a metabolic disease or disorder, such as diabetes.

The term “diagnosing” can refer to determining presence or absence of a disease, classifying a disease or a symptom, determining a severity of the disease, monitoring disease progression, forecasting an outcome of a disease and/or prospects of recovery.

The term “subject” can refer to any organism to which aspects of the invention can be performed, e.g., for experimental, diagnostic, prophylactic, and/or therapeutic purposes. Example of subjects can include be mammals, such as primates, for example humans. For veterinary applications, a wide variety of subjects will be suitable, e.g., livestock such as cattle, sheep, goats, cows, swine, and the like: poultry such as chickens, ducks, geese, turkeys, and the like; and domesticated animals such as pets, for example dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted herein or another organism that is alive. The term “living subject” can refer to the entire subject or organism and not just a part excised (e.g., a liver or other organ) from the living subject.

In embodiments, the disease can be a metabolic disease. The term “metabolic disease” or “metabolic disorder” can refer to any disease or disorder that disrupts normal metabolism, including any disease that disrupts or dysregulates biochemical reactions that function to convert food into energy, process or transport amino acids, proteins, carbohydrates (e.g., sugars, starches), or lipids (e.g., fatty acids), and the like. In embodiments, a metabolic disease results in the abnormal processing or regulation of sugars, lipids, cholesterol, and/or the metabolism of drugs (e.g., by the liver). Non-limiting examples of metabolic diseases include obesity, insulin resistance, type 2 diabetes, hyperlipidemia, non-alcoholic fatty liver disease (NAFLD), and non-alcoholic steatohepatitis (NASH), as well as the sequelae of such diseases.

For example, the term “diabetes” can refer to high blood sugar or ketoacidosis, as well as chronic metabolic abnormalities arising from a prolonged high blood sugar status or a decrease in glucose tolerance. “Diabetes” encompasses both the type I and type II (Non-Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women. For example, diabetes can refer to type 2 diabetes mellitus.

For example, embodiments can comprise exposing a fluid sample obtained from a subject to an electrochemical sensor as described herein, and detecting the current generated from the oxidation of H₂O₂ during said exposing, wherein current corresponds to the concentration of glucose in the fluid sample, and diagnosing the subject as having or at risk of having diabetes if the fasting glucose level is about 100 mg/dL to above 126 mg/dL. For example, a subject can be classified has having pre-diabetes if fasting glucose levels comprise about 100 mg/dL to about 125 mg/dL.

Aspects of the invention are also drawn towards methods of monitoring a subject with or at risk of diabetes. The term “monitoring” can refer to the act of measuring, quantifying, qualifying, estimating, sensing, calculating, interpolating, extrapolating, inferring, deducing, or any combination of these actions. For example, “monitoring” can refer to a way of getting information via one or more sensing elements, such as an electrochemical sensor as described herein. For example, “glucose level monitoring” includes monitoring blood glucose levels.

For example, embodiments can comprise exposing a fluid sample obtained from a subject to an electrochemical sensor as described herein, detecting the current generated from the oxidation of H₂O₂ during said exposing, wherein current corresponds to the concentration of glucose in the fluid sample, thereby monitoring the subject as having or at risk of having diabetes if glucose levels are elevated. For example, a subject can be at risk of having diabetes if the fasting glucose level is about 100 mg/dL to above 126 mg/dL. For example, a subject can be classified has having pre-diabetes if fasting glucose levels comprise about 100 mg/dL to about 125 mg/dL.

Method of Preventing or Treating

Aspects of the invention are further drawn towards a method of preventing or treating a subject afflicted with or at risk of diabetes.

The term “prevent.” “prevention.” or “preventing” can refer to any method to partially or completely prevent or delay the onset of one or more symptoms or features of a disease, disorder, and/or condition, such as diabetes. Prevention can be administered to a subject who does not exhibit signs of a disease, disorder, and/or condition.

The term “treating” can refer to partially or completely alleviating, ameliorating, improving, relieving, delaying onset of, inhibiting progression of, reducing severity of, and/or reducing incidence of one or more symptoms, features, or clinical manifestations of a disease, disorder, and/or condition, such as diabetes. For example, “treating” diabetes can refer to reducing or normalizing glucose levels. For example, “treating” diabetes can include lifestyle interventions (such as, diet and physical activity) to manage body weight, pharmacological interventions for weight loss, pharmacological treatment with insulin sensitizers, bariatric surgery, or a combination thereof. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

In embodiments, treatment can comprise administering to the subject one or more therapeutic agents. The term “therapeutic agent” can refer to any chemical moiety that is a biologically, physiologically, or pharmacologically active substance that acts locally or systemically in a subject. The term also can refer to any substance intended for use in the diagnosis, cure, mitigation, treatment or prevention of disease or in the enhancement of desirable physical or mental development and/or conditions in an animal or human. For example, embodiments can comprise exposing a fluid sample obtained from a subject to an electrochemical sensor as described herein, detecting the current generated from the oxidation of H₂O₂ during said exposing, wherein current corresponds to the concentration of glucose in the fluid sample, and treating the subject for diabetes if glucose levels are elevated. For example, a subject can be at risk of having diabetes if the fasting glucose level is about 100 mg/dL to above 126 mg/dL. For example, a subject can be classified has having pre-diabetes if fasting glucose levels comprise about 100 mg/dL to about 125 mg/dL

Kit

Aspects of the invention are also drawn towards a kit comprising the electrochemical sensor as described herein. The term “kit” can refer to a product (i.e., a kit of parts) comprising one package or one or more separate packages and including informational material. In embodiments, the kit can further comprise components and/or reagents that can measure blood glucose levels and/or other analytes in a subject.

In embodiments, the kit can comprise one or more disposable articles for measuring glucose concentration. The term “disposable article” can refer to a single or limited use article that is made from relatively inexpensive materials that make the article cost effective to fabricate. For example, the disposable article can be a swab, spoon, dipstick, filter paper, or test-strip.

In embodiments, the kit can comprise a medical device. The term “medical device” can refer any instrument, apparatus, implant, in vitro reagent or similar or corresponds article that is used to diagnose, prevent, or treat a disease or other condition, and does not achieve its purpose through pharmacological action within or on the body. For example, the medical device can be a sensor, such as an electrochemical sensor as described herein.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—a Bienzymatic Amperometric Glucose Biosensor Based on Using a Recombinant Mn Peroxidase from Corn and Glucose Oxidase with a Nation Membrane (Izadyar et al., Journal of Electroanalytical Chemistry 895 (2021) 115387)

Abstract

Using a recombinant enzyme derived from corn and a simple modification, we fabricated a facile, fast, and cost-beneficial biosensor to measure glucose. The Nafion/Plant Produced Mn Peroxidase (PPMP)—glucose oxidase (GOx)—Bovine serum albumin (BSA)/Au electrode showed an excellent amperometric response to detect glucose. Without wishing to be bound by theory, this biosensor can respond to a wide range of glucose, 20.0 μM-15.0 mM, and has a lower detection limit (LOD) of 2.9 μM. The reproducibility response using six electrodes is also very substantial and indicates high capability of this biosensor to detect glucose concentrations. Selectivity of this electrode was investigated in an optimized experimental solution containing 10% diet green tea with citrus containing ascorbic acid (AA), and citric acid (CA) in a wide concentration of glucose at 0.02 to 14.0 mM with an LOD of 3.1 μM. We also applied linear sweep voltammetry (LSV) and this technique shows a wide range of glucose detection. The performance and strength of this enzyme biosensor were the simplicity, wide linear ranges, sensitivities, selectivity, and low limits of detection. Without wishing to be bound by theory, the modified biosensor can monitor various biofluids.

### INTRODUCTION

In our daily lives, measuring the amount of sugar in food and drink or for healthcare is crucial.[1-3] By monitoring blood glucose for diabetic people on a regular basis, a healthy life can be ensured. Diabetes is one of the most common chronic diseases, and is due to an imbalance in the body's glucose levels. Diabetes mellitus (DM) is one of the leading causes of death in any country, and malnutrition makes diabetes one of the most common chronic diseases. Diabetes caused 1.5 million deaths in 2012. Excessive blood glucose increases the risk of cardiovascular and other diseases by another 2.2 million. As of 2019, an estimated 463 million people worldwide will suffer from diabetes, and without wishing to be bound by theory, this number can reach 700 million by 2045. [4-6] Diabetics suffer from many disorders in their body and thus their treatment is more painful as they address multiple conditions. Regardless of the available invasive techniques, non-invasive blood glucose monitoring has attracted much attention in recent years.[7,8] Among a number of glucose measurement methods, optical and electrochemical analyses have been investigated. Optical methods use color change in an index that reflects the concentration of glucose. Colors change when an enzyme reaction converts glucose to its metabolites.[9,10] Although color change provides an intuitive way to test for blood glucose, it is not effective in measuring low glucose levels. Even if small measurements can be made, it often requires pricey and not easily portable equipment,[10] therefore rendering it unsuitable for commercial use and more suitable in specialized institutional settings such as hospitals.[12] Electrochemical techniques[13] involve a simple, sensitive and selective method of operation and are widely used in glucose sensors.[14] Amperometry detection based on enzymatic reactions has played an important role in personal glucose level monitoring. Research has been done to improve the structure of the electrode surface to create more sensitive measurements and a selective mode of performance. Research teams around the world have devised different methods for measuring glucose. Biosensors are known to be the most suitable device for painless and accurate measurement of blood glucose levels. Enzymatic biosensors are considered one of the most selective, sensitive and reliable tools in electrochemical determinations[14] In essence, GOx oxidizes glucose through a sub-reaction to produce gluconic acid and hydrogen peroxide (H₂O₂).

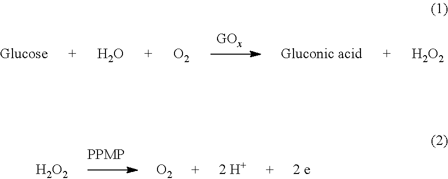

For direct detection of analytes, a positive potential higher than 1 V versus Ag/AgCl is used for direct detection of analytes, but the high potential also facilitates oxidation of ascorbic acid, acetaminophen, uric acid, and lactic acid, which reduces the selectivity of the biosensor. Complementary mediators such as ferrocene or ferricyanide derivatives are also used with GOx, to improve performance and minimize the oxidation of the interfering species. However, it can be noted that these biosensors cannot be used in vivo due to the toxicity of these materials. Some GOx biosensors use enzymes whose structure has been modified to facilitate direct electron exchange between electrodes and embedded enzymes such as flavin adenine dinucleotide (FAD: a subunit of the enzyme). Although this is progress in providing electrocatalytic intermediates for direct electron transfer, some problems, such as the existence of a suitable enzyme in terms of activity, price, and by-products, are still challenging issues for glucose measurements. Horseradish peroxidase (HRP) with GOx is an enzyme pair used to detect glucose.[17] However, HRP exhibits several disadvantages including high cost and limited reaction conditions. In this work we introduce a new enzyme, recombinant Mn Peroxidase from corn (plant-produced manganese peroxidase, PPMP) for glucose detection to solve these problems. PPMP is an alternative enzyme, a fungal manganese peroxidase (MnP), which is produced in the cost-effective plant-based production system.[17] The gene for the enzyme (Genbank accession #: L29039) was cloned from the white rot fungus, Phanerochaete chrysosporium and expressed recombinantly in corn grain. We obtained results showing that this PPMP is an improved enzyme over horseradish peroxidase (HRP) for creation of a selective and sensitive electrochemical biosensor.[19]

Electrochemical biosensors using enzymes require adequate selectivity against interfering materials, reproducibility, fast response time, sensitivity to low levels of analyte and selectivity for the specific analyte. Bovine serum albumin as a spherical protein, is widely used in biochemical studies.[20,21] The BSA in combination with the detection enzymes demonstrated excellent conductivity, biocompatibility, and multifunctionality, and was environmentally friendly and highly stable.[22] In this work we found an improvement in the detection limit when using BSA, which was probably due to strong intermolecular interactions among BSA molecules and the enzymes. We also applied Nafion™, which is a perfluorosulphonic acid polymer and widely used as a semipermeable membrane to fabricate biosensors due to excellent conductivity of the membranes.[23] Nafion™ also shows adhesion, catalytic activity, and biocompatibility and improves the biosensor stability[23] Moreover, membranes coated with Nafion™ show excellent glucose and oxygen diffusivity.[25] Glutaraldehyde also is added to our membrane composition. It reacts rapidly and is more efficient than other aldehydes in generating stability. Glutaraldehyde is commercially available with a low cost in addition to its high reactivity[26]. The long-term stability of biosensors can be guaranteed using glutaraldehyde, and is one of the important components in the design of biosensors. It has been shown to enhance the specificity and effectiveness of enzyme immobilization and enzyme cross-linking.[14]

In this article we report a new amperometric biosensor for glucose detection. The results show that this recombinant Mn peroxidase can be a new enzyme for measuring glucose due to its high sensitivity for the development of electrochemical biosensors.

### 2. Experimental

### 2.1. Materials and Reagents

The glucose solution was prepared using 0.1 M phosphate buffer (NaPB; pH7.0) using NaH₂PO₄ (monobasic) Na₂HPO₄ (dibasic), which were purchased from Fisher Scientific. Manganese (II) acetate tetrahydrate 9.99% (Mn(CH₃COO)₂), bovine serum albumin (BSA), 25% solution of glutaraldehyde, Glucose Oxidase from Aspergillus niger Type II, ≥10,000 units/g solid (160 kDa), Nafion™ perfluorinated resin solution, and α-D-glucose anhydrous, 96%, were purchased from Sigma-Aldrich (St. Louis, MO). All of the solutions were prepared in Milli-Q deionized water (18.2 MΩ) unless otherwise noted. All of the experiments were conducted at laboratory room temperature (20° C.).

### 2.2 Electrochemical Measurements

All voltammetry and amperometric measurements were performed using a computer-controlled CHI660D electrochemical workstation (CH Instruments, Austin, TX). Experiments were carried out in a three-electrode cell with a platinum wire (0.25 mm diameter, 99.9%, Alfa Aesar, Ward Hill, MA) as a counter electrode and a 5 mm-diameter gold electrode as the working electrode, and Ag/AgCl (3 M KCl) as the reference electrode. The oxidation of glucose on the modified gold electrode was quantified with LSV and amperometry in 0.1 M phosphate buffer (pH 7.0), and 0.1 mM (Mn CH₃COO)₂) solution. The solution had undergone saturation with oxygen for 10 min before a certain amount of glucose was added. The Amperometry technique was applied at constant potential and constant stirring when glucose was injected at constant time intervals. Scanning Electron Microscope (SEM) images were recorded on a SNE-4500 M Plus Tabletop Scanning Electron Microscope (Nano Images. LLC).

### 2.3 PPMP Enzyme Preparation

Corn containing recombinant MnP was ground to a fine flour and extracted using 50 mM sodium tartrate pH 4.5 (Fisher Scientific; BP352-500) at a ratio of 1.5 L per 1 kg of corn. The pH of the sodium tartrate was adjusted to 4.5 using tartaric acid. Mixing was conducted for 1 h in an ice bath. A filtering aid (diatomaceous earth) was added to the slurry and filtered through a 15 cm Whatman #1 filter in a Buchner funnel using a vacuum pump, and the filtered solids were discarded. The filtrate was then concentrated to half of its volume using a Pellicon-2 tangential flow filtration (TFF) ultrafiltration unit (Millipore). Solid ammonium sulfate was gradually added to 95% SAS, and the slurry was mixed for approximately 1 h. Filtration aid was then added, the slurry was filtered with a Buchner funnel, and the filtrate was discarded while the precipitate was saved. The precipitate was then added to 50 mM sodium tartrate buffer at pH 4.5 at a concentration of 1 mg of precipitate per 1 mL of buffer and mixed for 30 min. The slurry was then filtered (with filtration aid), and the resulting filtrate was saved, the precipitate was discarded. The filtrate was concentrated and desalted using the TFF (Pellicon 2, 10 kDa MW cutoff). The filtrate was passed over a Giga Cap S-650 M column to remove residual corn proteins, and the flow-through contained purified MnP. The combined fractions containing the MnP were concentrated using TFF and were lyophilized.

### 2.4 Electrode Preparation

The gold working electrode (5 mm in diameter) was polished with an alumina paste slurry (0.3, 0.1 and 0.05 μm) on the polishing pad (Buehler. Ltd., Lake Bluff. IL). The enzyme solution was prepared by dissolving 10.0 mg PPMP. 7.0 mg of GOx. 40 μL of 45 μM BSA, and 25 μL glutaraldehyde (2.5%) in 0.5 mL of 0.1 M PBS (pH 7). The enzyme layer was deposited onto the gold electrodes by spin coating. After spin coating, the enzyme film was dried at room temperature for 1 hour. Then 0.05% Nafion™ polymer was also deposited by spin coating on the enzyme membrane. The electrodes are stored at 4° C. when not in use.

### 3. Results and Discussion

### 3.1 Linear Sweep Voltammetry (LSV) of the Nafion™/PPMP-Gox-PBS/Au Electrode

We validated the electrochemical behavior of the modified biosensor using the LSV method. The biosensor was fabricated under the following optimized setup, with spin coating of 15 μL of a solution containing the enzymes as described in methods, followed by application of 7 μL of the 0.05% Nafion™ solution by spin coating after one hour when the membrane dried. As the LSV of the Nafion/PPMP-GOx-BSA/Au electrode shows in FIG. 2 panel a, an oxidation peak has appeared at ≈+0.96 V with a scan rate of 0.05 Vs⁻¹ (United States convention used to report LSVs). The sensing behavior of the Nafion/PPMP-GOx-BSA/Au electrode was studied with the successive addition of glucose at a range of 0.1-15.0 mM in a PBS (pH 7.0)/0.1 mM Mn (CH₃COO)₂ solution saturated with oxygen gas before glucose was added. Oxidation peaks proportionally increase with the addition of glucose. The increase in glucose concentration was associated with a sharp increase in current and a change in the peak oxidation potential to a higher value, indicating an increase in the local concentration of oxygen on the surface of the Nafion/PPMP-GOx-BSA/Au electrode. The R² values of the regression lines that fitted to the calibration curves were 0.9938 (FIG. 2 panel b). The limit of detection (LOD) of 19.5 μM was calculated for S/N=3 and S=(3×std dev)+blank.[27]

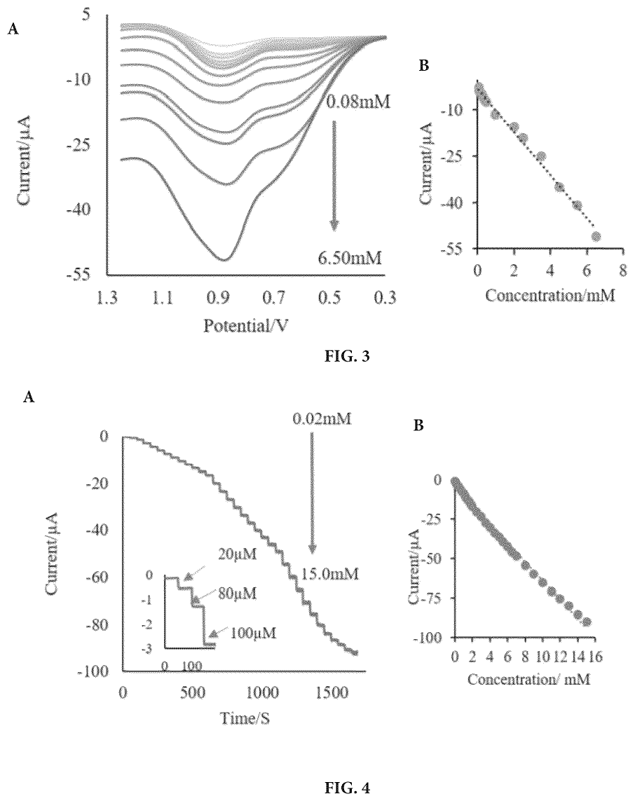

The modified electrode shows a unique property, a high degree of stability associated with an electrically active surface using this enzyme and the composition associated with this enzyme. Different compositions were investigated using the PPMP. We applied 20 μL of a solution containing 10.0 mg PPMP. 7.0 mg of GOx, 40 μL of 45 μM BSA, and 25 μL glutaraldehyde (2.5%) in 0.5 mL of 0.1 M PBS and then after 1 hour, 7 μL of 0.05% Nafion solution was spin coated onto the enzyme membrane to fabricate the Nafion/PPMP-GOx-BSA/Au electrode. Background-subtracted LSVs show a wide linear range of 0.10-12.0 mM, and an LOD of 0.025 mM was calculated for S/N=3. The R² values of the regression lines that fitted to the calibration curves were 0.9948. All measurements were made in a PBS (pH 7.0)/0.1 mM Mn (CH₃COO)₂ solution, which was saturated with oxygen gas before glucose was added. The peak potential when the highest concentration of enzyme is used is 1.10 V, which in comparison with the optimum conditions of 0.96 V is shifted to a more positive potential, which indicates that the oxidation of H₂O₂ is more difficult for a thicker membrane. Another membrane with PPMP-GOx-BSA/Au composition was tested similarly to the optimum conditions, only this film was not coated with Nafion. FIG. 3 panel a shows the background-subtracted LSVs of the PPMP-GOx-BSA/Au electrode, which exhibits an oxidation peak at ≈+0.87 V at a scan rate of 0.05 Vs⁻¹. This shows the sensing behavior of the PPMP-GOx-BSA/Au electrode with addition of glucose at the range of 0.08-6.5 mM in an optimized experimental solution. The R² values of the regression lines that fitted to the calibration curves were 0.9923 with an LOD of 2.9 μM, that was calculated for S/N=3 (FIG. 3 panel b). The LSV results indicated that membrane with Nafion™ shows a better sensitivity and reproducibility with a wider range of glucose concentration measurements compared to the membrane without Nafion™.

### 3.2 Amperometric Responses of the Nafion™ PPMP-Gox-BSA Au Electrode to Detect Glucose

Amperometry, because of its hydrodynamic conditions, shows higher sensitivity and selectivity compared to LSV. Therefore, we evaluated the performance of this biosensor, applying the amperometric I-t responses to demonstrate the efficiency of the Nafion/PPMP-GOx-BSA/Au electrode. The steady-state amperometric results of the modified electrode were investigated under optimized experimental conditions after successive additions of glucose from 0.02 to 15.0 mM (FIG. 4 panel a). We applied this technique at constant potential and constant stirring when glucose was injected at constant time intervals. As is shown, rapid and sensitive responses to glucose were achieved and we observed the maximum current response at 0.84 V. We had 30 injections of glucose from 1.0 μL of 0.2 M to 20.0 μL of 1.0 M in 10.0 mL of optimized experimental solution. The R² values of the regression lines that fitted to the calibration curves were 0.9943 and an LOD of 2.9 μM was calculated for S/N=3 (FIG. 4 panel b). However, when the concentration of glucose exceeded 15.0 mM, the current no longer changed, showing a response proportional to the glucose concentration because oxygen accumulated on the surface of the electrode. Amperometry of the PPMP-GOx-BSA/Au biosensor also was investigated. The results show a less stable membrane with lower sensitivity compared to the membrane spin coated with Nafion. Also, the results of amperometric measurement of the Nafion/PPMP-GOx/Au electrode without using BSA and glutaraldehyde were compared with the optimized electrode using BSA, which shows greater sensitivity.

Results of our work show the superiority of the PPMP enzyme in the early stages of application. Table 1 shows the detection limit for the Nafion™/PPMP-GOx-BSA/Au electrode compared with other glucose electrochemical sensors. The results show that the ability to measure glucose by this sensor with a single-layer membrane and without adding any nanomaterials, mediators, or conductive polymers is fully competitive with more expensive and sophisticated sensors. It can be noted that the Nafion™/PPMP-GOx-BSA/Au biosensor fabrication is very fast and inexpensive and can measure up to 15 mM glucose. The results correlate to the cost difference of PPMP and HRP also show a significant difference in the price of these two enzymes (Table 2) [32].

TABLE 1
comparison of glucose determination with differently modified electrodes.

| | | | | LOD |
|---|---|---|---|---|
| Ref. | Method | Enzyme | Sensor Property | (μM) |
| [17] | Cyclic voltammetry | HRP and GOx | co-assembled onto carbon nanotubes (CNTs) | 7.0 |
| [28] | Amperometric | GOx | GOx in 0.1% Nafion-ethanol | 45.0 |
| | | | immobilized on pristine multiwalled | |
| | | | carbon nanotubes (PMWCNT) | |
| [29] | Amperometric | GOx | polypyrrole (Ppy) on the surface of a | 200.0 |
| | | | GOx/gold nanoparticles/graphite rod | |
| | | | electrode | |
| [30] | Amperometric | GOx | chitosan-glucose oxidase immobilized | 5.0 |
| | | | on polypyrrole/Nafion/functionalized | |
| | | | multi-walled carbon nanotubes in a | |
| | | | bio-nanohybrid film | |
| [31] | Amperometric | HRP and GOx | bienzyme glucose biosensor based on | 400.0 |
| | | | horseradish peroxidase and glucose | |
| | | | oxidase cross-linked to multiwall | |
| | | | carbon nanotubes | |
| This work | Amperometric | PPMP and GOx | Nafion/Recombinant Mn Peroxidase | 2.9 |
| | | | from Corn/Gox Au electrode | |

TABLE 2
Cost comparison of PPMP and HRP

| | MnP from corn | | |
|---|---|---|---|
| Characteristic | (PPMP) | HRP | Reference |
| MW | 53,000 Da | 43,000 Da | |
| # isozymes | 1 | 7 | |
| Enzyme # | 1.11.1.13 | 1.11.1.7 | |
| Substrate | H₂O₂, Mn | H₂O₂ | [32] |
| pH optimum | 5-6 | 7 | |
| Concentration in | 5 g/kg corn kernels | 156 mg/kg roots | |
| biomass | | | |
| Purified cost | $50-$75/g* | $1250/g* | [32] |
| Source | Infinite | BBI Solutions, | |
| | Enzymes, LLC | Sigma Chemical | |
| | | Co. | |

*GMP at scale

### 3.3 Effect of Potential

We validated the effect of different applied potentials on the amperometric response under optimized experimental conditions with the successive addition of glucose under stirred conditions. The amperometric responses of the Nafion/PPMP-GOx-BSA/Au biosensor were evaluated at different applied potentials of 0.79, 0.81, 0.84, and 0.86 V. At 0.84 V, by adding 1.0 μL of 0.2 mM glucose at the first step, the current changed immediately and after 50s it reached equilibrium when the glucose concentration in the solution became uniform by stirring. However, in higher and lower potentials than 0.84V, changing of current takes longer and 60 to 70s are needed for the current to reach equilibrium and as the concentration increases it takes longer for the current achieve stability. In addition, at 0.84 V, we achieved an LOD of 2.9 μM. Therefore, without wishing to be bound by theory, this potential can be the optimum potential as we achieved a lower detection limit and greater reproducibility.

### 3.4 Morphology Studies

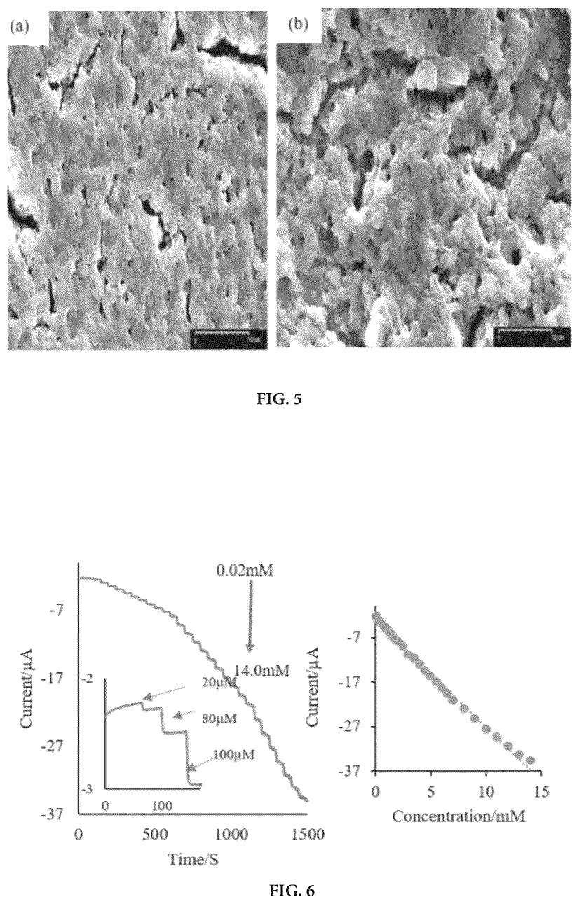

Scanning Electron Microscope (SEM) images were recorded at different magnification for PPMP-GOx-BSA/Au. and Nafion™/PPMP-GOx-BSA/Au electrodes. FIG. 5 panel b shows the surface morphology of a Nafion™/PPMP-GOx-BSA/Au membrane is rough with cluster structures and a perforated surface and a larger surface area compared to a PPMP-GOx-BSA/Au membrane without Nafion™ (FIG. 5 panel a). The formation of a compact, highly ordered protein film when that film is coated with Nafion™ can explain this result. The amperometry results in better immobilization of the enzyme on the matrix coated with Nafion. However, when we applied 20 μL of composition solution, which is 5 μL higher than the optimum condition, the SEM image demonstrated larger clusters in the morphology of the membrane. Nevertheless, the enzyme activity is lower than the optimized conditions due to the increase in membrane thickness.

### 3.5 Selectivity, and Reproducibility of the Nafion/PPMP-Gox-BSA/Au Sensor

The interference of electroactive compounds present in physiological samples of glucose such as ascorbic acid (AA), and citric acid (CA), cause problems in accurate determination of glucose. Since human blood always contains many compounds like AA that are oxidized easily, the influence cannot be ignored. [28, 29] To validate the selectivity of the Nafion™/PPMP-GOx-BSA/Au biosensor, we validated the amperometry response of the electrode in optimized experimental conditions in a solution containing 10% diet green tea with citrus. This sample contains green tea. CA, sodium hexametaphosphate, water, natural flavor. AA, phosphoric acid, potassium sorbate, aspartame, acesulfame potassium, calcium disodium. EDTA, and caramel color. As FIG. 6 panel a shows, notwithstanding the presence of interfering materials in this sample, the response of the sensor is a sign of its high selectivity. The amperometric response of the biosensor shows a rapid, sensitive and selective response to glucose in a wide concentration of 0.02 to 14.0 mM. We applied a potential of 0.84 V with 29 injections of glucose from 1.0 μL of 0.2 M to 10.0 μL of 1.0 M in 10.0 mL of optimized experimental solution containing 10% diet green tea with citrus. The R² values of the regression lines that fitted to the calibration curves were 0.9972, despite AA, CA, and other contaminants being present in this sample (FIG. 6 panel b). The fabricated biosensor can selectively detect glucose with an LOD of 3.1 μM calculated for S/N=3.

The reproducibility of the fabricated biosensor was also studied using six successively modified sensors. The amperometric response under optimized experimental conditions after successive additions of glucose from 0.02 to 15.0 mM concentration shows superb reproducibility of this biosensor with the lower detection limit of 3.1 μM and at higher concentration range of 13.2 mM. We also validated the reproducibility of the five additional modified sensors in the same optimized experimental solution containing 10% diet green tea with citrus. Our results show an impressive detection limit and wide concentration range with a lower detection limit of 3.3 μM and the highest concentration of range of 13.0 mM. Thus, the modified electrode can be used as a highly reproducible, sensitive and selective glucose sensor with ideal resistance against interfering species, such as ascorbic acid and citric acid. Also noteworthy is that the sensors were simply and rapidly fabricated.

Conclusions

In this work, we validated an amperometric enzyme-based Nafion™/PPMP-GOx-BSA/Au biosensor, which is not only cost effective, simple, sensitive, selective, and stable but also able to respond to a wide range of glucose concentrations. The Nafion™/PPMP-GOx-BSA/Au biosensor was successfully used in LSV, and amperometric measurement of glucose to detect various concentrations of this important compound. The amperometric response indicates the high capability of this biosensor to detect a wide range of glucose concentrations (3.1 μM to 13.2 mM). Furthermore, the optimized experimental solution of 10% diet green tea with citrus, containing ascorbic acid and citric acid, which can interfere in physiological samples, was used to study the selectivity of this electrode. Our studies in the samples and conditions show that this biosensor has the ability to respond with excellent selectivity toward glucose monitoring in the wide range of 3.3 μM to 13.0 mM. The studies we have done so far on this new recombinant enzyme from the corn kernel production system indicate that this enzyme with low cost, reproducibility, sensitivity, and selectivity can be widely used as an excellent alternative enzyme to detect glucose and can easily compete with conventional enzymes such as HRP for these measurements. In addition, without wishing to be bound by theory, in the next stage of our work, by using nanomaterials and conductive polymers, we can significantly improve the LOD of the PPMP based biosensor. This biosensor will be much more effective in development and construction of biosensors than current designs in real sample use such as measuring various biofluids.

### REFERENCES CITED IN THIS EXAMPLE

[1] S. Emaminejad, W. Gao, E. Wu, Z. A. Davies, H. Y. Y. Nyein, S. Challa, S. P. Ryan, H. M. Fahad, K. Chen, Z. Shahpar, S. Talebi, C. Milla, A. Javey, Autonomous sweat extraction and analysis applied to cystic fibrosis and glucose monitoring using a fully integrated wearable platform. PNAS, 114 (2017) 4625-4630.
[2] A. J. Andodkar, J. Wang, Non-invasive wearable electrochemical sensors: a review Trends Biotechnol. 32 (2014) 363-371.
[3] H. Lee, Y. J. Hong, S. Baik, T. Hyeon, D. H. Kim, Enzyme-Based Glucose Sensor: From Invasive to Wearable Device, Adv. Healthcare Mater. 7 (2018) 1701150.
[4] Centers for Disease Control and Prevention, National Diabetes Statistics Report, (2020).
[5] N. H. Cho, J. E. Shaw, S. Karuranga, Y. Huang, J. D, da Rocha Fernandes, A. W. Ohlrogge, IDF Diabetes Atlas: global estimates of diabetes prevalence for 2017 and projections for 2045. Diabetes Res Clin Pract. 138 (2018) 271-281
[6] J. M. O'Connell, S. M. Manson, Diabetes Care, 42 (2019) 1609-1611.
[7] A. Kandwal, T. Igbe, J. Li, Y. Liu, S. Li, L. W. Y. Liu, Z. Nie, Highly Sensitive Closed Loop Enclosed Split Ring Biosensor with High Field Confinement for Aqueous and Blood-Glucose Measurements, Sci. Rep. 10 (2020) 4081.
[8] L. Lipani, B. G. R. Dupont, F. Doungmene, F. Marken, R. M. Tyrrell, H. Richard, R. H. Guy, A. Ilie, Noninvasive, transdermal, path-selective and specific glucose monitoring via a graphene-based platform, Nat. Nanotechnol. 13 (2018) 504-511.
[9] J. S. Kim, H. B. Oh, A. H. Kim, J. S. Kim, E. S. Lee, J. Y Baek, K. S. Lee, S. C. Chung, J. H. Jae-Hoon Jun, A study on detection of glucose concentration using changes in color coordinates, Bioengineered. 8 (2017) 99-104.
[10] Y. Lin, M. Zhao, Y. Guo, X. Ma, F. Luo, L. Guo, B. Qiu, G. Chen, Z. Lin, Multicolor Colormetric Biosensor for the Determination of Glucose based on the Etching of Gold Nanorods, Sci. Rep, 6 (2016) 37879.
[11] E. Y. Park, J. Baik, H. Kim, S. M. Park, C. Kim, Ultrasound-modulated optical glucose sensing using a 1645 nm laser, Sci. Rep, 10 (2020) 13361.
[12] B. H. Hou, H. Takanaga, G. Grossmann, L. Q. Chen, X. Q. Qu, A. M. Jones, S. Lalonde, O. Schweissgut, W. Wiechert, W. B. Frommer, Optical sensors for monitoring dynamic changes of intracellular metabolite levels in mammalian cells, Nat. Protoc. 6 (2011) 1818.
[13] S. Amemiya, J. Kim, A. Izadyar, B. Kabagambe, M. Shen, R. Ishimatsu, Electrochemical Sensing and Imaging Based on Ion Transfer at Liquid/Liquid Interfaces. Electrochim. Acta, 110 (2013) 836-845.
[14] H. Teymourian, A. Barfidokht, J. Wang, Electrochemical glucose sensors in diabetes management: an updated review (2010-2020), Chem. Soc. Rev., 49 (2020) 7671-7709.
[15] J. D. Newman, S. J. Setford, Enzymatic Biosensors, Mol. Biotechnol. 32(2006) 249-268.
[16] F. Palmisano, P. G. Zambonin, D. Centonze, M. Quinto, A Disposable, Reagentless, Third-Generation Glucose Biosensor Based on Overoxidized Poly(pyrrole)/Tetrathiafulvalene Tetracyanoquinodimethane Composite, Anal. Chem, 74 (2002) 5913.
[17] H. Yang, C. Gong, L. Miao, F. A. Xu, Glucose Biosensor based on Horseradish Peroxidase and Glucose Oxidase Co-entrapped in Carbon Nanotubes Modified Electrode, Int. J. Electrochem. Sci, 12 (2017) 4958-4969.
[18] J. Järvinen, S. Taskila, R. Isomäki, H. Ojamo, Screening of white-rot fungi manganese peroxidases: a comparison between the specific activities of the enzyme from different native producers, AMB Express, 2 (2012) 62.
[19] A. Izadyar, U. Tran, E E, Hood, Recombinant Mn Peroxidase from Corn Grain Has an Excellent Electrocatalytic Effect in a Designed Amperometric Biosensor to Detect Hydrogen Peroxide at Low Concentrations, ACS Sustainable Chem. Eng. 7 (2019) 19434-19441.
[20] A. Akhavan, H. R. Kalhor, M. Z Kassaee, N. Sheikh, Hassanlou, Radiation synthesis and characterization of protein stabilized gold nanoparticles, M. Chem. Eng. J. 159 (2010) 230-235.
[21] Z. Dai, A. Yang, X. Bao, R. Yang, Facile Non-Enzymatic Electrochemical Sensing for Glucose Based on Cu₂O-BSA Nanoparticles Modified GCE, Sensors. 19 (2019), p. 2824.
[22] C. Y. Hu, D. P. Yang, F. J. Zhu, F. J. Jiang, S. Y. Shen, J. L. Zhang, Enzyme-Labeled Pt@BSA Nanocomposite as a Facile Electrochemical Biosensing Interface for Sensitive Glucose Determination, ACS Appl. Mater. Interfaces, 6 (2014) 417Q-4178.
[23] A. Balwani, E. M. Davis, Anomalous, Multistage Liquid Water Diffusion and Ionomer Swelling Kinetics in Nafion and Nafion Nanocomposites, ACS Appl. Polym. Mater. 2 (2020) 40-54.
[24] E. Flampouri, S. Kintzios, Nafion and Polylysine Treated PEDOT Mammalian Cell Biosensor, Procedia Eng. 25 (2011) 976-979.
[25] J. C. Chou, S. H. Lin, T. Y. Lai, P. Y. Kuo, C. H. Lai, Y. H. Nien, T. Y. A. Su, Facile Fabrication of a Potentiometric Arrayed Glucose Biosensor Based on Nafion-GOx/GO/AZO, Sensors. 20(2020) 964.
[26] I. Migneault, C. Dartiguenave, M. J. Bertrand, K. C. Waldron, Glutaraldehyde: behavior in aqueous solution, reaction with proteins, and 1!1)plication to enzyme crosslinking, Bio Techniques 37(2004)790-802.
[27] D. C. Harris, Quantitative Chemical Analysis, 10th ed.; W. H. Freeman and Company: New York, (2020) 83-91.
[28] K. S. B. Dinesb, S. Devi, U. M. Krishnan, Achieving a Stable High Surface Excess of Glucose Oxidase on Pristine Multiwalled Carbon Nanotubes for Glucose Quantification, ACS Appl. Bio Mater., 2 (2019) 1740-1750.
[29] N. German, A. Ramanavicius, A. Ramanaviciene, Amperometric Glucose Biosensor Based on Electrochemically Deposited Gold Nanoparticles Covered by Polypyrrole, Electroanalysis, 29 (2017) 1267-1277.
[30] B. K. Shrestha, R. Ahmad, H. M. Mousa, LG. Kim, J. I. Kim, High-performance glucose biosensor based on chitosan-glucose oxidase immobilized polypyrrole/Nafion/functionalized multi-walled carbon nanotubes bio-nanohybrid film, J Colloid Interface Sci, 482(2016) 39-47.
[31] S. Xu, H. Qi, S. Zbou, X. Zhang, C. Zhang, Mediatorless amperometric bienzyme glucose biosensor based on horseradish peroxidase and glucose oxidase cross-linked to multiwall carbon nanotubes, Microchim Acta 181 (2014) 535-541.
[32] D. R. Walwyn, S. M. Huddy, E. P. Rybicki, Techno-Economic Analysis of Horseradish Peroxidase Production Using a Transient Expression System in Nicotiana benthamiana: Ap Biochem Biotechnol 175 (2015) 841-854.

### Example 2—Enhance the Sensitivity and Stability of the Biosensor Using Conduction Polymer EDOT

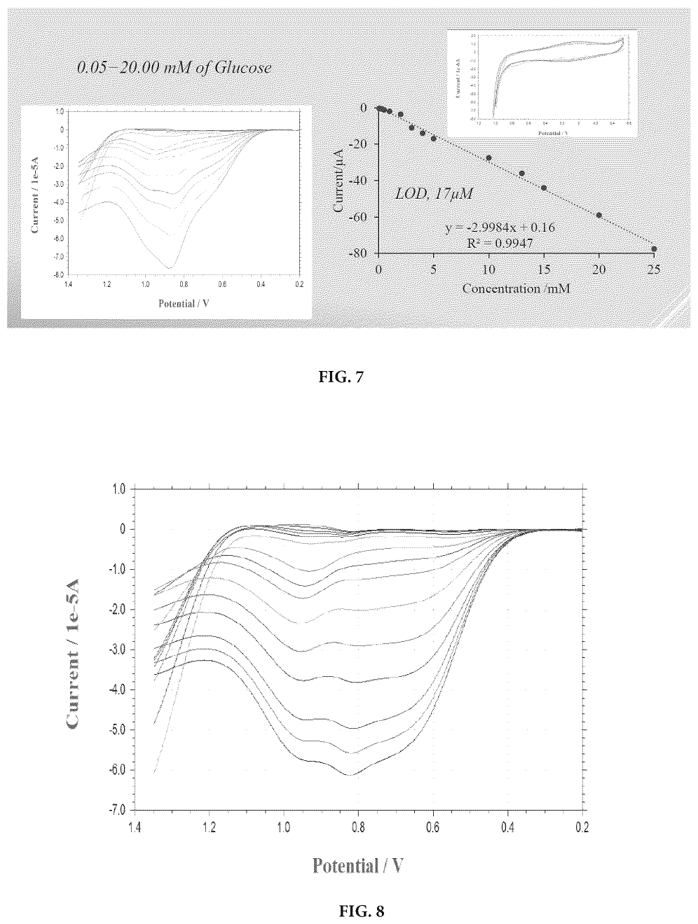

Two microliters of PPMP, BSA, Glut, GOx were added then the electrode was (FIG. 7)
Electrode was electropolymerized with EDOT for 5 cycles on polymerized Au electrode and then two 10 microliters of PPMP, BSA, Glut, GOx were added then. (FIG. 8)

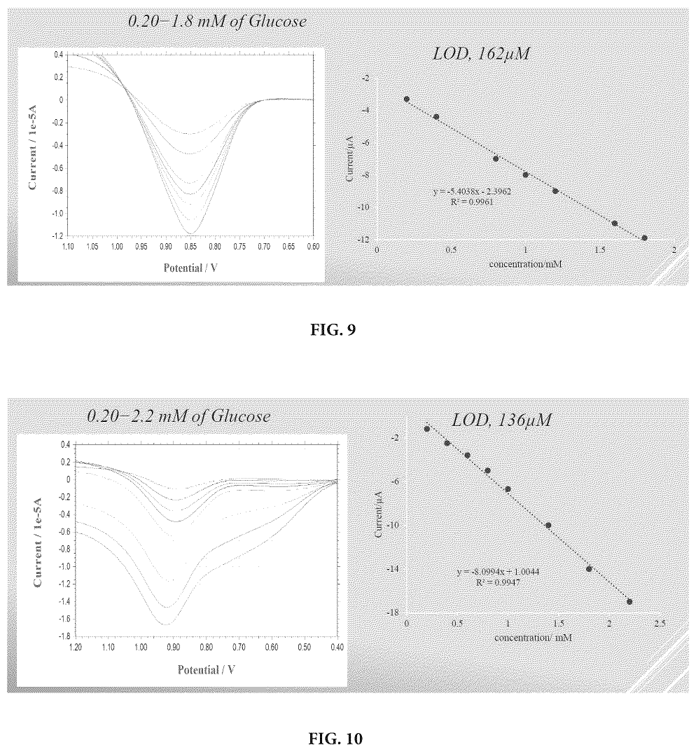

Enhance the sensitivity and stability of the biosensor using nanoparticles and conductive polymer.
In situ, (nanocomposite) electropolymerization polyaniline, GOx, PPMP, BSA, Au nanoparticle (spin coated)/Au. (FIG. 9). Nafion/PPMP-Gox-BSA/Au Electrode 0.10-15.0 mM of Glucose, oxidation peak 0.95V.
polyaniline, GOx, PPMP, BSA, Au, nanoparticle (spin coated)/Au (FIG. 10)

### Example 3—Amperometric Biosensor for Glucose Determination Based on a Recombinant Mn Peroxidase from Corn Cross-Linked to a Gold Electrode

Abstract—Using a recombinant enzyme derived from corn and a simple modification, we fabricated a facile, fast, and cost beneficial biosensor to measure glucose. The Nafion™/Plant Produced Mn Peroxidase (PPMP)—glucose oxidase (GOx)—Bovine serum albumin (BSA)/Au electrode showed an excellent amperometric response to detect glucose. This biosensor can respond to a wide range of glucose—20.0 μM-15.0 mM and has a lower detection limit (LOD) of 2.90 μM. The reproducibility response using six electrodes is also very substantial and indicates the high capability of this biosensor to detect a wide range of 3.10±0.19 μM to 13.2=1.8 mM glucose concentration. Selectivity of this electrode was validated in an optimized experimental solution contains 10% diet green tea with citrus containing ascorbic acid (AA), and citric acid (CA) in a wide concentration of glucose at 0.02 to 14.0 mM with an LOD of 3.10 μM. Reproducibility was also validated using 4 electrodes in this sample and shows notable results in the wide concentration range of 3.35±0.45 μM to of 13.0±0.81 mM. We also used other voltammetry methods to evaluate this biosensor. We applied linear sweep voltammetry (LSV) and this technique shows a wide range of 0.10-15.0 mM to detect glucose with a lower detection limit of 19.5 μM. The performance and strength of this enzyme biosensor were the simplicity, wide linear ranges, sensitivities, selectivity, and low limits of detection. Without wishing to be bound by theory, the modified biosensor can be used for monitoring various biofluids.

### Example 4—Development of a Nano-Biosensor to Detect Glucose to Detect for Diabetics, Using Recombinant Manganese Peroxidase from Corn Grain

Introduction

Aspects of the invention are at the forefront of one of the most important branches of agricultural chemistry: using enzymes to develop and modify a biosensor for diabetes. Importantly, these enzymes are manufactured in a corn kernel biofactory. Aspects of the invention are drawn towards the miniaturization of a significantly improved glucose biosensor that has increased sensitivity and longevity for diabetes sufferers. This aspect is dependent on using enzymes produced from an agricultural crop.

Project Background.

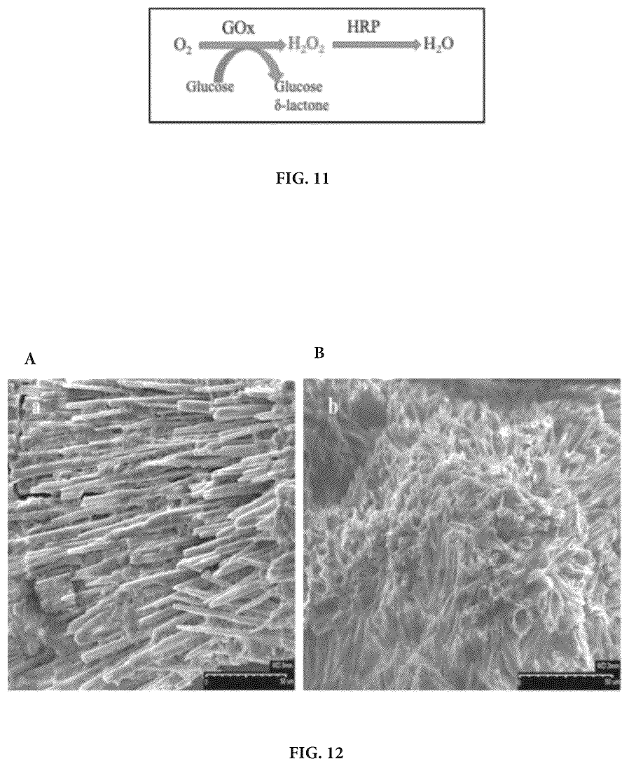

Diabetes mellitus is a major public health problem and a leading cause of death and disability in the world. It is a metabolic disorder in which the pancreas does not produce enough insulin. The diagnosis and management of this disease requires every-day monitoring of blood glucose levels. Different methods and enzymes have been used for this important task. Choosing the right enzyme to detect and monitor common diseases such as diabetes has become significant to development of an inexpensive and portable device. Development of a fast sensitive and reliable biosensor to detect glucose is imperative. Electrochemical biosensors, as biological devices, will become ever more prevalent and integral to medical as well as industrial and environmental applications. The enzymes most commonly used in currently available biosensors are glucose oxidase (GOx) and horseradish peroxidase (HRP) (FIG. 11).

However, HRP has several disadvantages because of the high cost of the enzyme, its limited reaction conditions, and its low catalytic effect to reduce hydrogen peroxide (H₂O₂) (Wu et al., 2011). In contrast, manganese peroxidase (MP) is known as a strong oxidizing enzyme (Singh et al., 2013) that can be used in a variety of applications. We introduced a new enzymatic biosensor using recombinant plant produced MP (PPMP). Our study shows that this recombinant manganese peroxidase (PPMP) has activity in detecting H₂O₂ as low as 1.3 μM (Izadyar et al., 2019). Here we outline how we will use this PPMP for a biosensor to detect glucose (Wang et al., 2014). In embodiments, we are applying this new enzyme with new nanoparticles to build an electrode by electrodeposition assembly using electrochemical techniques. We also will use conductive polymers (CPs) in this biosensor construction to stabilize the membrane, which will help us miniaturize the biosensor for real life applications.

Enzymatic sensor. Here we will validate a cost beneficial, sensitive, selective, and robust electrochemical biosensor to measure glucose. Non-enzymatic sensors for detection of glucose provide unavoidable disadvantages such as slow response time, irreproducibility and less stability over time. In enzymatic electrochemical glucose biosensors, the enzyme interacts with glucose, which leads to charge transfer to the electrode surface and provides good selectivity (Marquez et al., 2017). However, poor stability due to environmental effects can reduce the long-term accuracy of the biosensor. Therefore, current research and development of stable sensors with active enzymes for diabetes management is very important and is showing rapid progress. Horseradish peroxidase (HRP) with GOx are enzymes used to detect glucose (Yang et al., 2017) although they exhibit several disadvantages including high cost and limited reaction conditions. Other limitations for enzyme sensing such as pH, ionic strength, temperature or light can affect the activity of the enzyme. We will resolve these problems by using an alternative enzyme, a fungal manganese peroxidase (MP), which is produced in the cost-effective plant-based production system (Clough et al. 2006). Our research team has promising results showing that PPMP will be a new enzyme for the creation of selective and sensitive electrochemical biosensors.

Results

We fabricated a new enzymatic biosensor using recombinant plant produced Mn peroxidase (PPMP) purified from corn grain to generate a facile, fast and cost-beneficial biosensor for electrochemical detection of hydrogen peroxide (H₂O₂). The PPMP-modified gold electrode (PPMP/Au) showed excellent amperometric detection for the selective and sensitive enzymatic detection of H₂O₂. A wide linear range of 0.005 to 2.5 mM, and a lower detection limit (LOD) of 1.3 μM (1.3×10⁻⁶ molL⁻¹) were observed. The modified electrode also showed an excellent electrochemical performance for determination of H₂O₂ in orange juice as low as 7.5 μM. Without wishing to be bound by theory, the modified gold electrode can be used for applications in bioassays, environmental chemistry and food chemistry. The electrode fabricated with enzyme in buffer (a) showed highly order structure compared to the electrode treated with buffer only (b), indicating that the enzyme adhered in an orderly manner to the Au (FIG. 12).

These results showed that this PPMP is a significantly improved enzyme over HRP for creation of a selective and sensitive electrochemical biosensor (Izadyar et. al., 2019). The gene for the enzyme (Genbank accession #: L29039) was cloned from the white rot fungus. Phanerochaete chrysosporium and expressed recombinantly in corn grain (Clough et al., 2006). Therefore, not only does it show superior selectivity and sensitivity over HRP, the cost of production is significantly lower in the corn production system.

A. Magnitude of the Issues and the Relevance to Stakeholders:

Magnitude of the issues: During the past five decades, glucose has been one of the most tested analytes for millions of diabetes sufferers around the world, capturing about 85% of the entire biosensor market (Wang, 2008; Anderson et al., 2010). Electrochemical biosensors with high sensitivity, real time detection of sample, fast response, low cost, simplicity, good efficiency and high specificity of biological recognition processes are highly critical (Heineman et al., 2010); Bard et al., 2001; Alvau et al., 2018; Qi et al. 2014). During the past few decades, glucose monitoring in human blood has been growing (Klonoff et al., 2012). Glucose monitoring is also utilized in the food and chemical industries (Huang et al., 2016; Mason et al., 2016). Electrochemical sensors were chosen for glucose measurements because of their sensitivity and wide range of detection (μM to mM), (Makararn et al., 2014). Biosensors have been modified with conductive polymers, enzymes, and nanomaterials (Li et al., 2017). Enzyme-sensing is most prominent because of its high sensitivity, selectivity, and fast response time. Enzymes act as biocatalysts for biological processes in living organisms. Development of enzymes has become a significant part of the detection process for important compounds such as glucose. Nevertheless, several challenges in the achievement of accurate and reliable glucose monitoring remain: 1) technical improvements in glucose biosensors and 2) standardization of the analytical goals for their performance.

Stakeholder relevance: Electrochemical biosensor devices are used to detect and monitor glucose levels multiple times per day through finger sticks or with continuous monitoring, which is available for those with good insurance. The continuous sensors are expensive and only last for a maximum of 10 days before they become inaccurate and fouled with human fluids. Aspects of this invention resolve these problems by using more robust enzymes and anti-fouling polymers in the construction of the sensor.

Objectives

Here, we will use for the first time the PPMP enzyme, which has excellent electrocatalytic effect, to detect glucose. Also, to overcome the electron transfer issue between the electrode surface and protein we will use a nanoparticle-based solid electrode. Nanomaterials are used for improving this electron transfer limitation due to excellent conductivity, high catalytic activity, increasing the number of active sites on the surface and good adsorption of the analyte. Furthermore, we will examine for the first time the nanoparticle borophene, a molecule with significant transformative power. Our study with borophene will lead towards use of unique materials for biosensors. Here, we will entrap PPMP along with GOx in conductive polymers at a very thin layer of nanoparticles deposited on the solid electrode. Without wishing to be bound by theory, the results from this modified solid electrode will have applications in bioassays and food chemistry.

Without wishing to be bound by theory, an electrochemical sensor based on PPMP can be developed that is 5-fold more sensitive than the HRP-based sensor and that can resist fouling for 50% longer. Objectives to validate this:
1. Generate a two-layered electrochemical biosensor membrane applying Gox with an electrocatalytic enzyme, PPMP, via crosslinking.
2. Enhance the sensitivity of the biosensor using nanoparticles.
3. Apply an antifouling polymer during biosensor modification to enable real world measurements.

Methods:

Project Activities:

Objective 1: Generate a two-layered electrochemical biosensor membrane applying GOx with an electrocatalytic enzyme, PPMP, via crosslinking.

Rationale: Electrochemical biosensor modification using GOx with PPMP is a truly innovative approach, which for the first time uses this recombinant enzyme to act as a biocatalyst for detecting glucose. Application of the enzymes in a two-layered fashion will maximize the interaction of the enzymes with the analytes.

Techniques: Au/Gox-PPMP Electrode Performance to Detect Glucose. The recombinant plant produced MP (PPMP) can efficiently catalyze the oxidation of H₂O₂ into H₂O (Izadyar et. al., 2019). The steady-state amperometric response of the modified electrode was validated, with H₂O₂ from 0.005 to 2.5 m concentration under deoxygenated conditions using ultra-high purity (UHP) nitrogen gas before H₂O₂ was added and an inert atmosphere being created above the solution by passing N₂ gas over it (the standard deoxygenated solution). Aliquots of H₂O₂ were successively injected into the solution with a constant interval (50 s). The current-time response plot showed that the biosensor exhibited a rapid and sensitive response to the change of H₂O₂ concentration with an obvious change in current response. The sensor reached steady-state-current within a few seconds, indicating a fast and good electron transfer electrocatalytic performance of the PPMP immobilized on the Au electrode with a correlation coefficient of 0.998 and a lower limit of detection of about 1.3 μM. It can be noted that the PPMP/Au electrode shows stability in the solution for ˜3200.0 s (second) with a wider linear range.

Thus, the PPMP and GOx were a logical pair for constructing the electrochemical glucose biosensor. The gold working electrode (5 mm in diameter) was polished with an alumina paste slurry (0.3 and 0.05 μm) on a polishing pad (Buehler, Ltd., Lake Bluff, IL). A polished Au electrode was sonicated in ethanol for 15 min and in deionized water three times for 15 min. The electrode surface was then coated with 10 μL of the optimized enzyme solutions containing PPMP (40 mg mL⁻¹), bovine serum albumin (100 mg mL⁻¹) and glutaraldehyde (0.10%, 50 μL mL⁻¹) onto the Au electrode, and allowed to air dry.

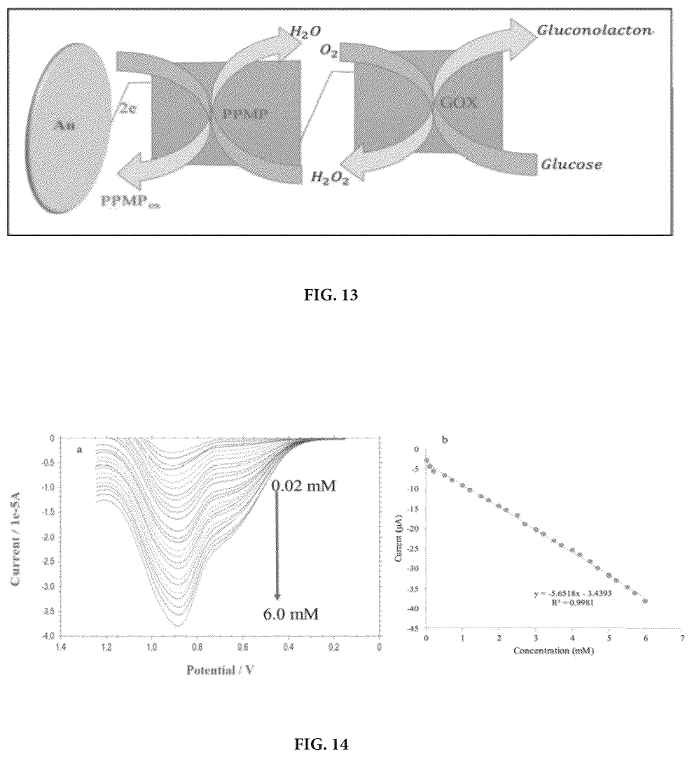

In the next step, 10 μL of the optimized enzyme solution containing GOx (30 mg mL⁻¹), bovine serum albumin (100 mg mL⁻¹) and glutaraldehyde (0.10%, 50 μL mL⁻¹) were cast onto the PPMP-Au electrode based on the cross-linking reaction and dried at 4° C. (FIG. 13).

Several methods can be employed to determine the functionality, specificity, selectivity and sensitivity of an electrode. These include Linear Sweep Voltammetry (LSV), Amperometry, and Cyclic Voltammetry (CV). The advantages of each are shown in Table 3.

The electrochemical performances of the Au/GOx-PPMP modified electrode were investigated by linear sweep voltammetry (LSV). The results of the linear sweep voltammogram are shown in FIG. 14. In LSV, the two most important characteristics are Ep, the voltage at which the current peaks, and ip, the value of the current at that point (Bard et al., 2001). The current is proportional to the concentration of glucose. The limit of detection (LOD) of 0.0034 mM was calculated for SIN=3 and S=(3× std dev)+blank (Harris, D. C. 2020; pp 83-89). The R² values of the regression lines that fitted to the calibration curves were 0.9981.

TABLE 3
Electrochemical methods

| | Electrochemical | |
|---|---|---|
| Method | performance measure | Advantage of Method |
| Linear sweep voltammetry | In linear sweep voltammetry | LSV can determine the rate |
| (LSV) | (LSV) a fixed potential range | of the electron transfer |
| | is employed and the | reaction(s), evaluate the |
| | oxidation or reduction of a | chemical reactivity of the |
| | molecule is recorded. LSV is | electroactive species (here |
| | a three-electrode setup | enzyme (PPMP)), identify |
| | consisting of a working | unknown species, and |
| | electrode, a counter | determine the concentration |
| | electrode, and a reference | of solutions. |
| | electrode. In embodiments, | |
| | the gold electrode (working) | |
| | is one of the electrodes at | |
| | which the oxidation reaction | |
| | occurs. The current is a direct | |
| | measure of the rate at which | |
| | electrons are being | |
| | exchanged through the gold | |
| | electrode-solution interface | |
| | which contains glucose and | |
| | exhibits a peak. | |
| Cyclic Voltammetry (CV) | CV can be used for reversible | Cyclic voltammetry has the |
| | reactions to find information | same advantages as LSV but |
| | about the forward reaction | can provide information |
| | and the reverse reaction. | about both oxidation and |
| | | reduction reactions. |
| | | Although CV is applicable to |
| | | most cases where LSV is |
| | | used, in some of our glucose |
| | | evaluations, because the |
| | | reaction is irreversible, |
| | | cyclic voltammetry will not |
| | | give any additional data that |
| | | linear sweep voltammetry |
| | | can give us. |
| Amperometry | We will apply amperometry | This method is very selective |
| | as an electroanalytical | and offers the ability to |
| | technique that involves the | distinguish specific species |
| | application of a constant | between a number of |
| | oxidizing potential to the | electroactive species in |
| | modified gold electrode and | solutions by the applied |
| | the subsequent measurement | specific potential to choose |
| | of the resulting steady-state | just the target analyte (e.g. |
| | current. Without wishing to | glucose) in the solutions. |
| | be bound by theory, the | Due to the sensitivity and |
| | magnitude of the measured | selectivity of this technique, |
| | current is proportional to the | amperometric biosensors are |
| | concentration change of the | widely used for clinical |
| | glucose. | monitoring. |

Amperometric Technique. The amperometric technique in comparison with cyclic voltammetry and LSV shows higher sensitivity and selectivity, favoring use of the amperometric biosensor. Additionally, an important requirement for a biosensor is its long-term stability. However, amperometric biosensors can have two disadvantages which affect their life span: enzyme leakage and enzyme denaturation. In fact, amperometric biosensors are able to combine the robustness of electrochemical techniques with the specificity of biological recognition processes, favoring amperometric sensor use. Nevertheless the sensing system must have a stable membrane, which is important in biosensor structure. Additionally amperometric methods can offer real time detection of glucose and facile mass production (Rivera et al. 2015). Without wishing to be bound by theory, embodiments as described herein will stabilize the membrane while maintaining sensitivity.

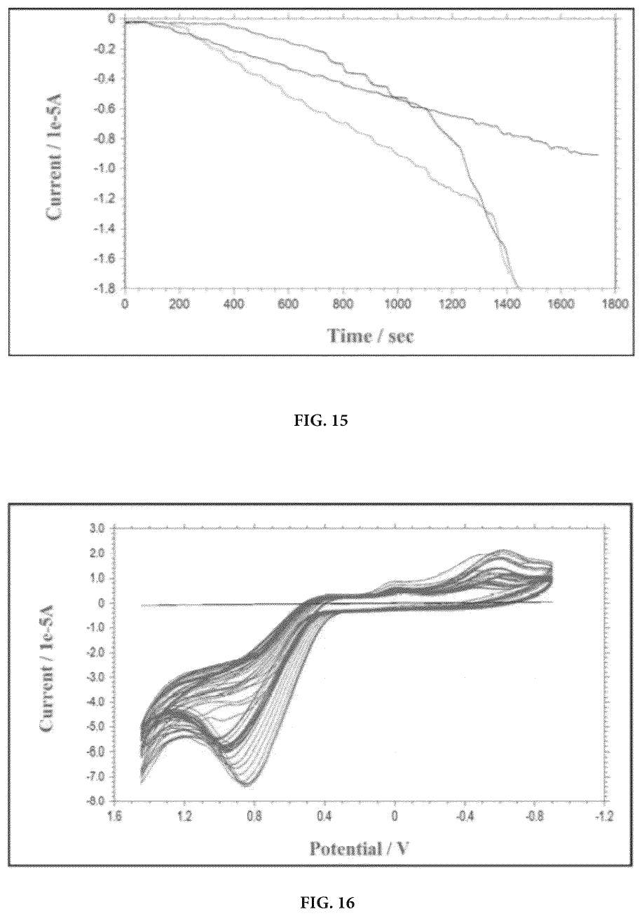

Amperometric Responses of the PPMP/Au Electrode to Glucose. Biosensor stability was validated by comparing the slopes of calibration curves. We applied this technique under the same conditions as LSV (oxygen purging, PBS pH 7.0 solution upon the successive addition of glucose while stirring) to detect the steady-state amperometric response of the modified electrode. We applied different potentials to the amperometric response. The amperometric responses of the PPMP modified gold electrode evaluated at applied potentials of 0.75, 0.85 and 0.90 V are shown in FIG. 15. The solutions measured at a constant time interval did not reach steady state which, without wishing to be bound by theory, depends on the morphology of the of the membrane on the Au electrode. This example will provide membrane modifications to achieve sustainable results.

Outcomes.

Without wishing to be bound by theory, we will optimize the immobilization parameters and subsequently validate the activity of the PPMP mixed with Gox using an amperometric technique. Also, we will validate the specific parameters that are needed to generate the most effective activity of the electrode.

To validate the efficiency of the embodiments described herein, we will use amperometry with a rotating modified PPMP. GOx/Au biosensor in order to detect lower concentrations of glucose. We will display amperometric responses of the PPMP. GOx/Au biosensor with the successive addition of glucose in PBS pH 7.0 under O₂ purging at a potential.

Objective 2: Enhance the sensitivity of the biosensor using nanoparticles.

Nanomaterials with unique properties can provide a larger surface area for deposition, and greater sensitivity and conductivity because more enzyme is present. We will fabricate and characterize new nanoparticles, graphene and borophene. The morphology of the biosensor at each step will be investigated using scanning electron microscopy (SEM) to ensure the surface topography and composition of membrane are ordered.

Techniques

Nanoparticle-based working electrode. Nowadays, demands for quick and economical health measurement as well as a focus on a green environment require more expanded application of nanotechnology-based sensors. Nanosensing using electrochemistry is a unique technology, which is cost-effective, selective, sensitive, and can be used for multiple analyses. Nanoparticles such as graphene oxide (GO) with unique mechanical strength, high specific surface area, fast electron transfer effect, chemical stability (Yan et al., 2013: Izadyar et al., 2018), excellent conductivity and a small band gap are promising for sensing applications which are beneficial in electrochemical sensor techniques.

The Borophene-Based Working Electrode. Borophene is a single layer of boron atoms (Wang, et al., 2019) and can facilitate creating new nanoelectronics devices. It was first synthesized in 2015 (Mannix, et al., 2015), and scientists have since discovered that it is super-strong, super-flexible, and a superconductor, even stronger and more flexible than graphene. The experimental and theoretical studies on borophene show some unique physical and chemical properties. or instance, high capacity and excellent electronic conductivity can be achieved with borophene in a vast number of applications. Nevertheless, experimental investigation of the physical properties of borophene remains an active area especially in sensor technology. Here, we will validate this new nanoparticle as a comparable approach to a graphene-based biosensor.

Nanoparticle-Based Sensors Using Electrochemical Techniques. To validate Boron deposition, a gold electrode was coated with drop casting and electrodeposition of boron nanoparticles. FIG. 16 shows electrodeposition on the gold (Au) electrode of 40 mg of boron nanoparticle (BN) distributed in 25 ml of dimethyl formamide (DMF) solvent, with sixty cycles under 2 gas purging system at scan rate 0.1 V/s. Electrochemical behavior was validated for each electrode by cyclic voltammetry (CV) experiments, which are shown by red lines in the cathodic and anodic scan. Cyclic voltammetry (CV) is a powerful electrochemical technique employed to validate the reduction and oxidation of molecular species.

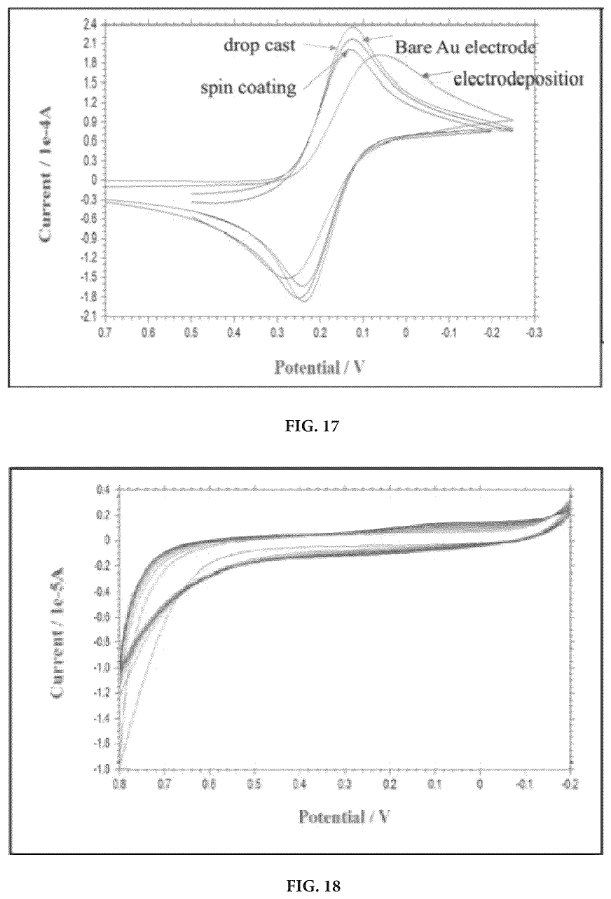

Cyclic voltammetry (CV). Cyclic voltammetry is an electroanalytical technique for electroactive species. We applied 5 Mm FeIII(CN)₆³⁻/FeII(CN)₆⁴⁻. This redox couple exhibits a reversible reaction without any complications of proceeding or post chemical reactions. Thus, the ferricyanide/ferrocyanide couple has been applied as an electrochemically reversible redox system to study Au/BN electrodeposited electrode, the Au/BN drop casting electrode, and the Bare electrode. The result indicates that an ohmic potential drop is negligible using the B based electrode and charge transport through this B modified electrode shows a good electrochemically reversible CV (FIG. 17).

Linear sweep voltammetry (LSV) and amperometry using PPMP, Gox Au/NPs. LSV and amperometry will be employed using a PPMP. Gox Au modified graphene or borophene electrode. We will validate the ohmic potential drop is negligible using the NP-based electrode and whether charge transport through this NP-modified electrode shows a better electrochemical response. Moreover, the selectivity will be controlled by control potential in the amperometry technique.

Outcomes. Without wishing to be bound by theory, the performance of nanoparticle-based biosensors will improve through the introduction of nanotechnology. We are highly experienced in electro-polymerization of nanoparticles such as graphene (Izadyar et al., 2018) and synthesis and application of conductive polymers (Izadyar et al., 2016; Izadyar et al., 2014) and biosensor development (Izadyar et al., 2019).

Borophene is a new nanoparticle in the biosensor area. The morphology of modified electrodes in each step will be validated by SEM. SEM images can provide information about the porosity on the electrode surface. Therefore, we will validate the optimum number of nanoparticle electrodeposition cycles based on these SEM observations combined with activity monitoring. Moreover, the electropolymerized conductive polymer performance with different cycles and scan rate will be validated using SEM images to obtain more homogenously covered surfaces of the biosensor.

Objective 3: Apply an antifouling polymer during biosensor modification to enable real world measurements.

We will validate the challenges of conductive polymers (CP) and nanomaterials (graphene and borophene) used together in the construction of biosensors in terms of practical application. Without wishing to be bound by theory, polymeric membrane can be used for protein adsorption, which minimizes the active electrode surfaces and fouls the biosensor. We will avoid loss of enzyme activity through deposition of the CP matrix. We will apply nontoxic and hydrophilic polymers such as Polypyrrole (PPy), poly(ethylene glycol) (PEG), and 4-ethylene dioxythiophene (PEDOT), which induce the release of the adsorbed proteins and cells at the surface of the biosensor during glucose monitoring.

Techniques: Conductive polymers (CPs) offer a group of effective features to biosensors depending on the polymers and the fabrication/modification methods. Without wishing to be bound by theory, conductive polymers can act as an electrical carrier. Conductive polymers are competitive sensing materials for biological sensing applications due to inherent electrical conductivity, which is closely connected to their charge transfer rate and electrochemical redox efficiency. Moreover the ease of functionalization of these materials through the doping process can also play an important role in developing new applications, for example, PPy and PEDOT (poly(3,4-ethylenedioxythiophene), and polystyrene sulfonate (PEDOT)) (Izadyar et al., 2016). Here, we will validate different conductive polymers using electro-polymerization techniques to immobilize these newly paired enzymes.

Electropolymerization of Pyrrole with GOx and PPMP on the Au electrode. Pyrrole polymerization was carried out by cyclic voltammetry and its voltammogram is shown in FIG. 18. During polymerization the polymer growth and the cathodic peak Epc were observed at 0.1 V during the initial backward scan.

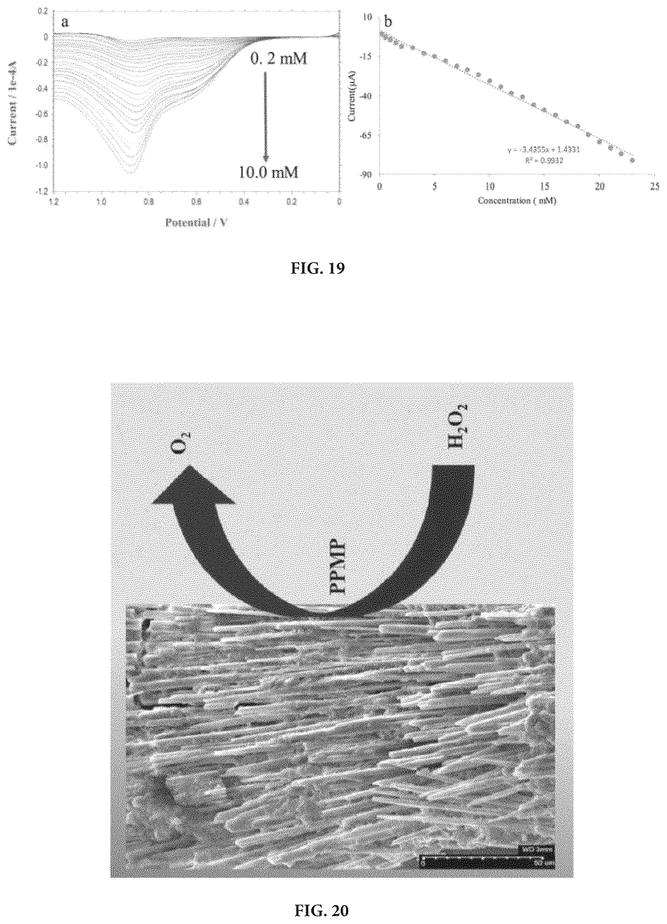

The electrochemical performances of the Au/Ppy-GOX-PPMP modified electrode were validated by Linear sweep voltammetry. The result of the linear sweep voltammogram is shown in FIG. 19. The lower limit of detection of the sensor was found to be 0). 16 mM which is higher than a two-layered electrochemical biosensor membrane with a 0.0034 mM detection limit.

Amperometry using Au/NPsl CPs-GOX-PPMP. An amperometry technique will be employed using PPMP. GOx Au modified graphene or borophene electrode for monitoring glucose. For the biosensor to be applicable in-situ and practically, embodiments can use the immobilization of enzymes in a polymer. This will also reduce the detection limit and increase sensitivity of the biosensor. Without wishing to be bound by theory, we will develop a biosensor in the form of a combination of nanoparticles (NPs), conductive polymers (CPs) and enzyme extracted from corn Au/NPs/CPs-GOX-PPMP. Different antifouling polymers will be examined as well as the number of cycles during polymerization. CP-nanomaterials-based biosensors have been used in energy, environmental, and biomedical applications and exhibit acceptable performance while sensing target molecules. Without wishing to be bound by theory, embodiments herein comprise the use of new CP and nanomaterials on fabrication and modification of biosensors. Amperometric electrochemical technology bas established several commercial glucose meters (O'Kane et al., 2009; Oliver et al., 2009) to help patients manage blood glucose in routine testing. In this work, we will validate the fabrication and characterization of a new glucose biosensor based on an amperometric method that will have superior characteristics. Aspects of the invention comprise an affordable and highly active enzyme “PPMP”. The nanoparticle-based electrode will be electrochemically fabricated through a deposition that makes an active and larger surface area, thus enhancing the sensitivity of the biosensor. Antifouling and stability of the glucose biosensor will have improved performance using CPs.

Outcomes. Without wishing to be bound by theory, embodiments herein will be able to monitor glucose using whole blood, plasma, and serum samples. (McMillin et al., 1990). We are highly experienced in sensor development as shown in our previous work (Izadyar et al. 2016). Different antifouling polymers in this aim will be validated as well as the number of cycles during polymerization. CP— nanomaterials-based biosensors are applicable in energy, environmental, and biomedical applications and exhibit acceptable performance while sensing target molecules. Without wishing to be bound by theory, aspects of the invention will provide a technique using new CP nanomaterials on fabrication and modification of biosensors. Amperometric electrochemical technology has established several commercial glucose meters (O'Kane et al., 2009; Oliver et al., 2009) to help patients manage blood glucose routine testing. In this work, we will validate the fabrication and characterization of a new glucose biosensor based on an amperometric method that will have superior characteristics. Aspects of the invention comprise an affordable and active enzyme “PPMP”. The nanoparticle-based electrode will be electrochemically fabricated through a deposition that makes an active and larger surface area, thus enhancing the sensitivity of the biosensor. Anti-fouling and stability of the glucose biosensor will have improved performance using CPs. Without wishing to be bound by theory, aspects of the invention will be able to monitor glucose using whole blood, plasma, and serum samples. (McMillin et al., 1990).

In embodiments, we will apply an antifouling polymer. In embodiments, we will use simultaneously use nanoparticles and immobilization of the enzymes into the polymeric film simultaneously.

Aspects of the invention can include: 1) validating the mechanisms and performance of nanoparticle-based biosensors combined with conductive polymers, which are significant steps leading to miniaturization and mass production of biosensors because of improvements in the stability of the membrane and increases in the life span: 2) validating the selective, sensing and recognition mechanisms of glucose biosensors, which are classified as emerging biotechnology for micro-level sensing. The applicability of the modified electrode described herein will be validated to monitor real samples such as interstitial fluid glucose (JG) and blood glucose: having a positive impact for diabetes sufferers.

Evaluation Plan.

Research Activities: The sensing and recognition mechanisms of microbiosensors such as electrochemical type devices (Su et al., 2011) are classified as emerging biotechnology for miniaturized sensing mechanisms. Therefore, validating the newly constructed biosensors and their mechanisms is very important. The details of biotechnological approaches to a miniaturized biosensor to detect glucose have to be specifically designed for subcutaneous tissue conditions in order to avoid loss of sensitivity over time. In embodiments, miniaturization of the polymeric membrane biosensor on an ultramicroelectrode can be used for glucose monitoring in a small volume such as in human tissue. Further, embodiments will comprise ultramicroelectrode fabrication using the laser-assisted pulling method because of the excellent control of the electrode geometry and its high repeatability (Zhang et al., 2007). In embodiments, we will use the laser-based micropipette puller system in the fabrication of the ultramicroelectrode and a graduate student in the lab will perform these experiments.

### REFERENCES CITED IN THIS EXAMPLE

Alvau M D, Tartaggia S. Meneghello A. Casetta B. Calia G, Serra P A, Polo F, Toffoli G. 2018. Enzyme-Based Electrochemical Biosensor for Therapeutic Drug Monitoring of Anticancer Drug Irinotecan. Anal. Chem. 90:6012-6019.
Bard A J, Faulkner L R. 2001. Electrochemical Methods: Fundamentals and Applications: 2^{nd} ed.; John Wiley & Sons: New York, p 339.
Bruen D, Delaney C, Florea L, Diamond D. 2017. Review, Glucose Sensing for Diabetes Monitoring: Recent Developments. Sensors 17: 1866-1887.
Gao W, Emaminejad S, Nyein H Y Y, Challa S, Chen K, Peck A. 2016. Fully integrated wearable sensor arrays for multiplexed in situ perspiration analysis. Nature. 529(7587): 509-14.
Clough, R. C., Pappu, K., Thompson, K., Beifuss, K., Lane, J., Delaney, D. E., Harkey, R., Drees, C., Howard, J. A. and Hood, E. E. (2006) Manganese peroxidase from the white-rot fungus Phanerochaete chrysosporium is enzymatically active and accumulates to high levels in transgenic maize seed. Plant Biotechnol. J. 4:53-62.
Damodaran V B, Murthy N S. 2016. Bio-inspired strategies for designing antifouling biomaterials. Biomater. Res. 20: 18.
Harris, D. C. Quantitative Chemical Analysis, 8th ed.; W. H. Freeman and Company: New York, 2020; pp 83-89.
Hui N, Sun X, Niu S, Luo X. 2017. PEGylated polyaniline nanofibers: Antifouling and conducting biomaterial for electrochemical DNA sensing. ACS Appl. Mater. Interfaces 9:2914-2923.
Izadyar A, Tran U, Hood E E. 2019 “Recombinant Mn Peroxidase from Corn Grain Has an Excellent Electrocatalytic Effect in a Designed Amperometric Biosensor to Detect Hydrogen Peroxide at Low Concentrations” ACS Sustainable Chem. Eng. 2019, 7, 194 34-19441. DOI: 10.1021/acssuschemeng.9b04216.
Izadyar A, Hershberger J C, Robert R. 2018. Voltammetric Assessment of Ions Transfer at Ionophore-Graphene Based Polymeric Membranes. Electroanalysis, 30(11):2580-2583.
Izadyar A, Al-Amoody F, Ranawaka Arachchige D. 2016. Ion transfer stripping voltammetry to detect Nanomolar concentrations of Cr (VI) in drinking water” J. Electroanalytical. Chemistry 782: 43-49.
Izadyar A, Ranawaka Arachchige D, Cornwell H, and Hershberger J L. 2016. Ion Transfer Stripping Voltammetry for the Detection of Nanomolar Levels of Fluoxetine, Citalopram, and Sertraline in Tap and River Water Samples. Sensors and Actuators B: Chemical 223:226-233.
Marquez A, Jimenez-Jorquera C, Dominguez C, Mufioz-Berbel X. 2017. Lector depositable alginate membranes for enzymatic sensors: An amperometric glucose biosensor for whole blood analysis. Biosens. Bioelectron. 97: 136-142.
McMillin J M. 1990. Blood Glucose. In Clinical Methods: The History, Physical and Laboratory Examinations, 3rd ed.; Walker, H. K., Hall, W. D., Hurst, J. W., Eds.; Butterworth-Heinemann: Boston, M A, USA.
O'Kane M J, Pickup J. 2009. Self-monitoring of blood glucose in diabetes: Is it worth it? Ann. Clin. Biocbem. 46: 273-282.
Park C S, Changsoo Lee C, Kwon O S. 2016. Review, Conducting Polymer Based Nanobiosensors” Polymers 8: 249.
Qi P, Zhang D, Wan Y. 2014. Development of an Amperometric Microbial Biosensor Based on Thiobacillus thioparus Cells for Sulfide and Its Application to Detection of Sulfate Reducing Bacteria. Electroanalysis 26: 1824-1830.
Ronkainen N J, Halsall H B, Heineman W R. 2010. Electrochemical biosensors” Chem Soc Rev. 39(5): 1747-63.
Rubinstein I, Bard A J (1980) Polymer films on electrodes. 4. Nation-coated electrodes and electrogenerated chemiluminescence of surface-attached tris (2,2′-bipyridine) ruthenium (2+). Journal of the American Chemical Society 102: 6641-6642.
Singh R, Grigg J C, Qin W, Kadla J F, Murphy M E P, Lindsay D, Eltis L D. 2013. Improved Manganese-Oxidizing Activity of DypB, a Peroxidase from a Lignolytic Bacterium. ACS Chem. Biol. 8: 700-706.
Su L, Jia W, Hou C, Lei Y. 2011. Microbial biosensors: a review. Biosens. Bioelectron. 26:1788-1799.
Torrent M, Llompart B, Lasserre-Ramassamy S, Llop-Tous I, Bastida M, Marzabal P, Westerholm-Parvinen A, Saloheimo M, Heifetz P B, Ludevid M D. 2009. Eukaryotic protein production in designed storage organelles. BMC Biol 7: 5.
Updike S J, Hicks G P. 1967. The enzyme electrode. Nature 214: 986.
Wang J. 2008. Electrochemical Glucose Biosensors. Chem. Rev. 108: 814-825.
Wang J, Myung N V, Yun M, Monbouquette H G. 2005. Glucose oxidase entrapped in polypyrrole on high-surface-area Pt electrodes: a model platform for sensitive electroenzymatic biosensors. Journal of Electroanalytical Chemistry 575: 139-146.
Wang W, Xie Y, Wang Y, Du H, Xia C, Tian F. 2014. Glucose biosensor based on glucose oxidase immobilized on unhybridized titanium dioxide nanotube arrays. Microchim. Acta, 181: 1325-1331.
Wu P, Cai Z, Chen J, Zhang H, Cai C. 2011. Electrochemical Measurement of the Flux of Hydrogen Peroxide Releasing from RAW 264. 7 Macrophage Cells Based on EnzymeAttapulgite Clay Nanohybrids. Biosens. Bioelectron. 26:4012-4017.
Yang H, Gong C, Miao L, Fugang X. 2017. A Glucose Biosensor based on Horseradish Peroxidase and Glucose Oxidase Co-entrapped in Carbon Nanotubes Modified Electrode. Int. J. Electrochem. Sci., 12: 4958-4969.
Zhang B, Galusha J, Shiozawa P G, Wang G, Bergren A J, Jones R M, White R J, Ervin E N, Cauley, C C, White H S. 2007. Bench-Top Method for Fabricating Glass-Sealed Nanodisk Electrodes, Glass Nanopore Electrodes, and Glass Nanopore Membranes of Controlled Size. Anal. Chem. 79: 4778-4787.

### Example 5—a Bienzymatic Amperometric Glucose Biosensor Based on Using a Recombinant Mn Peroxidase from Corn and Glucose Oxidase with a Nation Membrane

Abstract

Using a recombinant enzyme derived from corn and a simple modification, we fabricated a facile, fast, and cost-beneficial new biosensor to measure glucose. The Nafion™/Plant Produced Mn Peroxidase (PPMP)—glucose oxidase (GOx)—Bovine serum albumin (BSA)/Au electrode showed an excellent amperometric response to detect glucose. This biosensor can respond to a wide range of glucose. 20.0 μM-15.0 mM, and has a lower detection limit (LOD) of 2.9 μM. The reproducibility response using six electrodes is also very substantial and indicates high capability of this biosensor to detect glucose concentrations. Selectivity of this electrode was validated in an optimized experimental solution containing 10% diet green tea with citrus containing ascorbic acid (AA), and citric acid (CA) in a wide concentration of glucose at 0.02 to 14.0 mM with an LOD of 3.1 μM. We also applied linear sweep voltammetry (LSV) and this technique shows a wide range of glucose detection. The performance and strength of this new enzyme biosensor were the simplicity, wide linear ranges, sensitivities, selectivity, and low limits of detection. Without wishing to be bound by theory, the modified biosensor can be used for monitoring various biofluids.

Development of a Nano-Biosensor to Detect Glucose for Diabetics, Using Recombinant Manganese Peroxidase from Corn Grain.

Here provides a project that puts us at the forefront of one of the most important branches of agricultural chemistry: using enzymes to develop and modify a biosensor for diabetes. Importantly, these enzymes can be manufactured in a corn kernel biofactory. Embodiments herein comprise the miniaturization of a significantly improved glucose biosensor that has increased sensitivity and longevity for diabetes sufferers. Embodiments herein can be dependent on using enzymes produced from an agricultural crop.

An Amperometric Glucose Sensor Using Recombinant Mn Peroxidase and Glucose Oxidase

Electrochemical sensor devices are used to detect and monitor glucose levels multiple times per day through finger sticks. The continuous sensors are expensive and only last for a maximum of 10 days before they become inaccurate and fouled with human fluids. Embodiments herein resolve these problems by using more robust enzymes and anti-fouling polymers in the construction of the sensor. The enzymes used in sensors are glucose oxidase (GOx) and horseradish peroxidase (HRP). However. HRP has several disadvantages including its high cost, its limited reaction conditions and its low catalytic effect to reduce hydrogen peroxide (H₂O₂). Manganese peroxidase (MP) is a strong oxidizing enzyme that can be used in a variety of sensor applications. We introduced a new enzymatic biosensor using recombinant plant-produced MP (PPMP). This recombinant PPMP has activity in detecting H₂O₂ as low as 1.3 μM. Additional components that can increase longevity and sensitivity are conductive polymers (CPs) and nanoparticles. Here, we use PPMP and new conductive polymers together to validate a more sensitive and longer-lived biosensor to detect glucose, the next step in the process of validating a miniaturized sensor.

Amperometric Biosensor for Glucose Determination Based on a New Recombinant Mn Peroxidase from Corm Cross-Linked to a Gold Electrode

Abstract

Using a recombinant enzyme derived from corn and a simple modification, we fabricated a facile, fast, and cost-beneficial biosensor to measure glucose. The Nafion™/Plant Produced Mn Peroxidase (PPMP)—glucose oxidase (GOx)—Bovine serum albumin (BSA)/Au electrode showed an excellent amperometric response to detect glucose. This biosensor can responding to a wide range of glucose 0.0 μM-15.0 mM and has a lower detection limit (LOD) of 2.90 μM. The reproducibility response using six electrodes is also very substantial and indicates this biosensor can detect a wide range of 3.10±0.19 μM to 13.2=1.8 mM glucose concentration. Selectivity of this electrode was validated in an optimized experimental solution contains 10% diet green tea with citrus containing ascorbic acid (AA), and citric acid (CA) in a wide concentration of glucose at 0.02 to 14.0 mM with an LOD of 3.10 μM. Reproducibility was also validated using 4 electrodes in this sample and shows notable results in the wide concentration range of 3.35±0.45 μM to of 13.0±0.81 mM. We also used other voltammetry methods to validate this biosensor. We applied linear sweep voltammetry (LSV) and this technique shows a wide range of 0.10-15.0 mM to detect glucose with a lower detection limit of 19.5 μM. The performance and strength of this enzyme biosensor were the simplicity, wide linear ranges, sensitivities, selectivity, and low limits of detection. Without wishing to be bound by theory, the modified biosensor can be used for monitoring various biofluids.

Electrochemical Technique to Fabricate Glucose Biosensor Using Enzyme Extract from Corn

Using a recombinant enzyme derived from corn and a simple modification, we fabricated a facile, fast, and cost-beneficial biosensor to measure glucose. The Nafion™/Plant Produced Mn Peroxidase (PPMP)—glucose oxidase (GOx)—Bovine serum albumin (BSA)/Au electrode showed an excellent amperometric response to detect glucose. This biosensor can respond to a wide range of glucose—20.0 μM-15.0 mM and has a lower detection limit (LOD) of 2.90 μM. The reproducibility response using six electrodes is also very substantial and indicates this biosensor can detect a wide range of 3.10±0.19 μM to 13.2±1.8 mM glucose concentration. Selectivity of this electrode was validated in an optimized experimental solution contains 10% diet green tea with citrus containing ascorbic acid (AA), and citric acid (CA) in a wide concentration of glucose at 0.02 to 14.0 mM with an LOD of 3.10 μM. Reproducibility was also validated using 4 electrodes in this sample and shows notable results in the wide concentration range of 3.35±0.45 μM to of 13.0±0.81 mM. We also used other voltammetry methods to validate this biosensor. We applied linear sweep voltammetry (LSV) and this technique shows a wide range of 0.10-15.0 mM to detect glucose with a lower detection limit of 19.5 μM. The performance and strength of this enzyme biosensor were the simplicity, wide linear ranges, sensitivities, selectivity, and low limits of detection. Without wishing to be bound by theory, the modified biosensor can be used for monitoring various biofluids.

Electrochemical Technique to Fabricate Glucose Biosensor Using Enzyme Extract from Corn

Electrochemical sensor devices are used to detect and monitor glucose levels multiple times per day through finger sticks. The continuous sensors are expensive and only last for a maximum of 10 days before they become inaccurate and fouled with human fluids. We will resolve these problems by using more robust enzymes and anti-fouling polymers in the construction of the sensor. The enzymes used in sensors are glucose oxidase (GOx) and horseradish peroxidase (HRP). However. HRP has several disadvantages including its high cost, its limited reaction conditions, and its low catalytic effect to reduce hydrogen peroxide (H₂O₂). Manganese peroxidase (MP) is a strong oxidizing enzyme with promising potential in a variety of sensor applications. We introduced a new enzymatic biosensor using recombinant plant-produced MP (PPMP). Our recent study shows that this recombinant PPMP has activity in detecting H₂O₂ as low as 1.3 μM.

### Example 6 Amperometric Biosensor for Glucose Determination Based on Recombinant Mn Peroxidase from Corm (PPMP) Cross-Linked to a Gold Electrode

Outline
H₂O₂ Sensor using PPMP
Glucose Sensor using PPMP
Enhance the sensitivity and stability of the biosensor using nanoparticles and conductive polymers

Recombinant Mn Peroxidase from Corn (PPMP)

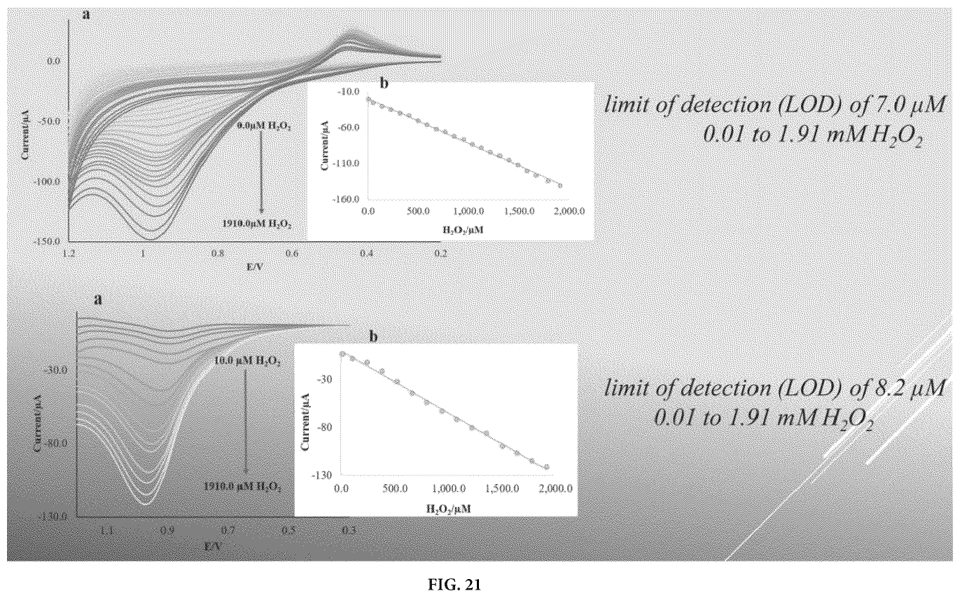

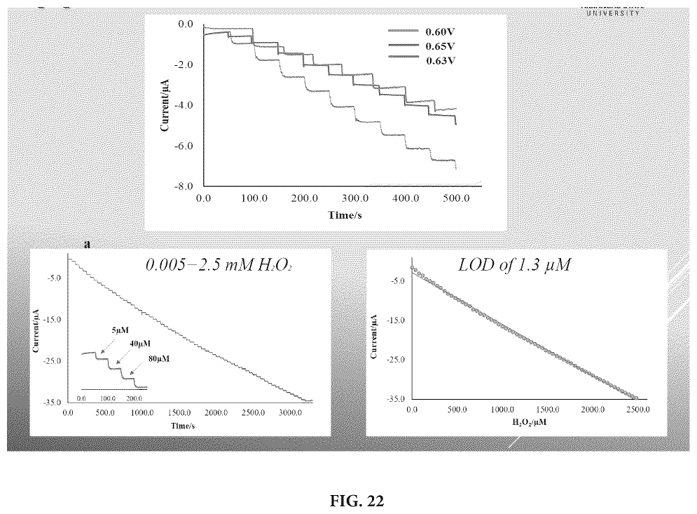

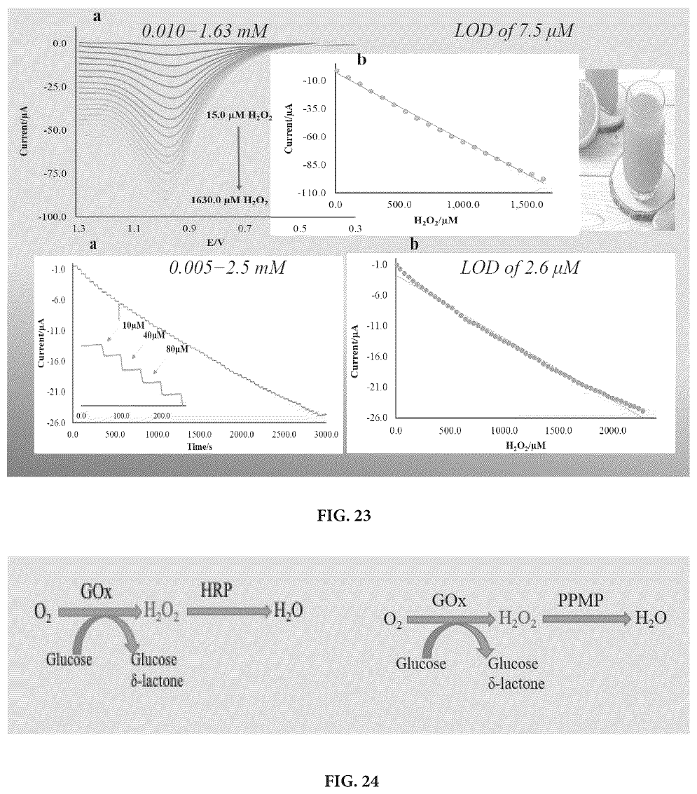

20 μL of solution (0.2 mM of enzyme in tartrate buffer pH 4.5) spin casting, in deoxygenated PBS/0.1 mM Mn²⁺ (FIG. 20)
Direct electrochemistry of the PPMP/Au electrode applying cyclic voltammetry (CV) and Linear Sweep Voltammetry (LSV) (FIG. 21)
Amperometric Responses of the PPMP/Au electrode to H₂O₂ (FIG. 22)
Selectivity Evaluation of the PPMP Biosensor in Orange Juice (FIG. 23)
Why Glucose (FIG. 24)

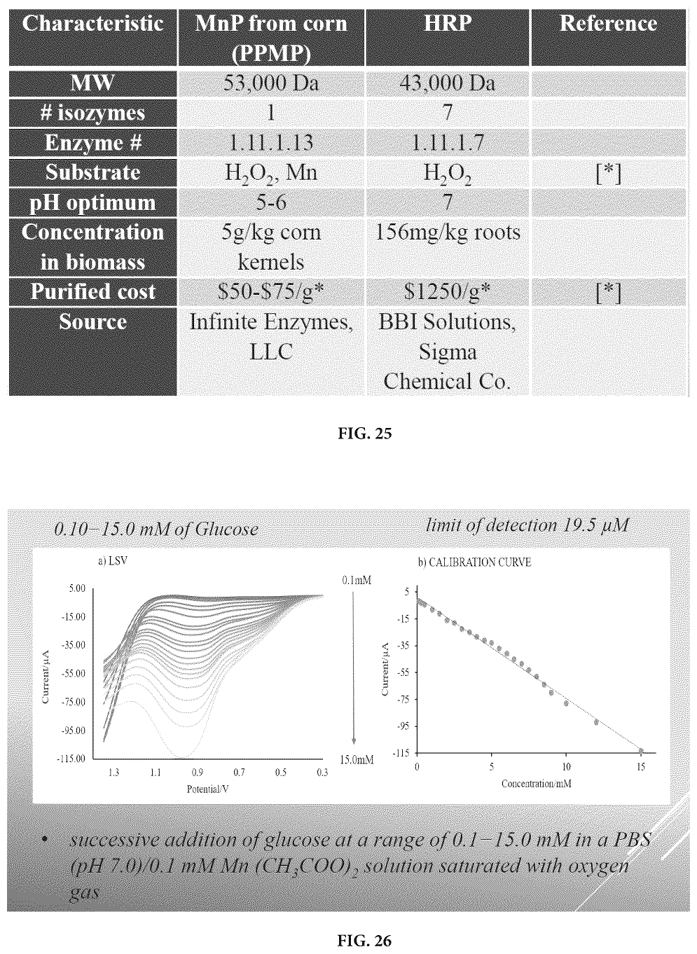

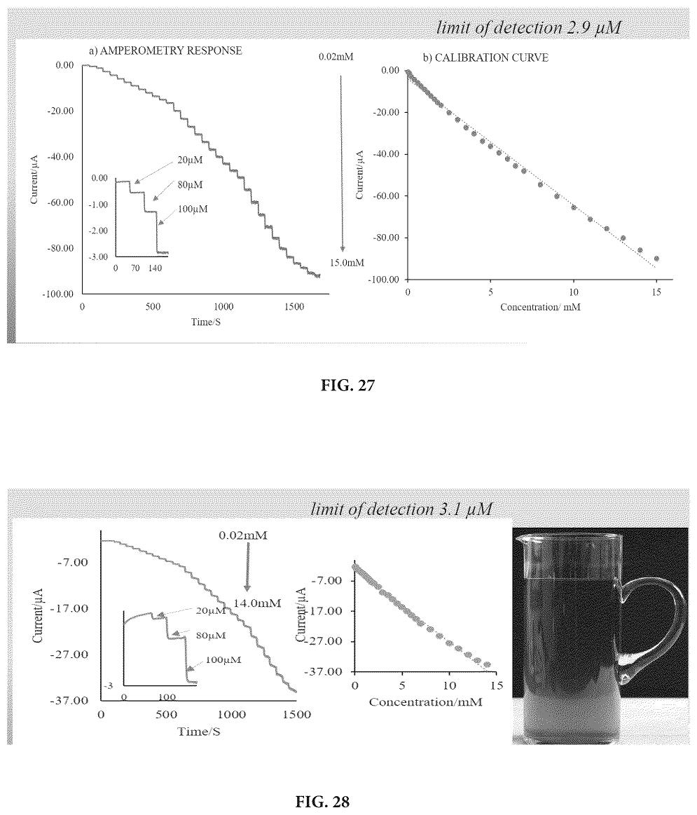

HRP has Several Disadvantages:
high cost of the enzyme
its limited reaction conditions
its low catalytic effect to reduce H₂O₂
GMP at scale, Cost comparison of PPMP and HRP (FIG. 25)
Linear Sweep Voltammetry (LSV) of the Nafion™/PPMP-Gox-BSA/Au Electrode (FIG. 26)
Amperometric Responses of the Nafion™/PPMP-Gox-BSA/Au Electrode to detect glucose (FIG. 27)
Selectivity of the Nafion™/PPMP-Gox-BSA/Au Electrode (FIG. 28)

10% diet green tea, contains green tea, CA, sodium hexametaphosphate, water, natural flavor, AA, phosphoric acid, aspartame, acesulfame potassium, calcium disodium, EDTA, and caramel color.
Morphology studies

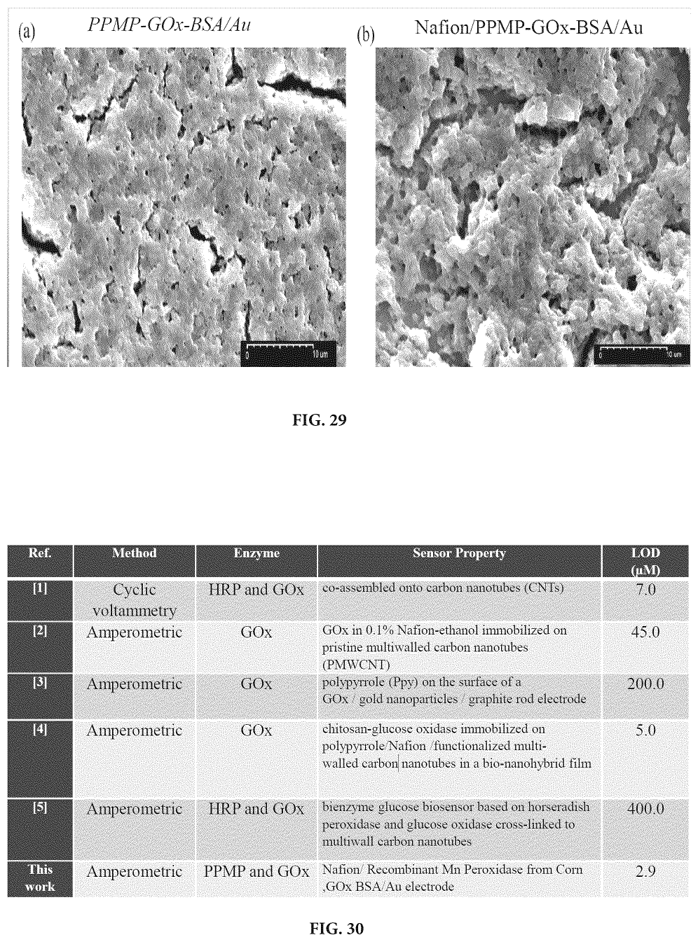

Scanning Electron Microscope (SEM) Images (FIG. 29)

FIG. 30 shows a comparison of glucose determination with differently modified electrodes
Enhance the sensitivity and stability of the biosensor using conductive polymer

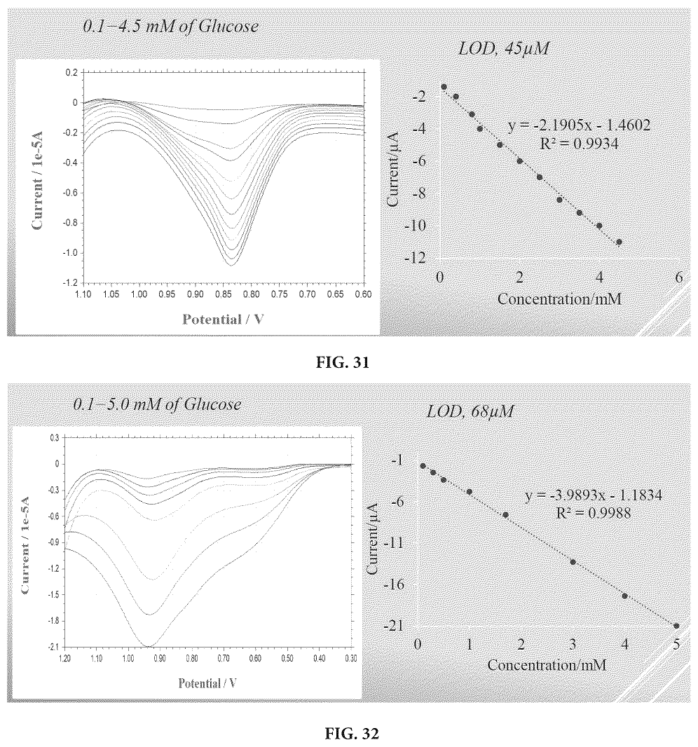

The electrode was electropolymerized with EDOT (3,4-Ethylenedioxythiophene) for 5 cycles and heated at 100° ° C. for 5 minutes, then two 10 microliters of PPMP, BSA, Glut, GOx were added, on polymerized Au electrode. (FIG. 31)

The electrode was electropolymerized with EDOT for 5 cycles, then two 10 microliters of PPMP, BSA, Glut, GOx were added, on polymerized Au electrode. (FIG. 32)

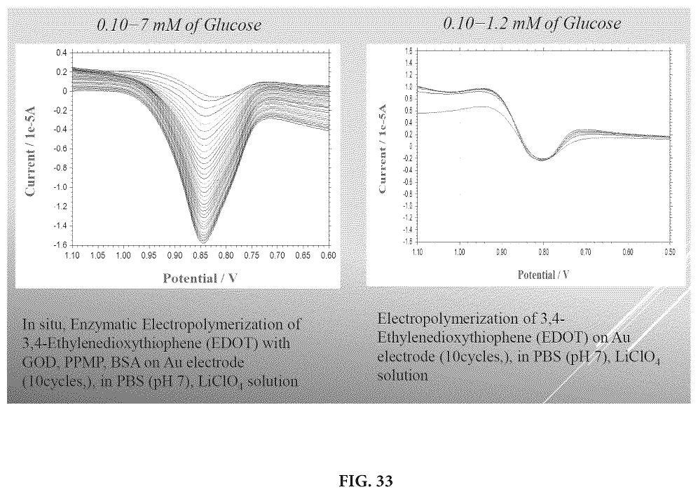

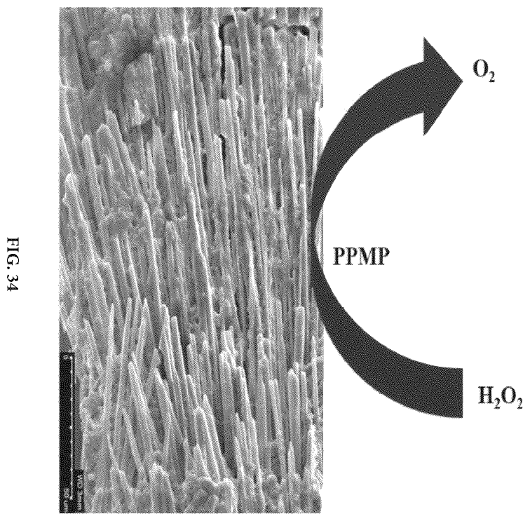

In situ, Enzymatic Electropolymerization of 3,4-Ethylenedioxythiophene (EDOT) with GOx, PPMP, BSA on Au electrode (10 cycles), in PBS (pH 7), LiClO₄ solution (FIG. 33) and Electropolymerization of 3,4-Ethylenedioxythiophene (EDOT) on Au electrode (10 cycles), in PBS (pH 7), LiClO₄ solution

Conclusions
We fabricated biosensors, which is not only cost effective, simple, sensitive, selective, and stable but also able to respond to a wide range of glucose concentrations
To overcome the electron transfer issue between the electrode surface and protein we will use a nanoparticle-based solid electrode
Nanomaterials are utilized improving this electron transfer limitation due to excellent conductivity, high catalytic activity, increasing the number of active sites on the surface and good adsorption of the analyte.

### Example 7—Biosensor for Glucose Determination Based on a Recombinant Mn Peroxidase from Corn Cross-Linked to a Modified Gold Electrode

Using a recombinant enzyme derived from corn and a simple modification, we are fabricating a facile, fast, and cost-beneficial new biosensor to measure glucose. We are applying Plant Produced Mn Peroxidase (PPMP), glucose oxidase (GOx), Bovine serum albumin (BSA), conductive polymer on Au electrode using electrochemical response to detect glucose. The results are compared with glass carbon electrode to show the advantage of each in the use of this enzyme.

The performance and strength of this new enzyme biosensor is simplicity, sensitivities, selectivity, and low limits of detection. Without wishing to be bound by theory, the modified biosensor can be used to monitor various biofluids

### Example 8—Biosensor for Glucose Determination Based on a Recombinant Mn Peroxidase from Corn Cross-Linked to a Modified Gold Electrode

Outline
H₂O₂ sensory using PPMP
Glucose sensor using PPMP
Enhance the sensitivity and stability of the biosensor using nanoparticles and conductive polymers

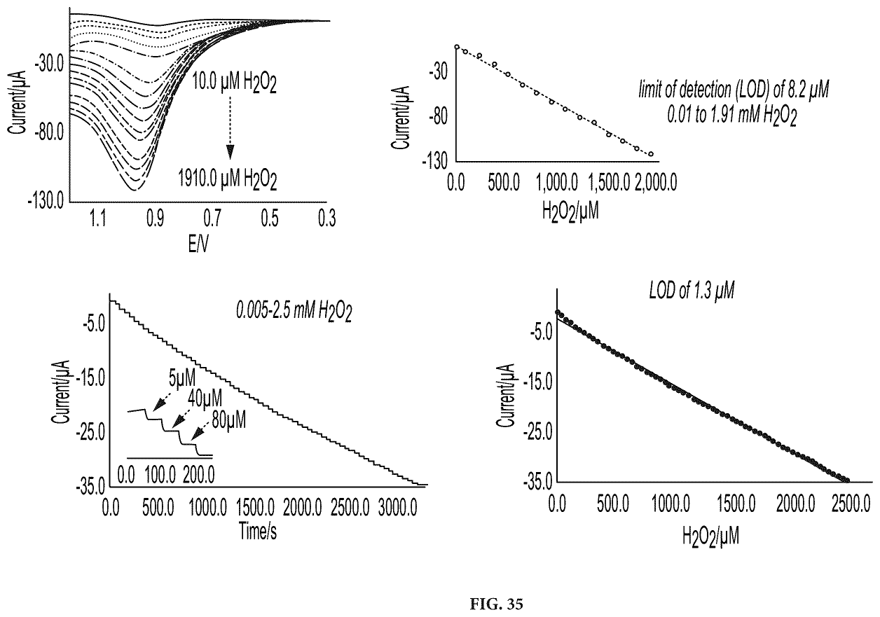

Linear Sweep Voltammetry (LSV), Amperometric Responses of the PPMP/Au electrode to H₂O₂. (FIG. 35)

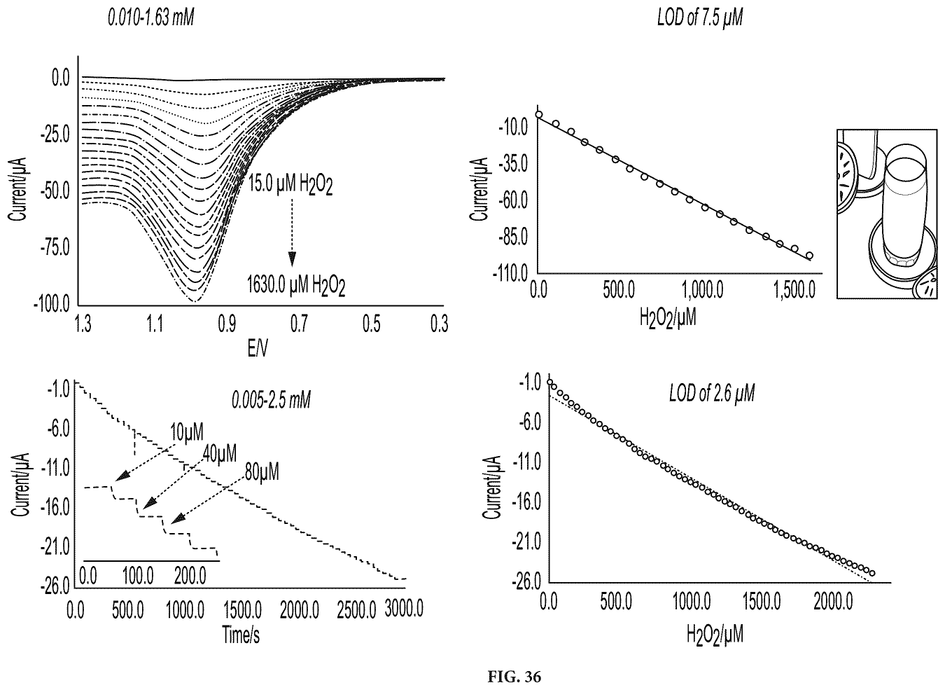

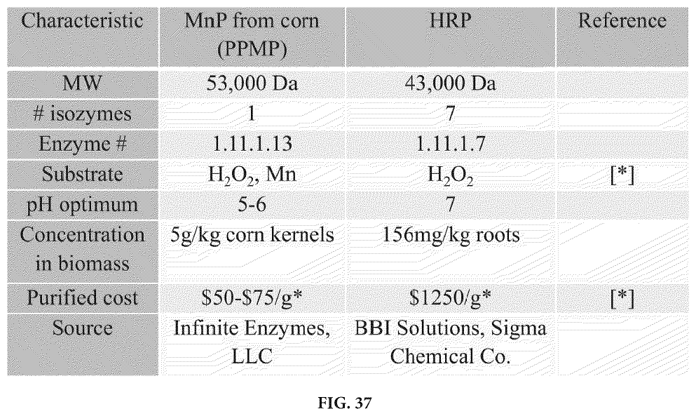

Selective Evaluation of the PPMP Biosensor in Orange Juice (FIG. 36)
GMP at scale, Cost comparison of PPMP and HRP (FIG. 37)

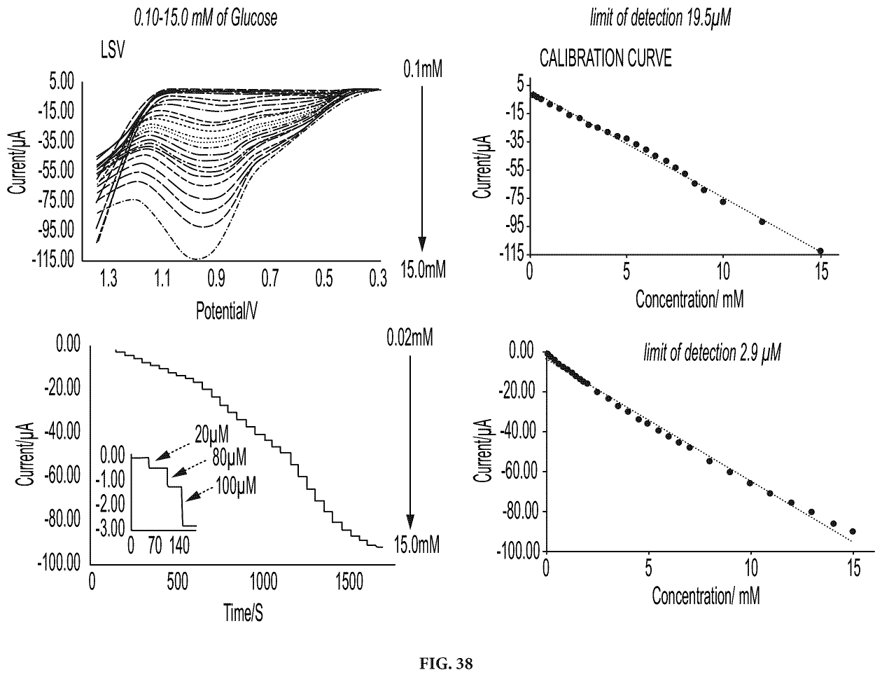

Linear Sweep Voltammetry (LSV), Amperometric Responses of the Nafion™/PPMP-Gox-BSA/Au Electrode to detect glucose (FIG. 38)

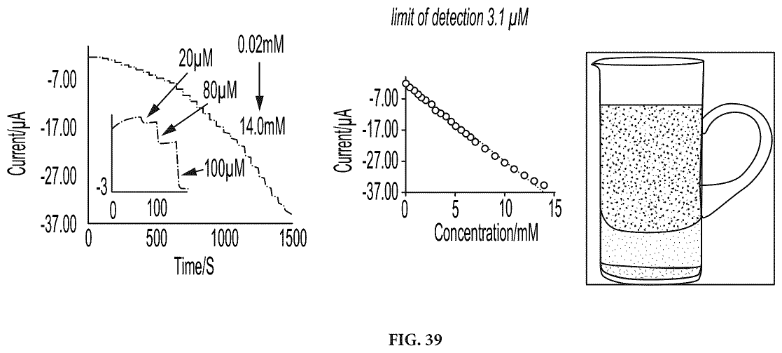

Selectivity of the Nafion™/PPMP-Gox-BSA/Au Electrode (FIG. 39)

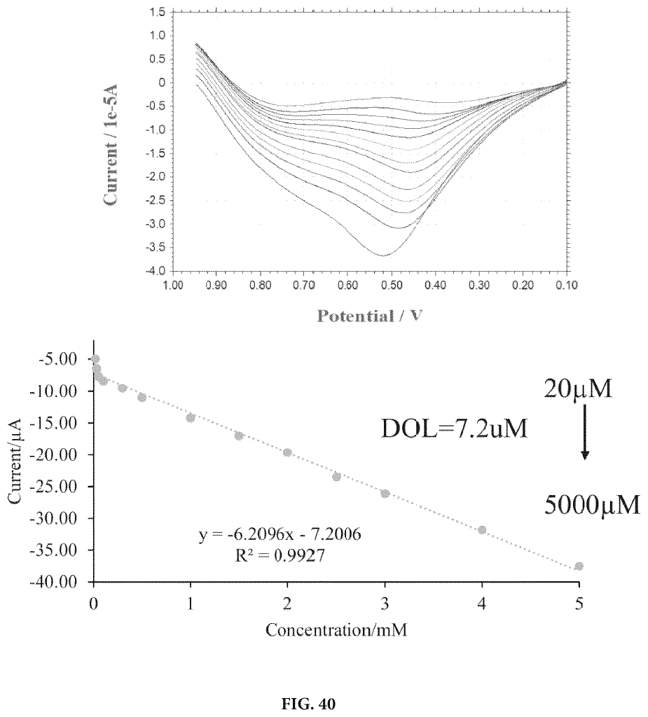

Linear Sweep Voltammetry (LSV), Gold electrode, Electropolymerized with Aniline, GOx, PPMP, BSA (FIG. 40)

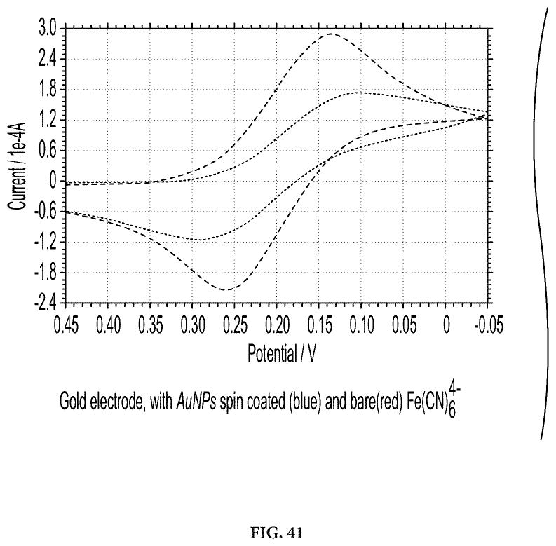

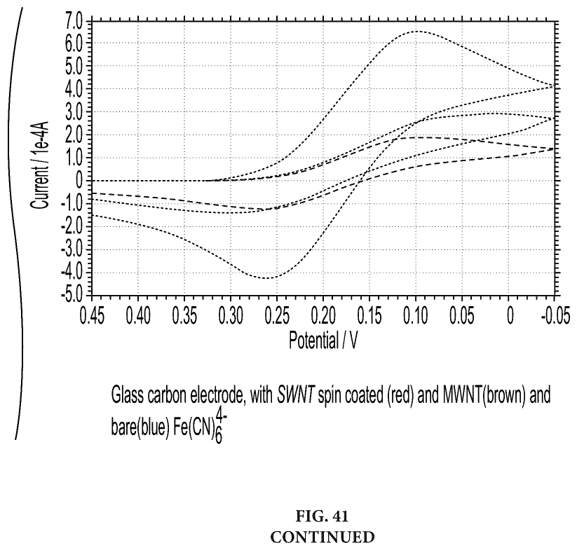

Cyclic Voltammogram with Nanoparticle (FIG. 41)

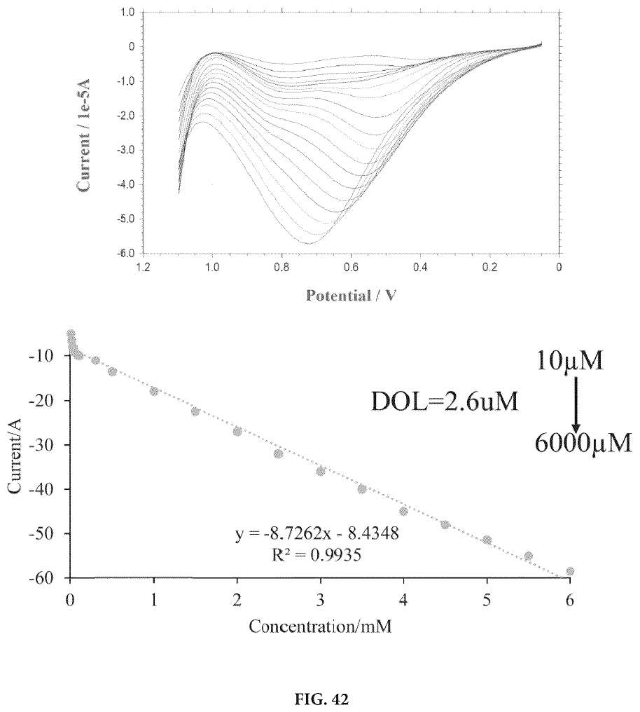

Linear Sweep Voltammetry (LSV), Gold Electrode, Electropolymerized with Au, nanoparticle (AuNPs), Aniline, GOX, PPMP, BSA (FIG. 42)

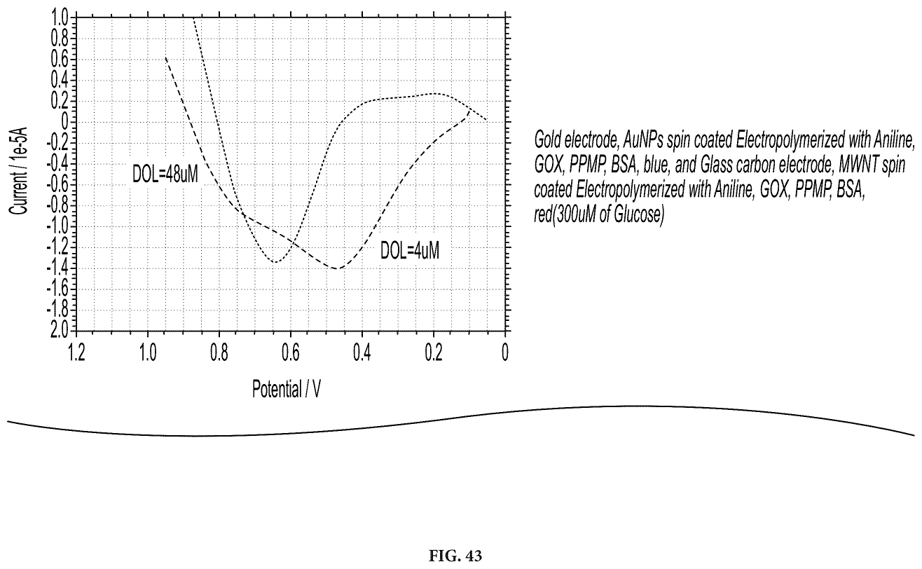

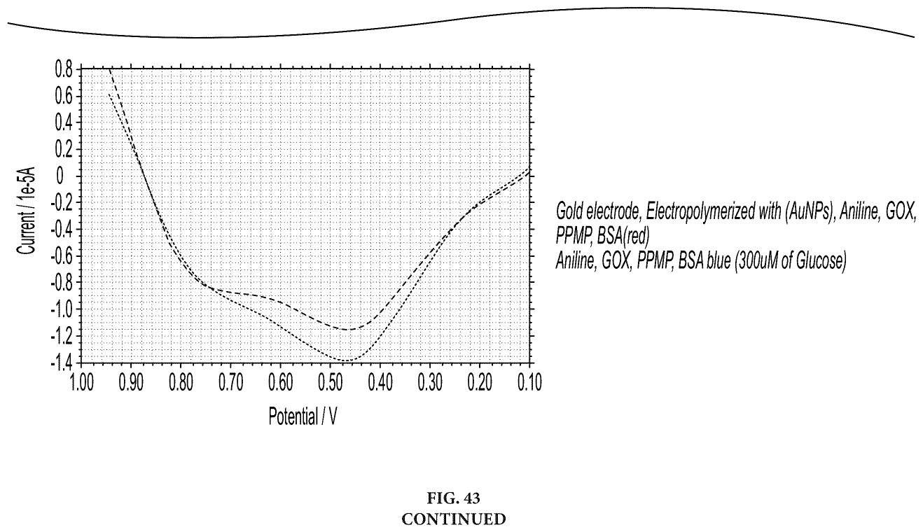

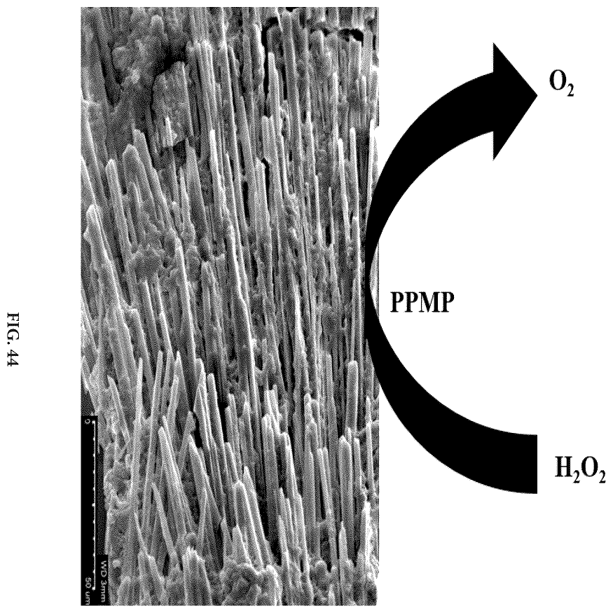

Gold Electrode, AuNPs Spin Coated Electropolymerized with Aniline, GOX, PPMP, BSA, blue, and glass carbon electrode, MWNT spin coated electropolymerized with aniline, GOX, PPMP, BSA, red (300 μM of Glucose) (FIG. 43)

Gold electrode, electropolymerized with (AuNPs), aniline, GOX, PPMP, BSA (red) aniline, GOX, PPMP, BSA blue (300 μM of Glucose) (FIG. 43)

### Example 9—Biosensor for Glucose Determination Based on a Recombinant Mn Peroxidase (PPMP) from Corn Cross-Linked to a Modified Gold Electrode

Outline

H₂O₂—Sensor using PPMP (FIG. 44)

Glucose Sensor using PPMP

Conductive Polymer Nanocomposite Based Biosensors

*GMP at scale

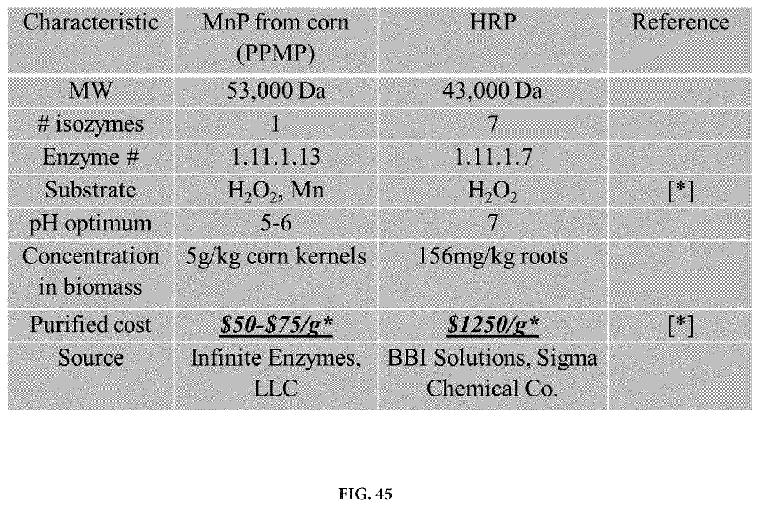

Cost comparison of PPMP and HRP. (FIG. 45)

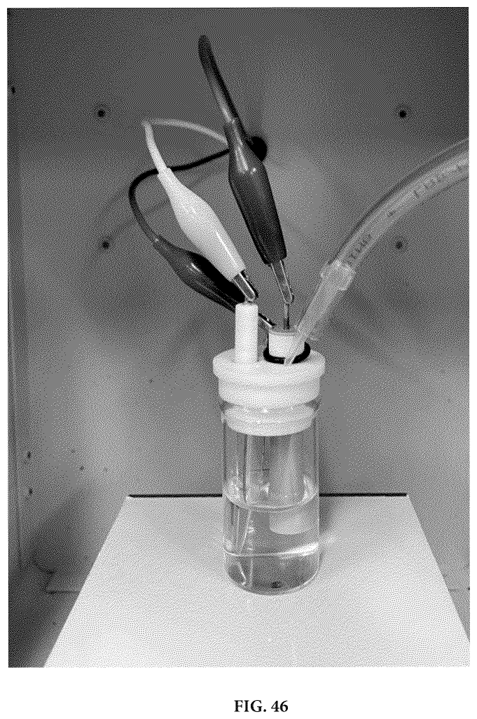

Electrochemical methods, three-electrode cell (FIG. 46)
platinum wire as a counter electrode:
−5 mm-diameter gold electrode as the working electrode:
Ag/AgCl(3 M KCl) as the reference electrode;
0.1 M phosphate buffer (pH 7.0), and 0.1 mM (Mn CH₃COO)₂ solution:

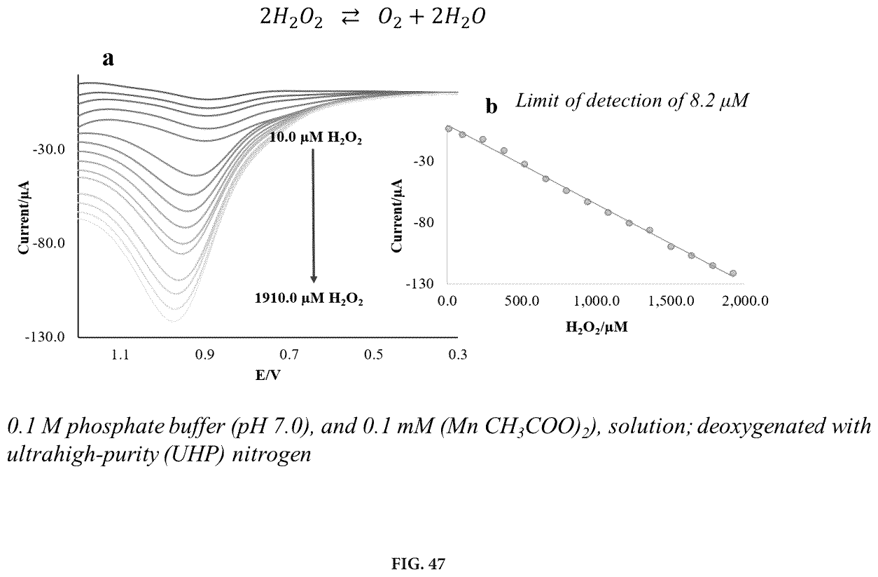

Linear Sweep Voltammetry (LSV), Responses of the PPMP(Na-tartrate buffer pH 4.5)/Au electrode to H₂O₂. (FIG. 47) 0.1 M phosphate buffer (pH 7.0), and 0.1 mM (Mn CH₃COO)₂), solution; deoxygenated with ultrahigh-purity (UHP) nitrogen

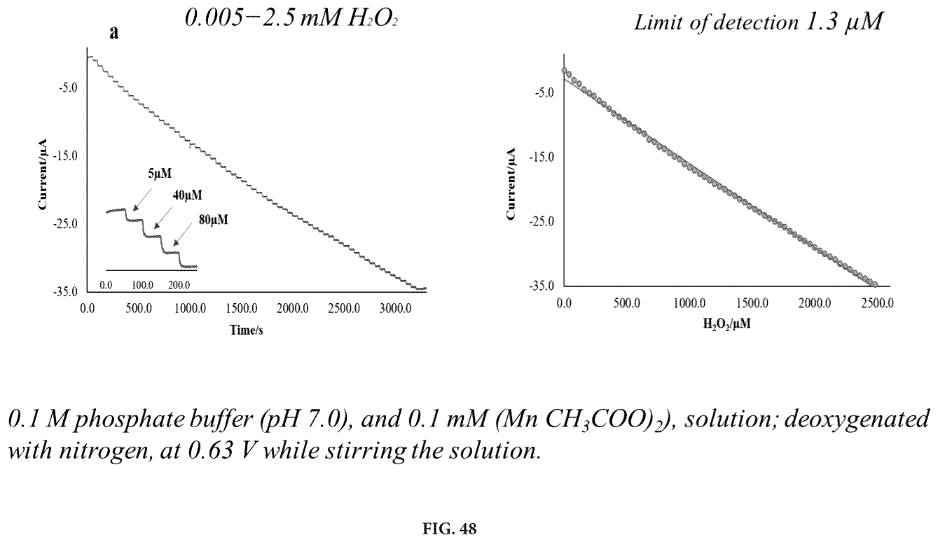

Amperometric Responses of the PPMP(Na-tartrate buffer pH 4.5)/Au electrode to H₂O₂. (FIG. 48) 0.1 M phosphate buffer (pH 7.0), and 0.1 mM (Mn CH₃COO)₂), solution; deoxygenated with nitrogen, at 0.63 V while stirring the solution.

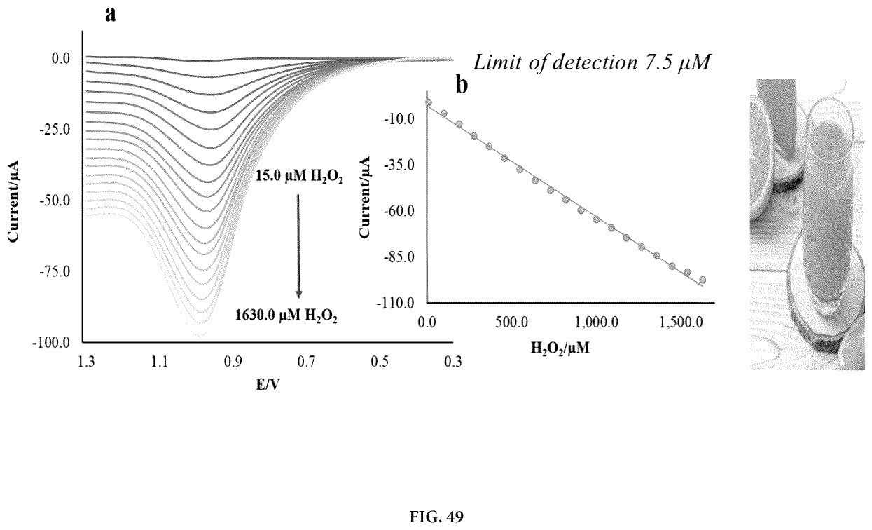

Linear Sweep Voltammetry (LSV), Selectivity Evaluation of the PPMP Biosensor in Orange Juice. (FIG. 49) Orange juice contains ascorbic acid (vitamin C) and other vitamins and citric acid, an organic acid, protein, dietary fiber, sugars, and minerals, calcium, iron, magnesium, phosphorus, potassium, sodium, and zinc.

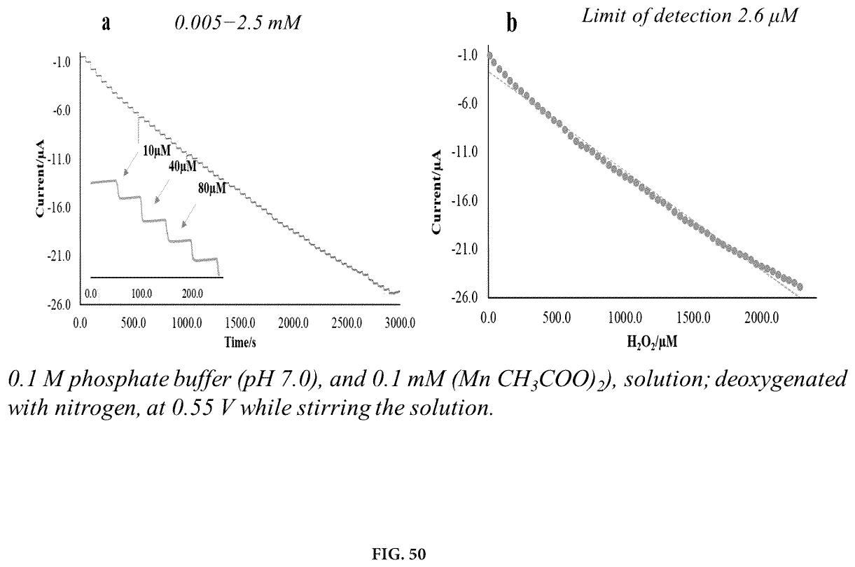

Amperometric, Selectivity Evaluation of the PPMP Biosensor in Orange Juice. (FIG. 50) 0.1 M phosphate buffer (pH 7.0), and 0.1 mM (Mn CH₃COO)₂), solution; deoxygenated with nitrogen, at 0.55 V while stirring the solution.

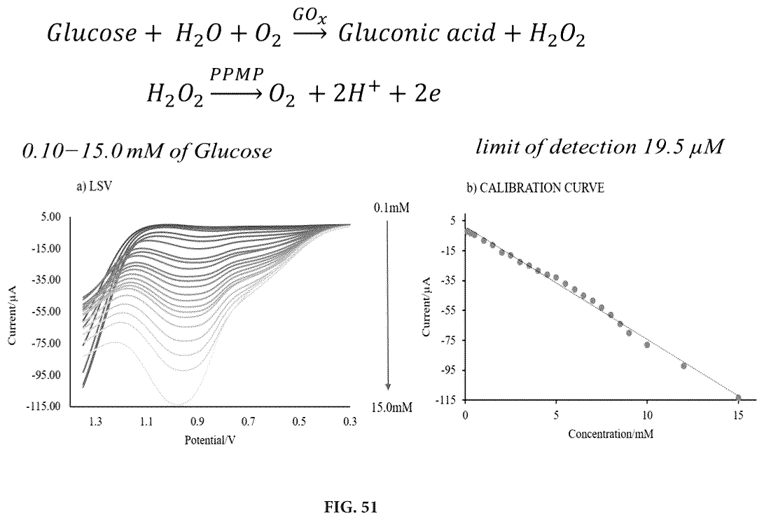

Linear Sweep Voltammetry (LSV), Responses of the Nafion™/PPMP-GOx-BSA/Au Electrode to detect glucose (FIG. 51) Spin coating of 15 μL of enzymes composite; followed by 7 μL of the 0.05% Nafion™; PBS (pH 7.0)/0.1 mM Mn (CH₃COO)₂; saturated with oxygen.

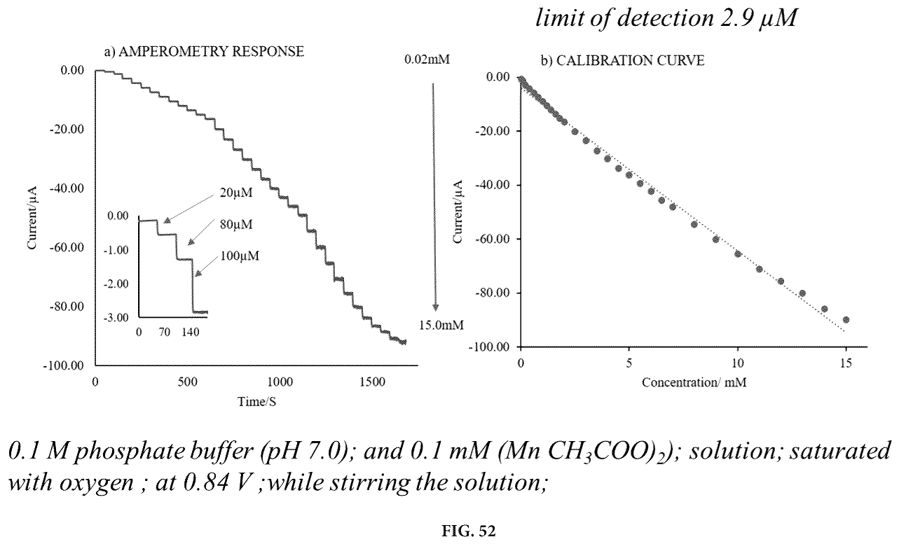

Amperometric, Responses of the Nafion™/PPMP-GOx-BSA/Au Electrode to detect glucose (FIG. 52). 0.1 M phosphate buffer (pH 7.0); and 0.1 mM (Mn CH₃COO)₂); solution; saturated with oxygen; at 0.84 V; while stirring the solution;

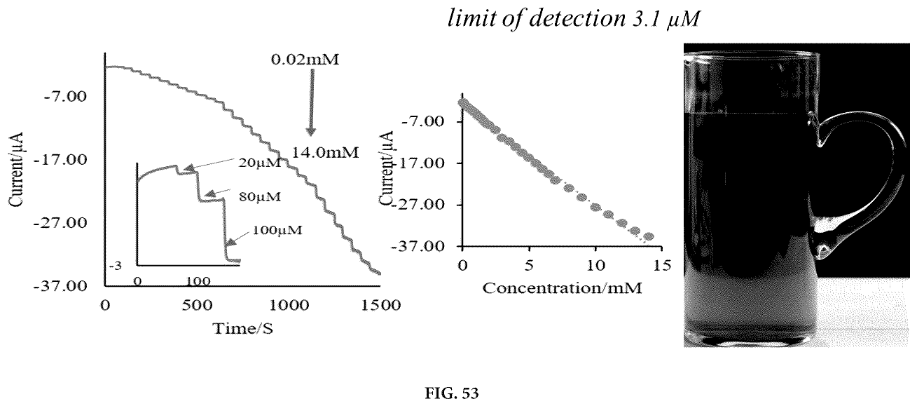

Amperometric, Selectivity of the Nafion™/PPMP-GOx-BSA/Au Electrode. (FIG. 53) 10% diet green tea; contains green tea; citric acid; sodium hexametaphosphate; natural flavor; ascorbic acid (vitamin C); phosphoric acid; potassium sorbate; aspartame; . . . , at 0.84 V; while stirring the solution.

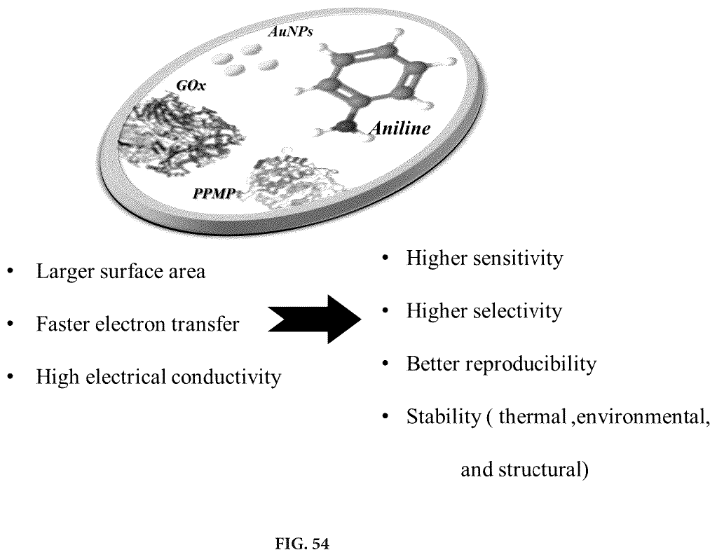

Conductive Polymer Nanocomposite Based Biosensors (FIG. 54)

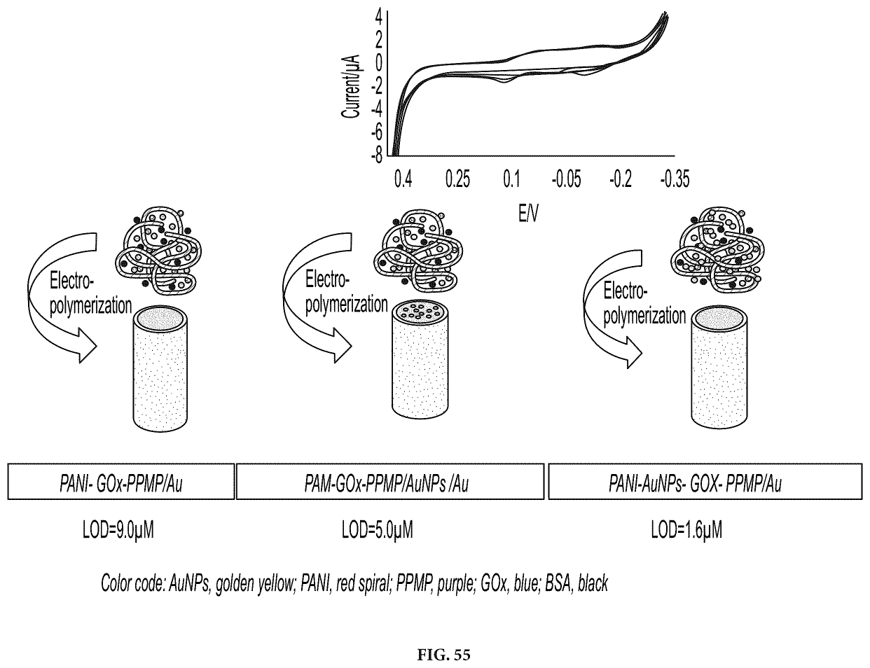

Electropolymerization (FIG. 55)

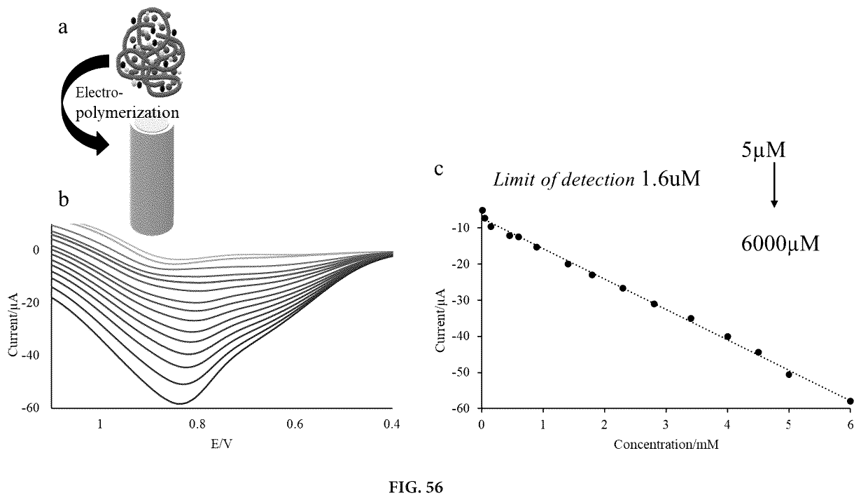

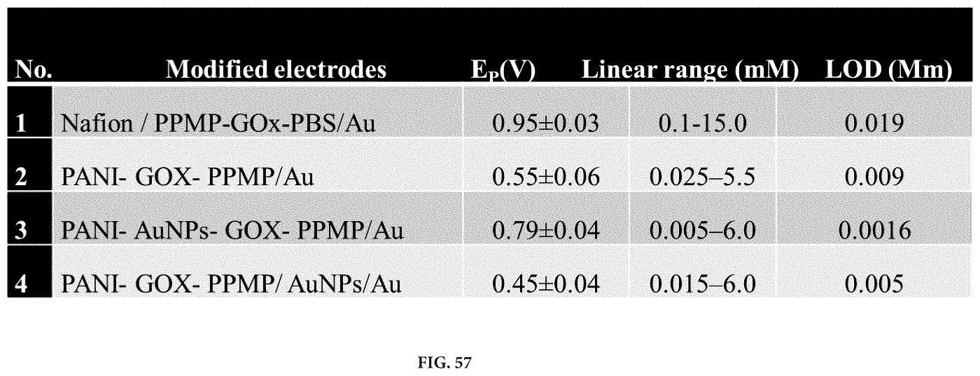

Linear Sweep Voltammetry (LSV), Gold electrode, Electropolymerized with Au, nanoparticle (AuNPs), Aniline, GOx, PPMP, BSA (FIG. 56) Gold electrode, Electropolymerized with 15 cycles of Au, nanoparticle, Aniline, GOX, PPMP, BSA in PBS (pH 7.0).

Comparison LSV of Glucose Sensor Data Using PPMP (FIG. 57)

### Example 10—Bienzymatic Nanocomposite Biosensors to Measure Glucose

Background: Biosensors have attracted due to rapid and real-time health care monitoring, high specificity, and low quantity utilization of sample. Clinical trials and research efforts have shown that monitoring has a high potential for providing early signs of various disorders and diseases. Plants can be a valuable and cost-effective source for producing well-structured recombinant enzymes. Glucose metabolism occasionally becomes uncontrolled causing a myriad of health problems. Worldwide, the number of people with diabetes is currently estimated by the International Diabetes Federation at around 500 million, and this number is steadily increasing. Electrochemical sensor systems can easily be miniaturized and move to nanotechnology. Enzyme-based electrochemical bioassays are robust, selective, sensitive with excellent detection limits and ability to be used for real-time monitoring of complex sample.

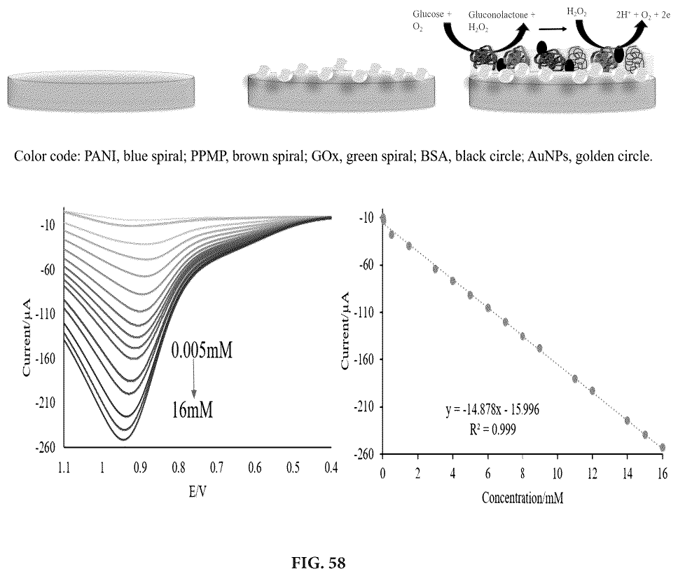

Results: A glucose biosensor was constructed using polyaniline (PANI) and a recombinant enzyme from corn, Plant-Produced Manganese Peroxidase (PPMP), with polymerization of aniline as a monomer in the presence of gold nanoparticles (AuNPs)—glucose oxidase (GOx), and bovine serum albumin (BSA). Using electrochemical techniques, fabricated gold biosensor exhibited a superior sensing property with a wider linear range of 0.005 to 16.0 mM, and a lower detection limit (LOD) of 0.001 mM. The biosensor selectivity was assessed by determining glucose concentrations in the presence of ascorbic acid (AA), dopamine (DA), Aspartame, and Caffeine. See FIG. 58.

Outcome: a plant-produced Mn peroxidase enzyme combined with CPs and AuNPs results in a nanocomposite biosensor to detect glucose. The use of such devices for quality control in the food industry and diabetic monitoring can have a significant economic impact.

### REFERENCES CITED IN THIS EXAMPLE

Izadyar A., Rodriguez K. A., Van, M. N., Tran U., and Hood E. E. “A Journal of Electroanalytical Chemistry. 2021, 895, 115387
Izadyar A., Tran U., and Hood E. E. ACS Sustainable Chem. Eng. 2019, 7, 19434-19441.

### Example 11: Electrochemical Glucose Biosensor

Glucose is one of the most important biological molecules. Worldwide, the number of people with diabetes is currently estimated by the International Diabetes Federation at around 500 million, Glucose monitoring can be expensive and complicated.

Our biosensor is cost effective and innovative as it is based on a corn derived enzyme.

We want to help economically disadvantaged individuals to have access to technologies that can allow them to manage their illness.

New Recombinant Manganese Oxidase Enzyme

Uses Oxygen to Reduce Glucose to H₂O₂

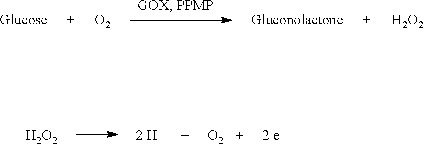

Improvement of the Technology:

Achieve a Lower Detection Limit by Implementing New Technologies

The electrode exhibited superior sensing property with a wide linear range of 0.005 to 16.0 mM, and a low detection limit (LOD) of 0.001 Mm.

How we are Improving the Biosensor:

By coimmobilized recombinant enzyme from corn, Plant-Produced Manganese Peroxidase (PPMP), gold nanoparticles (AuNPs), and glucose oxidase (GOx), on a gold electrode by electropolymerization of, polyaniline (PANI) to demonstrate a stable, sensitive, selective, low-cost, and flexible glucose sensor.

Electrochemistry

The sensing ability of the thin films with respect to glucose was evaluated by Linear sweep voltammetry (LSV) which shows a good controllability and reproducibility of this glucose sensor.

Laboratory Techniques

A mixer was provided including PPMP, Aniline, GOx, in PBS (pH 7.0) and then, the Au electrode was immersed into this composite.

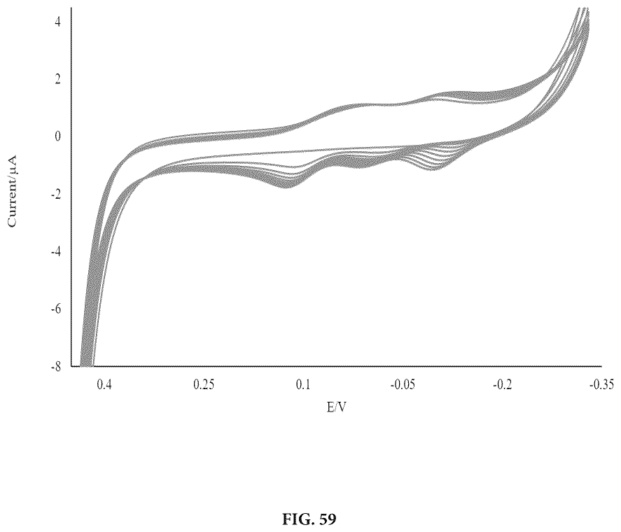

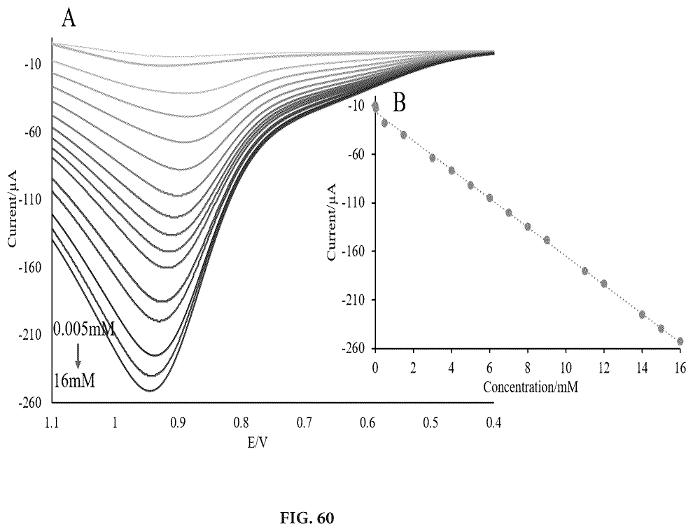

Polymerizations were conducted by cyclic voltammetric sweep of 20 cycles with potential ranging from −0.25 to 0.45 V/Ag/AgCl, at a scan rate of 0.05 Vs⁻¹. (FIG. 59)

Results

Panel (A) Background-subtracted of LSVs for a PANI-GOx-PPMP-AuNPs/Au electrode, in the oxygen saturated PBS/0.1 mM Mn(CH₃COO)₂ solution in the range of 0.005 to 16.0 mM of glucose vs Ag/AgCl (Scan rate of 0.05 Vs⁻¹). Panel (B) Calibration plot of background-subtracted peak current versus glucose concentration (R²=0.9990). (FIG. 60)

Outcomes:

The combined matrix of PANI, AuNPs, GOx, and PPMP, allowed lower detection limits.

This system possesses higher sensitivity and selectivity towards glucose oxidation.

The sensor is highly reproducible, cost effective, and stable.

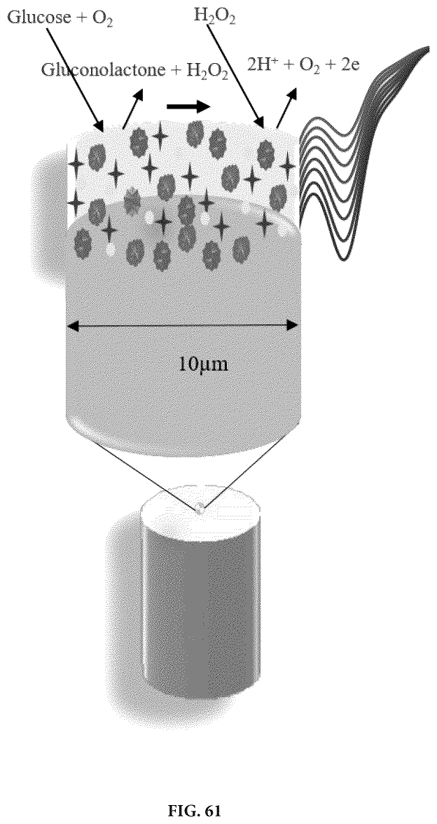

### Example 12—Dual-Enzyme Amperometric Microbiosensors Using Polyaniline Co-Immobilized with Recombinant Enzyme from Corn, Glucose Oxidase, and Gold Nanoparticles to Detect Glucose (FIG. 61)

Abstract

Our report revealed that recombinant enzyme from corn are effective and suitable for glucose monitoring. However, we extended our work by modification of a 10 μm (10×10⁻⁶ m) diameter gold microelectrode, (GME) which guide this biosensor towards practical applications and in vivo analytical monitoring of glucose. The polyaniline (PANI)—gold nanoparticles (GNPs)—glucose oxidase (GOX)—plant-produced manganese peroxidase from corn (PPMP)/gold microelectrode (GME) composition showed excellent chronoamperometric (CA) response for the selective and sensitive enzymatic detection of glucose. PANI-GNPs-GOX-PPMP/GME applying CA exhibits a wide linear range of 0.001 to 16.0 mM (10-3 molL⁻¹), and a lower detection limit (LOD) of 0.50 μM (0.5×10⁻⁶ molL⁻¹). Linear sweep voltammetry (LSV) and cyclic voltammetry (CV) were also applied for further electrochemical characterization of modified microelectrode to detect glucose. The biosensor selectivity was validated by determining glucose concentrations in the presence of ascorbic acid (AA), dopamine (DA), aspartame, fructose, uric acid (UA) and caffeine. As a result, the new dual enzyme microbiosensors can facilitate medical diagnosis and monitoring glucose levels of foods.

### Introduction

In recent decades, electrochemical biosensors have received tremendous attention and is mainly focused on the healthcare industry. Clinical trials and research efforts have shown that monitoring has a high potential for providing early signs of various disorders and diseases. With this in mind, the development of biosensors to glucose monitoring has become the subject of intense scientific for various research groups around the world. [1,2] Enzymatic biosensors are used to monitor the concentration of molecules in vivo due to faster and cost-effective diagnostic devices. Enzyme-based biosensors have the largest commercial biosensors market and are widely studied. The selectivity of enzymatic biosensors influenced by design parameters and materials used to create the biosensors, modifiers, and enzymatic stabilization methods to the performance of biosensors.[3]

Today, we enjoy the results of nanotechnology for a hassle-free life. Nanotechnology also works with the help of sensors and biosensors.[4] With advances in biosensors, studies are underway to make them smaller and more portable due to various applications, such as medical diagnostics, environmental monitoring, healthcare and industrial production [5,6], increase spatial resolution, selectivity, in vivo imaging [7] and chemical sensors. [8] Microscale enzyme electrodes that work in small volumes, has attracted more attention due to increasing the signal-to-noise ratio and reduce the sample size. To create better miniature biosensors, a large quantity enzyme can be immobilized on the microelectrode. Nanomaterials can be used as a solid support to collect more enzymes on the electrode surface due to its large surface area. Additionally, nanomaterials have unique advantages of direct and fast electron transfer between enzyme active sites and the electrode, [9, 10] which reduce of the response time and improve accuracy. Gold nanoparticles (GNPs) play important roles in biosensors. [11-14] These nanoparticles can be used for biomedical studies due to their unique properties.

Electrochemical biosensors are very powerful electroanalytical technique that has the advantages of high sensitivity, simplicity, portability, easy downsizing and low cost. Researchers have made great efforts to improve these properties, and one of the ways is to include nanomaterials in biosensors. Due to the importance of glucose detection for patients with diabetes, many glucose sensors have been developed in the past 3 decades. However, there are still several challenges associated with developing a small device that can quickly, and reliably monitor glucose in vivo. Among few different techniques, electrochemical methods are techniques for continuous sugar monitoring. [16, 17] Amperometric biosensors are integrated devices where the biochemical receptor is maintained in direct contact with the electrochemical transmission element. Enzyme-based electrochemical bioassays are robust, selective, sensitive with excellent detection limits and ability to be used for real-time monitoring of complex sample. [11,12]

Flexible sensors with incorporated conductive polymers can be used in different fields. Conductive polymers (CPs) can improve biosensors by increasing the effective surface area, reducing the impedance, and improving the bioelectronics signal. CPs can be obtained by electrochemical oxidation of aromatic monomers on the electrode surface to make a homogeneous thin film. [19-26] Conductive polymer (CP) nanomaterials have significant physical and chemical properties for cost-effective, large-scale, lightweight, flexible biosensors, and improving in vivo usage due to antifouling biosensing electrochemical interfaces. Nanobiosensors based on PANI nanomaterials have shown high performance sensory properties toward biological targets and exhibit excellent properties such as long-lasting, and non-toxicity. [27-31]

Horseradish peroxidase (HRP) with GOx can be used for glucose detection. However, HRP has several disadvantages including high cost and finite reaction conditions. In our pervious works. [17, 32] we used plant-produced manganese peroxidase from corn (PPMP) to produce a successful cost-effective dual enzyme biosensor. The enzyme gene (Genbank access #: 129039) was cloned from the white rot fungus Phanerochaete chrysosporium and expressed recombinantly in corn kernels.

In this study, we take advantage of PPMP, GOx, GNPs, and PANI to construct a new microbiosensor to measure glucose using electrochemical technique. We indicate one step PANI electropolymerization procedure using PPMP, GOx and GNPs on a 10 μm of a GME. A purpose of this study was to further validate analytical applications of plant-produced manganese peroxidase from corn-based biosensors towards developing an economical, rapid, and appropriate method for determining biologically important compound, glucose. Under the optimum operational conditions, the PANI-GNPs-GOx-PPMP/GME showed a good sensitivity, selectivity, rapid response time and excellent operational stability to monitor glucose. Further, this unique fabricated micro scale electrode will lead to the use of this electrochemical microbiosensor for in vivo application.

Materials and Methods

Chemicals

PPMP was prepared as before. Manganese (II) acetate (Mn(CH₃COO)₂), BSA, GOx from Aspergillus niger, Type X-S. Phosphate buffer solution (PBS) using NaH₂PO₄, and Na₂HPO₄, GNPs (ultra uniform, 10 nm. 0.05 mg/mL (aqueous 2 mM sodium citrate)), aniline, AA, DA, aspartame, fructose, UA and caffeine were purchased from Sigma-Aldrich (St. Louis, MO). Solutions were prepared in Milli-Q deionized water (18.2 MΩ) unless otherwise noted. Experiments were conducted at laboratory room temperature (20° C.).

Apparatus

All voltammetry and amperometric measurements were performed using a computer-controlled CHI660D electrochemical workstation (CH Instruments, Austin, TX). Experiments were carried out in a three-electrode cell with a platinum wire (0.25 mm diameter, 99.9%, Alfa Aesar. Ward Hill, MA) as a counter electrode and a 10.0 μm diameter GME as the working electrode, and Ag/AgCl (3 M KCl) as the reference electrode.

Ag/AgCl∥xM(glucose),0.1 M PBS(pH 7.0),0.1 mM(Mn(CH₃COO)₂)|polymer matrix((PPMP(0.5 M)-aniline(0.17 M)-GOx(0.10 M)-BSA(2.4×10⁻⁶ M)—GNPs(1.0 g))|GME(10 μm)| (cell 1)

Scanning electron microscope (SEM) images were recorded on an SNE-4500 M plus tabletop scanning electron microscope nano images. Benchtop chilling/heating incubators (Fisher Scientific Company).

Electrodeposition Procedure

The electrodeposition modification procedure was optimized to reach the maximum sensitivity. A 10 μm diameter gold microelectrode cleaned using electrochemical method by cyclic voltammetry in 1.0 M H₂SO₄ and it was sweeping with potential between −0.30 to +1.2 V/Ag/AgCl, at a scan rate of 0.05 Vs⁻¹ for 30 cycles, then the microelectrode cleaned with the same conditions in PBS. Electropolymerization depositions on GMEs were performed using 0.50 M PPMP, 0.17 M aniline. 0.10 M GOx, and (2.4×10⁻⁶ M) BSA in PBS (pH 7.0) solution by cycling of 15 to 30 cycles with potential between −0.42 to 0.50 V/Ag/AgCl, at a scan rate of 0.05 Vs⁻¹. Nanocomposite film shows the best stability using 25 cycles used in all of our electrode modifications. The modified GME was called PANI-GNPs-GOX-PPMP/GME and the prepared electrodes were stored in an incubator at 4° C. when not in use.

Results

Electrochemical Sensing of PANI-GNPs-GOx-PPMP/GME.

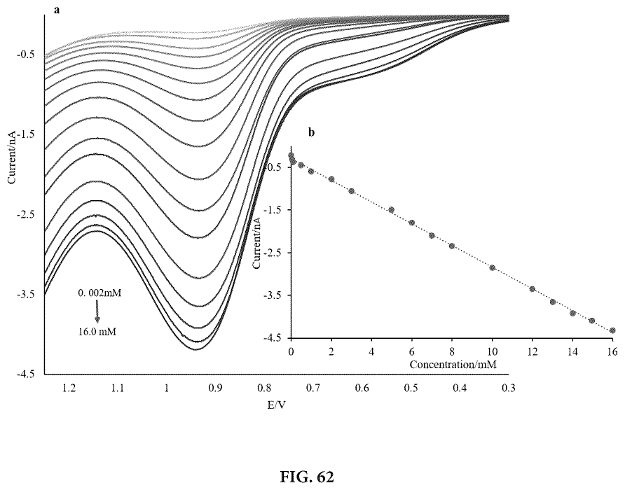

The electro-catalytic activity of PANI-GNPs-GOx-PPMP/GME was validated with the LSV, CV and CA techniques to determine the electrochemical sensing behavior of the modified microelectrode. The quantitative determination of glucose in in a PBS (pH 7.0)/0.1 mM Mn (CH₃COO)₂ solution saturated with oxygen gas was carried out using an LSV technique with the PANI-GNPs-GOx-PPMP/GME. FIG. 62 panel a shows the non-background-subtracted LSVs with the successive addition of glucose in the range of 0.002-16.0 mM indicate sharp increase in current at 0.94V potential at scan rate 0.05 Vs⁻¹. The change in the oxidation peak potential to a higher value, indicating an increase in local oxygen concentration at the surface of the PANI-GOx-GNPs-PPMP/GME electrode. [17, 32] The regression lines (R²=0.9993) values were fitted to the calibration curves (FIG. 62 panel b). The limit of detection (LOD) of 0.6 μM was calculated for S/N=3 and S=(3×std dev)+blank.

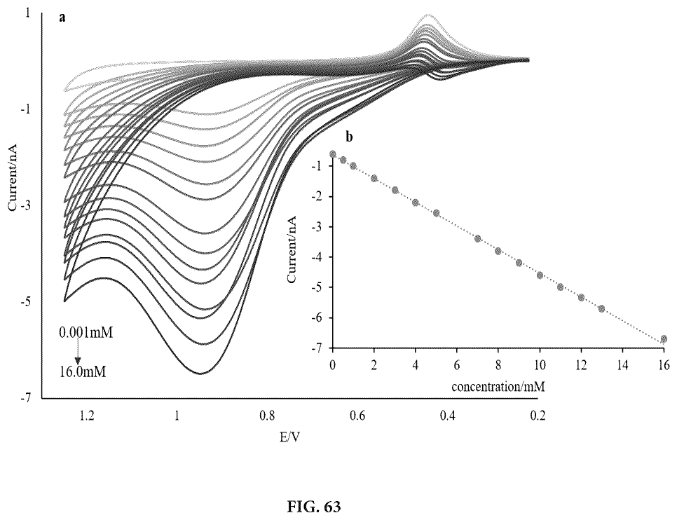

Furthermore, CV as a quick and easy tool to describe glucose biosensors was studies to characterize the electrocatalytic activity of PANI-GOx-AuNPs-PPMP/GME. FIG. 63 panel a shows the microbiosensors CVs with the successive addition of glucose from 0.001-16.0 mM in a PBS (pH 7.0)/0.1 mM Mn (CH₃COO)₂ solution saturated with oxygen gas at scan rate 0.05 Vs⁻¹. As shown in FIG. 63 panel a, the anodic peak current increased linearly at 0.94V on addition of glucose (R²=0.999) with LOD of 0.3 μM (S/N=3)

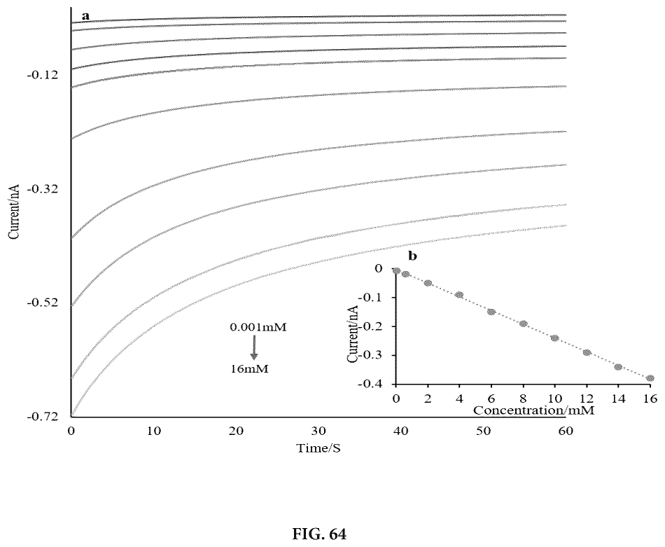

To validate the efficiency of the of PANI-GOx-AuNPs-PPMP/GME as a biosensor, a steady-state CA response was studied. The amperometric results were validated under a static cell in the same conditions described herein. At various concentrations of glucose over the range of 0.001 to 16.0 mM (FIG. 64 panel a) all of the response curves reached steady values quickly within 60 seconds. The maximum current response was observed at 0.75 V while varying the operating potential from 0.60 to 0.90 V. As FIG. 64 panel b shown there was a linear relation between steady-state current and glucose concentration over a wide range with a correlation coefficient of 0.9991 and LOD of 0.5 μM.

Selectivity, Reproducibility and Stability of PANI-GNPs-GOx-PPMP/GME.

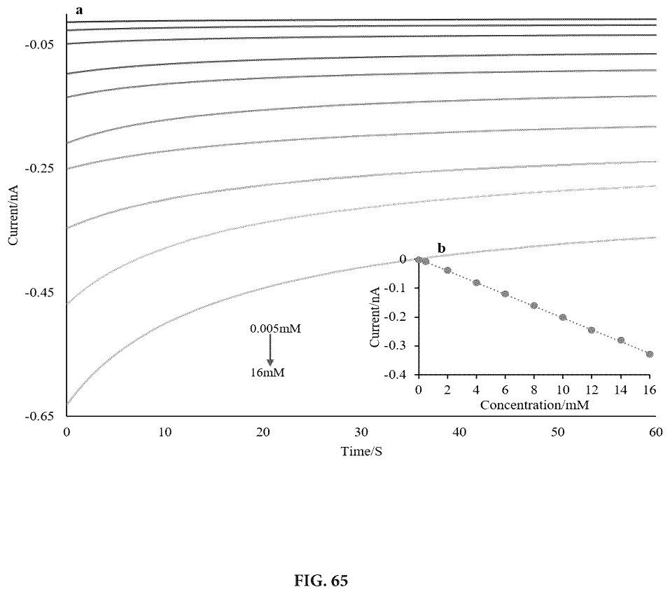

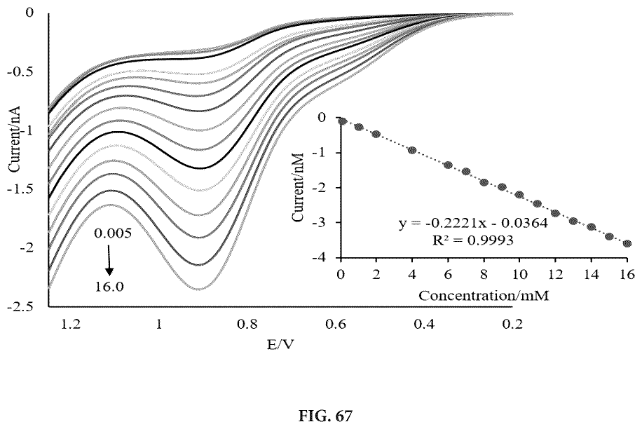

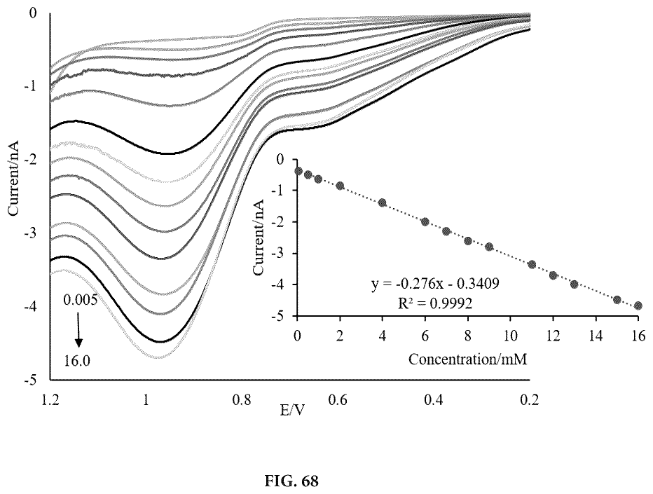

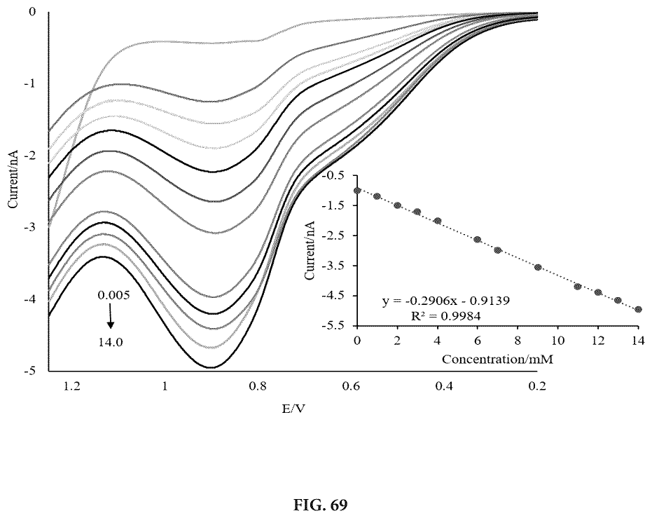

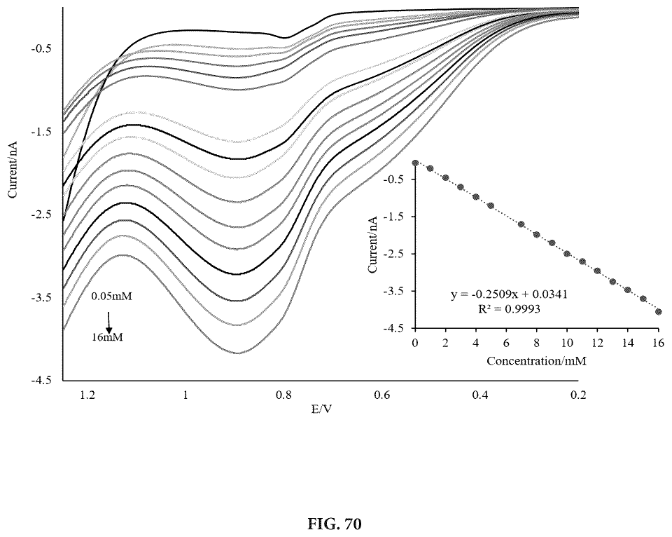

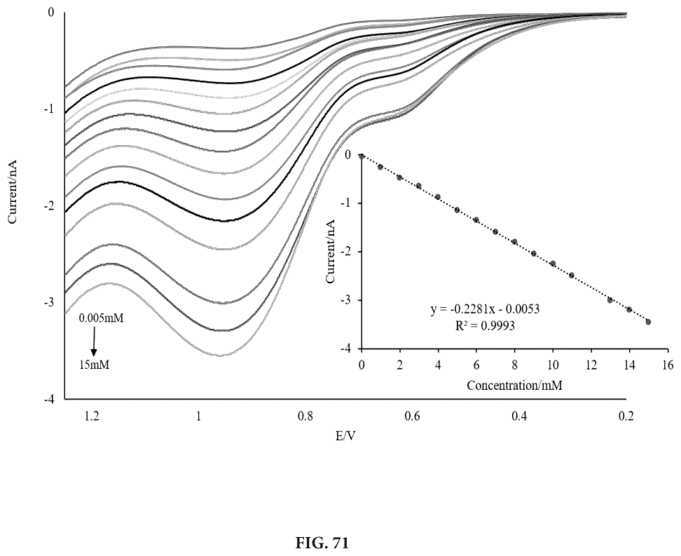

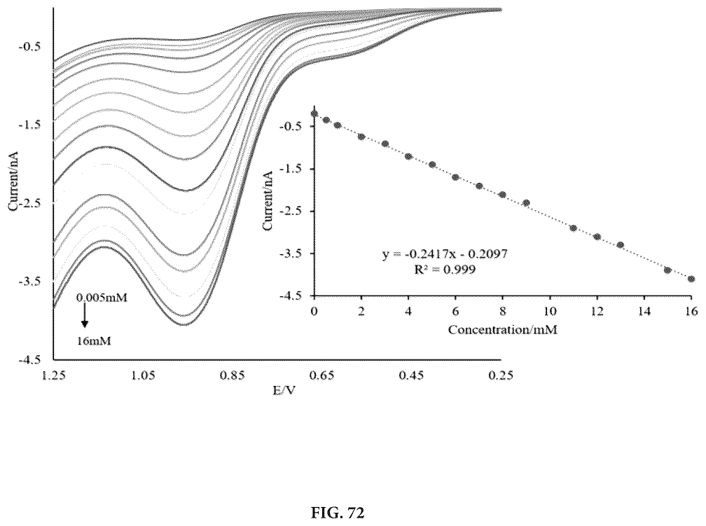

The anti-interference ability of PANI-GNPs-GOx-PPMP/GME validated by LSV technique using AA. DA, aspartame, fructose, UA and caffeine (FIGS. 67-73). Table 4 shows linear range of glucose concentration and LOD in 1 mM constant concentration of interfere compounds. AA was chosen to validate the sensor using CA response on PANI-GNPs-GOx-PPMP/GME under the same cell set up conditions at potential 0.75V. The response curves reached steady after 60 s at various concentrations range of glucose from 0.002 to 16.0 mM at 1.0 mM of AA with LOD of 1.0 μM and R² of 0.9990 (FIG. 65 panel a, b).

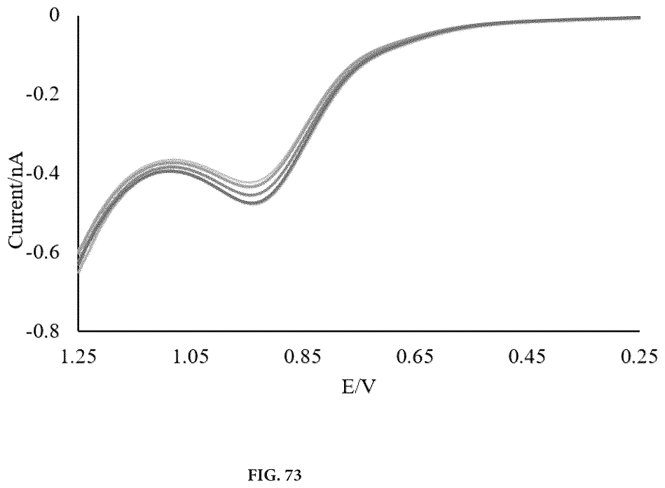

Repeatability of the fabrication of the modified microbiosensor was validated for a total of 7 times consecutive runs in 0.5 mM glucose in a single day using LSV (FIG. 73). The PANI-GNPs-GOx-PPMP/GME were validated for their storage and operational stability by applying LSV and CA techniques. After the as-prepared microelectrode was stored at 4° C. for 2 months the current response only decreased by 5.0% of the original response.

Different Scan Rates Responses of PANI-GNPs-GOx-PPMP/GME.

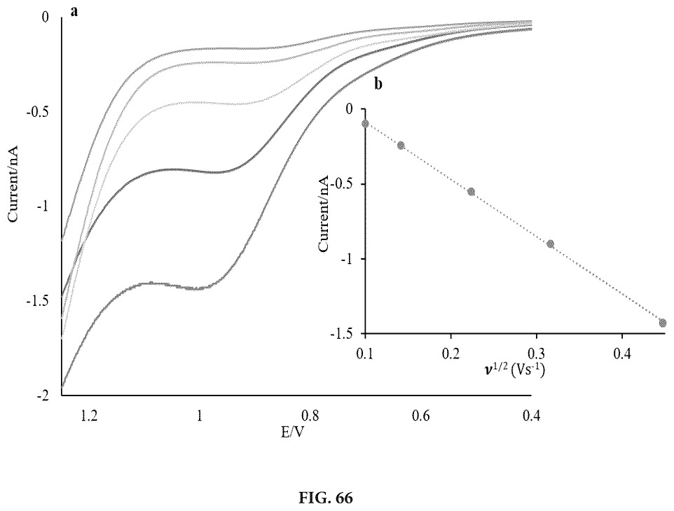

Furthermore, the diffusion-controlled electrochemical behavior of PANI-GNPs-GOx-PPMP/GME was validated by varying the different scan rates from 0.01 to 0.20 Vs 1 in 0.5 mM glucose in PBS pH 7/0.1 mM Mn(CH₃COO)₂ in an oxygen saturated solution (FIG. 66 panel a). The oxidation peaks current increased linearly with the square root of the scan rate (FIG. 66 panel b), indicating a diffusion-controlled process. According to the Randles Sevcik equation:

$$\\ {i}_{p} = ( 2.687 \times {10}^{5} ) {n}^{\frac{3}{2}} A \cdot {C ( D \cdot v )}^{1 / 2}$$

Where peak current (iₚ) depends on the concentration (C), the diffusion coefficient (D), n is the number of electrons transferred, v is the scan rate in Vs⁻¹, and the electrode surface area (A).

Morphology Studies

The surface morphology of the PANI-GNPs-GOx-PPMP/GME compared with the PANI-GNPs-GOx-/GME and the PANI-GOx-PPMP/GME were validated through SEM.

### DISCUSSION

FIG. 62 panel a indicates the results of non-background-subtracted LSVs using PANI-GOx-GNPs-PPMP/GME with the successive addition of glucose. FIG. 62 panel b shows the current response plot against the glucose concentration in a wide range of 0.002-16.0 mM and LOD of 0.6 μM.

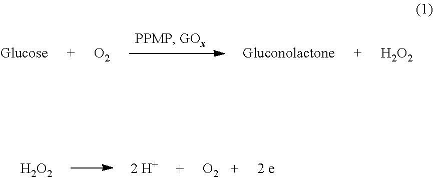

Glucose using PANI-GOx-GNPs-PPMP/GME is quantified by the electrochemical measurement of hydrogen peroxide. Peak currents are based on consumption of oxygen by the enzyme-catalyzed reaction. Oxygen acts as the oxidizing agent to produce H₂O₂ and with oxidation of H₂O₂ at the modified microbiosensor two electrons are transferred directly to the electrode. From FIG. 62 panel b, the current increased sharply with adding glucose. This result demonstrates an excellent electrocatalytic performance of PANI-GOx-AuNPs-PPMP/GME which is due to catalytic activity of PPMP enzyme. The high surface area of GNPs allows immobilization of a large number of enzymes which significantly increasing the sensitivity of the sensor, and a superior electron transfer capability.

In addition, CV was studied to validate the electrochemical sensing behavior of PANI-GOx-AuNPs-PPMP/GME between 0.2 and 1.25 V. As shown in FIG. 63 panel a, the oxidation peak is found to be at 0.94V and the reduction peak at 0.44 V. After sequentially adding glucose to PBS, a significant increasing anodic peak appears at about 0.94 V, accompanied by a decrease of the reduction peak, possesses that shows a high catalytic activity of PPMP enzyme on microelectrode toward the oxidation of the glucose. Indeed, the microbiosensors indicate activity of the biosensor in the wide range from 0.001-16.0 mM of glucose with LOD 0.3 μM (FIG. 63 panel b).

Furthermore, CA measurement was carried out using the PANI-GNPs-GOx-PPMP/GME under a similar cell setup. CA due to hydrodynamic conditions provided a more direct way to characterize sensitivity and selectivity compared to LSV and CV. The amperometric responses of the PANI-GNPs-GOx-PPMP/GME biosensor were validated at different applied potentials of 0.60 to 0.90 V and at an upon the onset of the potential at 0.75 V, the response time was faster compared to other potentials. The microelectrode over the range of 0.001 to 16.0 mM (FIG. 64 panel a) shows excellent sensitivity with LOD of 0.5 μM (FIG. 64 panel b). This result demonstrates an excellent electrocatalytic performance of the PANI-GNPs-GOx-PPMP/GME. LOD comparably is lower than GOx/Au/MXene/Nafion/GCE, GOD/poly-PDA, Nanodiamond-g-PANI/GOx, PANI/MWCNTs/GON/HRP, and Au-Cys-GA-GOx. In addition, the PANI-GNPs-GOx-PPMP/GME has advantages such as quick preparation, being simpler and lower cost.

Selectivity is the most important feature of a sensor since a best selectivity ensures excellent accuracy. The results shown no obviously different responses were observed in comparison with a similar cell setup without Interfering materials. Materials were studied for any interfering effects in glucose analysis. Known concentrations of AA, DA, aspartame, fructose, UA, and caffeine which are electroactive interfering substances for glucose biosensors was added. No significant interfering effects observed at 1 mM of these compounds on the analysis of glucose using CA and LSV methods. LSVs results are summarized in Table 4 and shown in (FIGS. 67-73). The experiments indicated only a minor difference in the responses and these results showed selectivity of PANI-GNPs-GOx-PPMP/GME, therefore, this preparation method for a glucose biosensor can be used for selective monitoring of glucose concentrations. Also, to further validate the microelectrode selectivity, CA applied in the range of 0.002 to 16.0 mM of glucose concentration at constant amount of 1.0 mM of AA (FIG. 65 panel a, 65 panel b) and the LOD was 1.0 μM. In CA experiments, the oxidation current with successive addition of glucose at the potential of 0.73V after 60 s reached a plateau, indicating a quick response to changes in the glucose concentration indicating fast response time and no effect of interfering compound. These results indicate that the PANI-GNPs-GOx-PPMP/GME can also undergo the catalytic oxidation of glucose in the presence of interfering chemicals.

TABLE 4
Influence of electroactive interferents on the
response of the microbiosensor using LSV method

| | Adding | Concentration | |
|---|---|---|---|
| Interferent | content (mM) | range (mM) | LOD(μM) |
| Ascorbic acid | 1.0 mM | 0.005-16.0 | 1.5 |
| Caffeine | 1.0 mM | 0.005-16.0 | 1.7 |
| Dopamine | 1.0 mM | 0.005-14.0 | 2.0 |
| Fructose | 1.0 mM | 0.005-16.0 | 1.4 |
| Uric acid | 1.0 mM | 0.005-15.0 | 1.6 |
| Aspartame | 1.0 mM | 0.005-16.0 | 1.5 |

Additionally, the repeatability of PANI-GNPs-GOx-PPMP/GME was validated, and it shows excellent repeatability with the relative standard deviations (RSD) of value of 4.37% (FIG. 73). Also, the current response was found to remain stable for 2 months. It retained 95% of the initial current response. The results indicate the long-term stability of the electrode, and no disturbance can cause drift in the output signals of biosensor response. Thus, PPMP enzyme is completely stable and can be used for mass production of enzyme biosensors.

Different scan rates responses of PANI-GNPs-GOx-PPMP/GME in the potential windows between 0.2 and 1.25 V were employed to assess the dynamics of heterogeneous electron transfer across the electrode/film layer interface. As the FIG. 66 panel a shows the peak current (iₚ) increases at faster voltage scan rates. This is due to the establishment electrode potential against diffusing of glucose concentration gradient. By increasing voltage, the concentration of glucose at the microelectrode surface also alters, so, a faster sweep of voltage causes a greater concentration gradient near the microbiosensor, making a larger peak current. The oxidation peaks current increased linearly with the square root of the scan rate (FIG. 66 panel b) with R² of 0.9994, indicates that the process was controlled by diffusion and the electron transfer processes involve freely diffusing of glucose.

Outcomes

PANI-GNPs-GOx-PPMP/GME indicates significant reduced of charge transfer resistance while we minimized the size of electrode to 10 μm microelectrode. The results also demonstrated that electrodeposition of GOx-PPMP along with GNPs onto GME is an effective electrochemical technique for improving sensitivity and electron transfer rate to detect glucose. Through electrochemical response using CA, CV, and LSV as analytical techniques, the PANI-GNPs-GOx-PPMP/GME exhibits a wide linear range from 0.005 to 16.0 mM with a detection limit of 0.0026 mM, 0.01 Mm to 16.0 mM with a detection limit of 0.0032 mM and from 0.02 to 16.0 mM with a detection limit of 0.0056 mM respectively. The demonstrated microbiosensor is a low-cost, robust, highly sensitive, selective, reproducible, and stable with a facile immobilization procedure. Additionally, this biosensor allows the determination of glucose in the presence of ascorbic acid (AA), dopamine (DA), aspartame, fructose, uric acid (UA) and caffeine.

### REFERENCES CITED IN THIS EXAMPLES

[1] S. Marinesco, O. Frey, chapter 10—Microelectrode designs for oxidase-based biosensors in: S. Marinesco, N. Dale (Eds.), Microelectrode Biosensors, Springer, 2013, pp 3-25, https//doi.org/10.1007/978-1-62703-370-1_5.
[2] D. Olczuk, R. A. Priefer, A history of continuous glucose monitors (cgms) in self-monitoring, Diabetes Metab. Syndr.: Clin. Res. Rev, 12 (2018) 181-187, https//doi.org/10.1016/j.dsx.2017.09.005.
[3] B. Bucur, C. Purcarea, S. Andreescu, A. Vasilescu, Addressing the selectivity of enzyme biosensors: solutions and perspectives, review, Sensors 21 (2021) 3038-3074, https//doi.org/10.3390/s21093038.
[4] H. Kaur, M. Shorie, Nanomaterial based aptasensors for clinical and environmental diagnostic applications, Nanoscale Adv. 1 (2019) 2123-2138, https://doi.org/10.1039/C9NA00153K.
[5] N. Varnakavi, L. Nohyun, A review on biosensors and recent development of nanostructured materials-enabled biosensors, Sensors 21 (2021) 1109-1143, https//doi.org/10.3390/s 21041109.
[6] B. Senf, W. H. Yeo, J. H. Kim. Recent advances in portable biosensors for biomarker detection in body fluids, review, Biosensors, 10 (2020) 127, https//doi.org/10.3390/bios 10090127.
[7] T. D. Y. Kozai, A. S. Jaquins-Gerstl, A. L. Vazquez, A. C. Michael, X. T. Cui, Brain tissue responses to neural implants impact signal sensitivity and intervention strategies, ACS Chem. Neurosci. 6 (2015) 48-67, https//doi.org/10.1021/cn500256e.
[8] C. Tan, E. M. Robbins, B. Wu, X. T. Cui, Recent advances in in vivo neurochemical monitoring, Micromachines. 12 (2021) 208-240, https://doi.org/10.3390/mi12020208.
[9] J. Rivnay, S. Inal, B. A. Collins, M. Sessolo, E. Stavrinidou, X. Strakosas, C. Tassone, D. M. Delongcha mp, G. G. Malliaras, Structural control of mixed ionic and electronic transport in conducting polymers, Nat. Commun. 7(2016)₁₁₂₈₇-11296, https//doi.org/10.1038/ncomms 11287.
[10] D. T. Simon, E. O. Gabrielsson, K. Tybrandt, M. Berggren, M. Organic bioelectronics: bridging the signaling gap between biology and technology, Chem. Rev. 116 (2016) 13009-13041, https://doi.org/10.1021/acs.chemrev.6b00146.
[11] M. S. Wilson, Electrochemical immunosensors for the simultaneous detection of two tumor markers, Anal. Chem. 77(2005) 1496-1502, https//doi.org/10.1021/ac0485278.
[12] P. D'Orazio, Biosensors in clinical chemistry, Clin Chim Acta. 334 (2003) 41-69, https//doi.org/10.1016/s0009-8981(03)₀₀₂₄₁-9.
[13] Y. Li, H. J. Schluesener, S. Xu, Gold nanoparticle-based biosensors, Gold Bull. 43(2010) 29-41, https://doi.org/10.1007/BF03214964.
[14] S. Mayilo, M. A. Kloster, M. Wunderlich, A. Lutich, T. A. Klar, A. Nichtl, K. Kürzinger, F. D. Stefani, J. Feldmann, Long-range fluorescence quenching by gold nanoparticles in a sandwich immunoassay for cardiac troponin T, Nano Lett. 9 (2009) 4558-4563, https://doi.org/10.1021/n1903178n.
[15] B. Wu, Y. Kuang, X. Zhang, J. Chen, Noble metal nanoparticles/carbon nanotubes nanohybrids: synthesis and applications, Nano Today. 6 (2011)75-90, https://doi.org/10.1016/j.nantod.2010.12.008.
[16] Lee, I.; Luo, X.; Cui, X. T.; Yun, M. Highly Sensitive Single Polyaniline Nanowire Biosensor for the Detection of Immunoglobulin G and Myoglobin. Biosens. Bioelectron. 2011, 26, 3297-3302.
[17] Izadyar A., Rodriguez K. A., Van, M. N., Tran U., and Hood E. E. “A bienzymatic amperometric glucose biosensor based on using a novel recombinant Mn peroxidase from corn and glucose oxidase with a Nafion membrane”. Journal of Electroanalytical Chemistry 895 (2021) 115387.
[18] I. A. Pavel, S. Lakard, B. Lakard, Flexible Sensors Based on Conductive Polymers, Review: Chemosensors 10 (2022) 97, https://doi.org/10.3390/chemosensors10030097.
[19] P. Si, N. Razmi, O. Nur, S. Solanki, C. M. Pandey, R. K. Gupta, B. D. Malhotra, M. Willander, A, de la Zerda, Gold nanomaterials for optical biosensing and bioimaging, Nanoscale Adv. 3 (2021) 2679-2698, https://doi.org/10.1039/d0na00961j.
[20] A. Izadyar, J. C. Hershberger, R. Robert, Voltammetric assessment of ions transfer at ionophore-graphene based polymeric membranes, Electroanalysis, 30 (2018) 1-5, https://doi.org/0.1002/elan.201800535.
[21] A. Izadyar, Stripping Voltammetry at the Interface between Two Immiscible Electrolyte Solutions: A review, Electroanalysis, 30 (2018) 2210-2221, https://doi.org/10.1002/elan.201800279.
[22] Guzinski M., Jarvis J. M. D'Orazio P., Izadyar A., Pendley B. D., and E. Lindner E. “Solid Contact pH Sensor without CO2 Interference with a Super Hydrophobic PEDOT-C14 as Solid Contact: the Ultimate “Water Layer” Test“. Analytical Chemistry, 2017, 89 (16), 8468-8475.
[23] Izadyar A., Al-Amoody F., Ranawaka Arachchige D. “Ion transfer stripping voltammetry to detect Nanomolar concentrations of Cr (VI) in drinking water” J. Electroanalytical. Chemistry, 2016, 782, 43-49.
[24] Izadyar A., Ranawaka Arachchige D., Cornwell H., and Hershberger J. L. “Ion Transfer Stripping Voltammetry for the Detection of Nanomolar Levels of Fluoxetine, Citalopram, and Sertraline in Tap and River Water Samples” Sensors and Actuators B: Chemical, 2016, 223, 226-233.
[25] Fenoy, G. E.; Giussi, J. M.; von Bilderling, C.; Maza, E. M.; Pietrasanta, L. I.; Knoll, W.; Marmisollé, W. A.; Azzaroni, O. Reversible modulation of the redox activity in conducting polymer nanofilms induced by hydrophobic collapse of a surface-grafted polyelectrolyte. J. Colloid Interface Sci. 2018, 518, 92.
[26] I. Migneault, C. Dartiguenave, M. J. Bertrand, K. C. Waldron, Glutaraldehyde: behavior in aqueous solution, reaction with proteins, and application to enzyme crosslinking, Bio Techniques 37(2004)₇₉₀-802
[27] Woeppel, K. M.; Zheng, X. S.; Schulte, Z. M.; Rosi, N. L.; Cui, X. T. Nanoparticle doped PEDOT for enhanced electrode coatings and drug delivery. Adv. Healthc. Mater. 2019, 8, 1900622.
[28] Hao, Y.; Zhou, B.; Wang, F.; Li, J.; Deng, L.; Liu, Y. N. Construction of Highly Ordered Polyaniline Nanowires and their Applications in DNA Sensing. Biosens. Bioelectron. 2014, 52, 422-426.
[29] Wang J. Electrochemical glucose biosensors. Chem Rev. 2008:108:814-25. doi:10.1021/cr068123a.
[30] Urban G A. Micro- and nanobiosensors—state of the art and trends. Meas Sci Technol. 2008:20:012001. doi: 10.1088/0957-0233/20/1/012001.
[31] Gifford R. Continuous glucose monitoring: 40 years, what we've learned and what's next. ChemPhysChem. 2013; 14:2032-44. doi: 10.1002/cphc.201300172.
[32] Izadyar A., Tran U., and Hood E. E. “Recombinant Mn Peroxidase from Corn Grain Has an Excellent Electrocatalytic Effect in a Designed Amperometric Biosensor to Detect Hydrogen Peroxide at Low Concentrations” ACS Sustainable Chem. Eng. 2019, 7, 19434-19441.
[33] Jarvinen, J.; Taskila, S.; Isomaki, R.; Ojamo, H. Screening of white-rot fungi Manganese peroxidases: a comparison between the specific activities of the enzyme from different native producers, AMB Express 2012, 2, 62.
[34] D. C. Harris, Quantitative Chemical Analysis, 10th ed.; W. H. Freeman and Company: New York, (2020), pp 83-91.
[35] Bard A J and Faulkner L R. Electrochemical Methods: Fundamentals and Applications; John Wiley & Sons, 2nd edn. New York (2001).
[36] R. B. Rakhi, Pranati Nayak, ChuanXia, Husam N. Alshareef “Novel amperometric glucose biosensor based on MXene nanocomposite” Scientific Reports, 2016, 6,36422, DOI: 10.1038/srep36422.
[37] Huangxian Ju, Dongmei Zhou, Yi Xiao, Hongyuan Chen “Amperometric Biosensor for Glucose Based on a Nanometer-Sized Microband Gold Electrode Coimmobilized with Glucose Oxidase and Poly(o-phenylenediamide)” Electroanalysis 1998, 10, 541-545.
[38] Komathi, S.; Gopalan, A. I.; Muthuchamy, N.; Lee, K. P. Polyaniline nanoflowers grafted onto nanodiamonds via a soft template guided secondary nucleation process for high-performance glucose sensing. RSC Adv. 2017, 7, 15342-15351.
[39] Shen, Q.; Zheng, J. Bienzyme system for the biocatalyzed deposition of polyaniline templated by multiwalled carbon nano-tubes: A biosensor design. Biosens. Bioelectron. 2019, 15, 1621-2629.
[40] Lovi'c, J.; Stevanovic, S.; Nikoli'c, N. D.; Petrovi'c, S.; Vukovi'c, D.; Prlainovi'c, N.; Mijin, D.; Avramov Ivi'c, M. Glucose Sensing Using Glucose Oxidase-Glutaraldehyde-Cysteine Modified Gold Electrode. Int. J. Electrochem. Sci 2017, 12, 5806-5817.

### Example 13—Development of a Highly Sensitive Glucose Nanocomposite Biosensor Based on Recombinant Enzyme from Corn (Izadyar et al., J Sci Food Agric 2022, 102: 6530-6538)

Abstract

BACKGROUND: Enzymes are biocatalysts that play a vital role in the production of biomolecules. Plants can be a valuable and cost-effective source for producing well-structured recombinant enzymes. Glucose is one of the most important biological molecules, providing energy to most living systems. Electrochemical method for immobilization of enzyme is promising because it is economic, generates less component waste, improves the signal-to-noise ratio, leads to a lower limit of detection, and stabilizes and protects the enzyme structure.

RESULTS: A glucose biosensor was constructed using polyaniline (PANI) and a recombinant enzyme from corn, Plant-Produced Manganese Peroxidase (PPMP), with polymerization of aniline as a monomer in the presence of gold nanoparticles (AuNPs)—glucose oxidase (GOx), and bovine serum albumin (BSA). Using Linear Sweep Voltammetry (LSV) and Cyclic Voltammetry (CV) techniques, PANI-AuNPs-GOX-PPMP/Au electrode exhibited a superior sensing property with a wider linear range of 0.005 to 16.0 mM, and a lower detection limit (LOD) of 0.001 mM compared to PANI-GOx-PPMP/Au Electrode and PANI-GOX-PPMP/AuNPs/Au Electrode. The biosensor selectivity was assessed by determining glucose concentrations in the presence of ascorbic acid (AA), dopamine (DA). Aspartame, and Caffeine.

OUTCOME: A plant-produced Mn peroxidase enzyme combined with CPs and AuNPs results in a nanocomposite biosensor to detect glucose. The use of such devices for quality control in the food industry can have a significant economic impact.

### INTRODUCTION

Enzymes play a key role in improving various products' physical, chemical, and sensory properties. Recently we applied a recombinant enzyme from corn kernels to fabricate electrochemical-based biosensors for glucose and hydrogen peroxide determination. Chronoamperometric and cyclic voltammetry results showed a great potential of PPMP to meet the growing demands for enzymes in commercial applications.^{1,2}

Glucose metabolism occasionally becomes uncontrolled causing a myriad of health problems. Worldwide, the number of people with diabetes is currently estimated by the International Diabetes Federation at around 500 million,³ and this number is steadily increasing, which puts a heavy burden on the health care system. Therefore, there is still a solid need to lower their cost and enhance biosensor sensitivity, selectivity, stability, simplicity, and accessibility to users. Also, close monitoring of glucose levels in biological samples is essential to ensure that a low amount is present.⁴⁻⁶ Glucose sensors can be enzymatic in nature, such as using glucose oxidase enzyme in combination with enzymes from fungi, for example, the industrially accepted Aspergillus niger enzyme, which is expensive and has low activity.^{7,8}

Our previous work showed that PPMP has excellent activity^{1, 2}, and we fabricated a simple and inexpensive biosensor using this new enzyme and polymeric nanocomposites. Mechanical strength and low reactivity to aging agents are always required for sensor coatings. Because of this, polymeric nanocomposites (PNCs), which offer strength and low reactivity, play an essential role in measuring, processing, and activating electrodes for electrochemical and biosensing applications. To take full advantage of these compositions, they must have good processing capability, which is ultimately driven by conductive polymers (CPs) as one component of the nanocomposites.⁹ π-π conjugated polymers, which can be synthesized via chemical or electrochemical oxidation of monomers, are used to develop rechargeable batteries and sensors.¹⁰⁻¹³ These CPs are characterized by excellent electrical properties, are light weight, efficient, low cost, flexible, have high biocompatibility and act as redox mediators to facilitate the transfer of electrons to the sensor.¹⁴ Polyaniline (PANI) as an CP is studied and utilized because of its easy and inexpensive synthesis: by oxidation of aniline.¹⁵⁻¹⁷ Although PANI is pseudocapacitive, it still suffers from limited cycling stability and high auto-discharge.¹⁸ To solve this limitation. PANI can be combined with various mineral nanomaterials such as gold nanoparticles (AuNPs) as composites.¹⁹⁻²¹

The properties of nanocomposites depend on the shape and volume fraction, the morphology, and the nature of the interphase between the two components.²² AuNPs enhance the sensitivity of a sensor and have many advantages such as a large specific area, biocompatibility, and high chemical stability.²³⁻²⁶

Immobilizing enzymes on an electrode is a method to improve enzyme reusability and stability and enhance the capacity and accessibility of biomolecules.^{27, 28} Immobilization of the target enzyme on conductive nanomaterials facilitates electron transfer in the sensor system, because enzymes by themselves are not conductive. The synthesis performance using an electrochemical technique is fast and easy by anodic oxidation of the monomer onto working electrodes.^{29, 30} In addition, variability of membrane thickness is achieved by adjusting the applied potential during electropolymerization.^{2, 31-33}

Horseradish peroxidase (HRP) with GOx is an enzyme pair for glucose detection. HRP has several disadvantages for glucose detection, including high cost and finite reaction conditions. Here, we used recombinant Mn Peroxidase from corn (plant-produced manganese peroxidase. PPMP) to detect glucose to solve these problems. PPMP is a recombinant fungal manganese peroxidase (MnP)^{1,2} which is produced in a cost-effective plant production system. The enzyme gene (Genbank access #: 129039) was cloned from the white-rot fungus Phanerochaete chrysosporium and expressed recombinantly in corn kernels.³⁴

In this work, we co-immobilized PPMP. AuNPs. and GOx onto a gold electrode by electropolymerization of PANI to demonstrate a stable, sensitive, selective, low-cost, and flexible glucose sensor. Experiments were then performed to validate the effect of substrate flexibility on the performance of the modified electrode, and the PANI film deposition was the only critical parameter. The sensing ability of the thin films with respect to glucose was evaluated by LSV and CV, which show good controllability and reproducibility of this glucose sensor. Characterization of the biosensor membrane was studied by scanning electron microscopy (SEM) and revealed different morphologies of the various composites. Finally, the selectivity and applicability of glucose detection using AA, DA. Aspartame, and caffeine was validated.

Materials and Methods

The glucose stock solution was prepared by mixing a given amount of α-D-glucose anhydrous, 96% from Sigma-Aldrich in phosphate buffer (NaPB; pH 7.0). The prepared stock was diluted to the concentrations. Manganese (II) acetate tetrahydrate 9.99% (Mn(CH3COO)2), bovine serum albumin (BSA), Glucose Oxidase from Aspergillus niger, phosphate buffer solution (PBS) using NaH₂PO₄, and Na₂HPO₄, gold nanoparticles (20 nm, suspension in 0.1 mM PBS), and aniline were purchased from Sigma-Aldrich (St. Louis, MO). All of the solutions were prepared in Milli-Q deionized water (18.2 MΩ) unless otherwise noted. All of the experiments were conducted at laboratory room temperature (20° C.)

Electrode Construction

Modified electrodes were prepared by immobilizing PPMP and GOx by electropolymerizing PANI onto a 5 mm diameter polished gold electrode.¹ A mixer was provided including 0.5 M PPMP, 0.17 M Aniline, 0.10 M GOx, and 2.4 μM BSA in PBS (pH 7.0), and then the Au electrode was immersed into this composite. Polymerizations were conducted by the cyclic voltammetric sweep of 10 to 25 cycles with potential ranging from −0.25 to 0.45 V/Ag/AgCl, at a scan rate of 0.05 Vs⁻¹. Polymer nanocomposites show the best stability using 20 cycles used in all of our electrode modifications.

Apparatus

All voltammetry and amperometric measurements were performed using a computer-controlled CHI660D electrochemical workstation (CH Instruments, Austin, TX). Experiments were carried out in a three-electrode cell with a platinum wire (0.25 mm diameter, 99.9%, Alfa Aesar, Ward Hill, MA) as a counter electrode and a 5 mm-diameter gold electrode as the working electrode, and Ag/AgCl (3 M KCl) as the reference electrode as follows:

Ag/AgCl∥xM(glucose)(aq),0.1 M Phosphate Buffer(pH 7.0) and 0.1 mM(MnSO4|polymer matrix((PPMP(0.5 M)-Aniline(0.17 M)-GOx(0.10 M)-BSA(2.4×10)-6 M)-AuNPs(1.0 g))|Au Cell (1)

A NANO 1000S single wheel grinder timer bench top polisher with variable speed 100-1000 rpm, rapid programmable speed and time selection from Pace Technologies Corporation was applied to polish the gold electrode surface. A benchtop vacuum spin coater (8000 rpm) from MTI Corporation was applied for electrode spin coating. Scanning Electron Microscope (SEM) images were recorded on an SNE-4500 M Plus Tabletop Scanning Electron Microscope Nano Images, LLC.

Results and Discussion

Linear Sweep Voltammetry (LSV) Performance of PANI-Gox-PPMP/Au Electrode

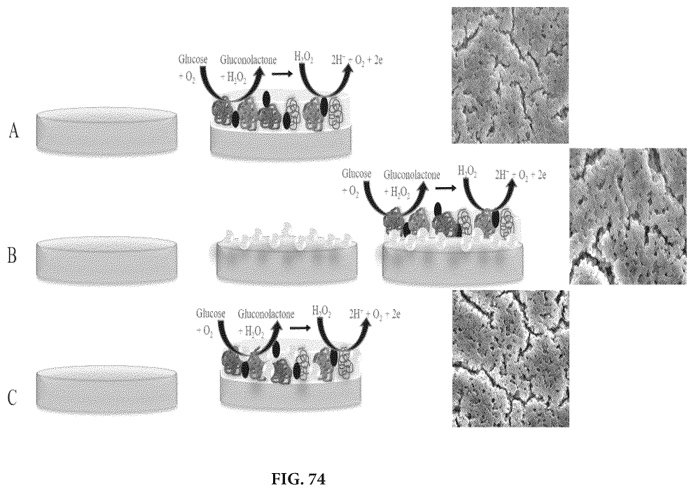

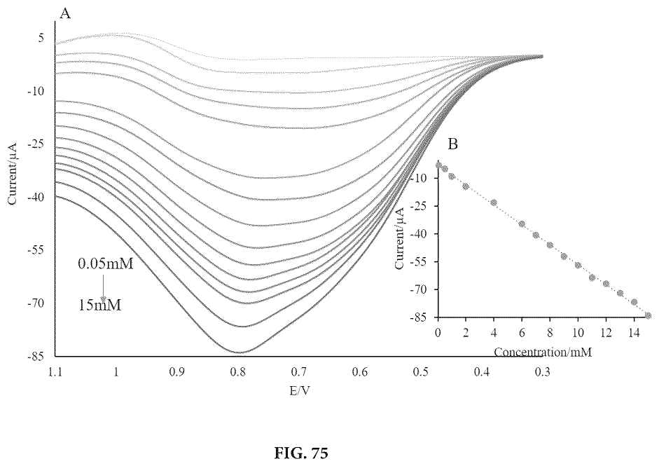

The electropolymerizing of the PANI-GOx-PPMP/Au electrode is characterized by an increase in the anode current corresponding to the oxidation of the aniline monomer to form the polymer and a reduction peak current is observed, attributed to the reduction of the aniline formed during its oxidation. There are two peaks observed in the oxidation and reduction sweep. The anodic peak was observed at −106 mV and at +119 mV, and the cathodic peak was found at −102 mV and 25 mV. The oxidation peak at −106 mV is polyaniline, which is oxidized into a semi-oxidized stat. In comparison, +119 mV is the oxidation peak of the semi-oxidized state, which is oxidized into the fully oxidized form of polyaniline (FIG. 67). The modified electrodes were stored in an incubator at 4° C. when not in use. The process for fabricating the PANI-GOx-PPMP/Au modified electrode is schematically shown in Figure. 74 panel A. To monitor the characteristics of the modified electrode, we applied LSV using a three-electrode system where a platinum wire was used as an auxiliary electrode, an Ag/AgCl electrode as a reference, and the PANI-GOx-PPMP/Au electrode as the working electrode. FIG. 75 panel A represents LSV results of glucose oxidation on the PANI-GOx-PPMP/Au electrode. The LSV was conducted in a 10 mL solution of PBS pH 7 containing 0.1 mM Mn(CH₃COO)₂ in a potential range from 0.1 V to 1.1 V/Ag/AgCl, at a scan rate of 0.05 Vs⁻¹ in an oxygen saturated solution. The detection of glucose was made indirectly by monitoring the oxidation of H₂O₂, since H₂O₂ was released with a reaction of glucose and oxygen in the presence of GOx, and H₂O₂ is reduced to water by the PPMP.

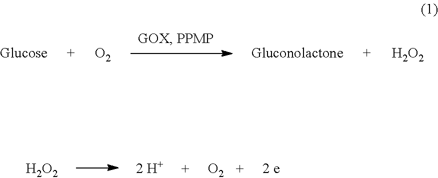

Upon addition of glucose, oxidation peaks increase proportionally at +0.80 V/Ag/AgCl, at a scan rate of 0.05V/s. A sharp increase in current was associated with increasing glucose concentration and peak potential shift to more positive, indicating an increase in the local concentration of oxygen on the surface of the PANI-GOX-PPMP/Au electrode. FIG. 75 panel B shows the calibration plot of the response current to different glucose concentrations of the PANI-GOX-PPMP/Au electrode. Good linear response with increased glucose concentration was obtained within 0.05-15.0 mM with a limit of detection of 0.016 mM. The regression line (R²) values, which fitted well to the calibration curves, were 0.9991. The limit of detections (LOD) was calculated for S/N=3 and S=(3×std dev)+blank. 35 Standard deviation, S, is calculated as shown by Eq. (1) (N is the number of standard solutions). N−2 is the degree of freedom.

$$\\ \begin{matrix}s = \sqrt{\frac{\sum {y}_{i}^{{}_{} 2} - \frac{{( \sum {y}_{i} )}^{2}}{N} - {m}^{2} [ \sum {x}_{i}^{{}_{} 2} - \frac{{( \sum {x}_{i} )}^{2}}{N} ]}{N - 2}} & ( 2 )\end{matrix}$$

The data indicated an obvious electrocatalytic response of PANI-GOX-PPMP/Au electrode and can be used to quantitatively monitor glucose.

Linear Sweep Voltammetry (LSV) Performance of PANI-GOX-PPMP/AuNPs/Au Electrode

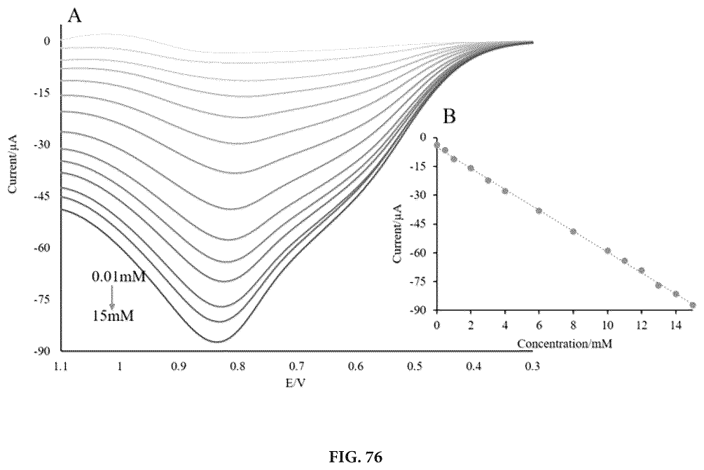

To validate the effect of AuNPs on biosensor performance, we prepared a new PPMP enzyme biosensor based on gold nanoparticles that were directly spin-coated onto the Au electrode. The electrode was stirred at 500 rpm for 5 minutes using the spin coater. Three AuNPs/Au electrodes were prepared with different nanoparticle coverage by controlling the amount of deposition, 2 of 7 μl. 2 of 10 μl, and 2 of 15 μl. In the first stage of coverage, the 7 μl of AuNPs solution composition, the second 7 μl treatment of AuNPs was dropped ten minutes later into the same location, and the same method was performed to apply other amounts. Afterward, a solution containing a mixture of 0.5 M PPMP, 0.17 M Aniline, 0.10 M GOx, and 2.4 μM BSA in PBS (pH 7.0) was prepared. The AuNP coated electrode was immersed into the enzyme solution, and similarly to what has already been described herein, the composite was immobilized with a continuous cyclic voltammetric of 20 cycles on the AuNPs/Au. This electrode was called PANI-GOX-PPMP/AuNPs/Au, and the prepared electrodes were stored in an incubator at 4° C. when not in use. Electrochemical measurements were carried out in a cell containing 10 mL of 0.1 M PBS (pH 7.4) containing 0.1 mM Mn(CH₃COO)₂ at a scan rate of 0.05 Vs⁻¹. We performed LSV on the PANI-GOX-PPMP/AuNPs/Au electrode as the working electrode in the three-electrode system as described herein. The best sensor response was from the electrode that was spin-coated with 2 doses of 7 μl AuNPs. As FIG. 76 panel A shows, in the LSV of the PANI-GOX-PPMP/AuNPs/Au modified electrode, the anodic peak current at 0.83 V corresponds well with the increased glucose concentrations. The PANI-GOX-PPMP/AuNPs/Au electrode has a detection limit of 0.003 mM with a linear range of 0.010-15.0 mM (FIG. 76 panel B). The R² values of the regression lines that fitted to the calibration curves were 0.9991. The results show that the PANI-GOX-PPMP/AuNPs/Au electrode shows greater electrocatalytic activity toward glucose determination than the PANI-GOX-PPMP/Au electrode. More activity and lower LOD are due to an increase in the electrode's conductivity and larger surface area, which exhibits better electron transfer. Furthermore, the higher peak currents of the PANI-GOX-PPMP/AuNPs/Au electrode compared with the PANI-GOX-PPMP/Au electrode exhibits the most effective electron transfer which proves that AuNPs are a great platform to increase enzyme load and effectively improve charge transfer between the glucose and the electrode surface. Peak potential is slightly higher for the PANI-GOX-PPMP/AuNPs/Au electrode than the PANI-GOX-PPMP/AuNPs/Au electrode, which can be due to a change in the mechanism of electron transfer using gold nanoparticles.

Linear Sweep Voltammetry (LSV) and Cyclic Voltammetry (CV) Performance of PANI-GOX-PPMP-AuNPs/Au electrode

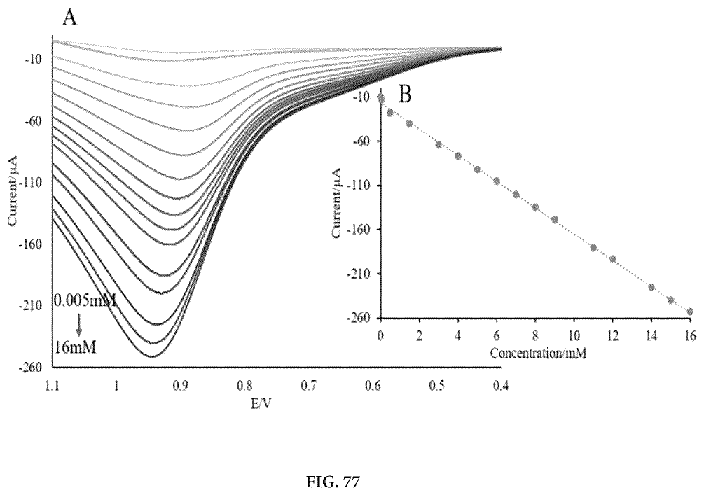

We applied AuNPs in situ directly to the PANI matrix. A 5 mm diameter polished Au electrode was immersed in a solution containing 0.5 M PPMP. 0.17 M Aniline. 0.10 M GOx. 2.4 μM BSA, and 1.0 g AuNPs in PBS (pH 7.0) and electropolymerized as described herein. The electrode was called PANI-AuNPs-GOX-PPMP/Au. and the prepared electrodes were stored in an incubator at 4° C. when not in use. In some embodiments, the cell setup described herein, the PANI-AuNPs-GOX-PPMP/Au electrode was used as the working electrode, a platinum wire was used as an auxiliary electrode, and an Ag/AgCl electrode as reference. Electrochemical measurement was carried out by LSV as described herein to characterize the electrochemical behaviors of the PANI-AuNPs-GOX-PPMP/Au electrode. LSV results (FIG. 77 panel A) show that the oxidation peak of AuNPs is at +0.94 V at a scan rate of 0.05 Vs⁻¹. Good linear response with increased glucose concentration was obtained within a wide range of 0.005-16.0 mM. The R² values of the regression lines that fitted to the calibration curves were 0.9990 with a lower limit of detection of 0.001 mM (FIG. 77 panel B). The PANI-GOX-PPMP-AuNPs/Au electrode exhibits a more comprehensive linear range and a lower detection limit compared to the PANI-GOX-PPMP/AuNPs/Au electrode and the PANI-GOX-PPMP/Au electrode. We observed (FIG. 77 panel A) a higher peak current for this modified electrode due to the conducting nature of AuNPs and the increased active surface area of nanocomposites, which offers more freedom and activity of enzymes.³⁶ The results prove that the larger surface area facilitates the high enzyme loading and lower LOD of PANI-GOX-PPMP-AuNPs/Au electrode compare with PANI-GOX-PPMP/AuNPs/Au electrode. Moreover, the PANI-GOX-PPMP-AuNPs/Au electrode, due to π-π* benzene ring transfer of polyaniline in nanocomposites shows incremental electrical conductivity increases.

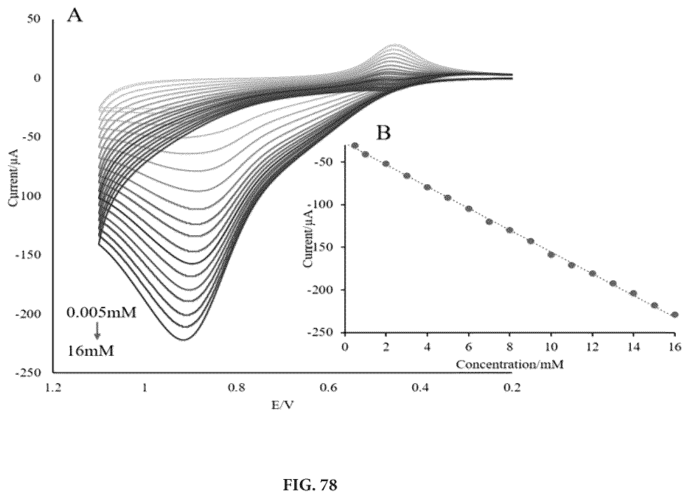

The ability of PANI-GOX-PPMP-AuNPs/Au electrode nanocomposites towards glucose detection was also validated by CV in the same cell setup. FIG. 78 panel A indicates that the oxidation peak at +0.91 V at a scan rate of 0.05 Vs⁻¹ increased significantly with the addition of glucose, indicating the responsivity of the biosensor toward glucose. Furthermore, the gold reduction peak in a cathodic scan appeared at 0.44V. Without wishing to be bound by theory, the change of peak potential of CV (0.91 V) compared with LSV (0.94 V) to a slightly lower voltage value results from changes in AuNPs-mediated electron transfer kinetics. Proportional increases of oxidation peaks with glucose addition were also observed (FIG. 78 panel B). An increase in glucose concentration corresponds with dramatic increases in current and shifted the oxidation peak towards more a positive potential, which indicated a rise in the local concentration of oxygen on the PANI-GOX-PPMP-AuNPs/Au electrode surface. The calibration plot of the current-concentration response curve showed that it has a linear relationship with a correlation coefficient of 0.9991. The designed biosensor possessed an excellent linear response between 0.005-16.0 mM. The LOD of 0.001 mM is much lower than our previous work with Nafion™/PPMP-GOx-PBS/Au electrode, 2 and other published work such as with a as with a PtCo/NPG/GP electrode, 37 GR/PANI-AuNPs(6 nm)-GON/GOx electrode,³⁸ and a AuNPs-decorated MoS₂ nanocomposite electrode.³⁹

Responses of the PANI-GOX-PPMP-AuNPs/Au Electrode at Different Scan Rates

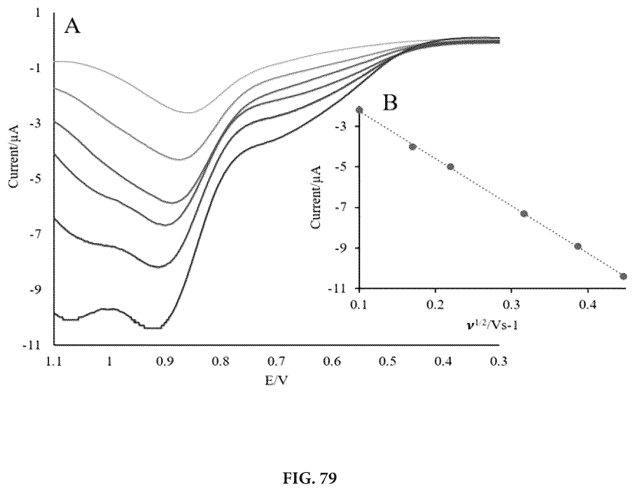

To further characterize our newly constructed electrode, the diffusion-controlled electrochemical behavior was also established by the background-subtracted LSV response of the PANI-GOX-PPMP-AuNPs/Au electrode at different scan rates in the same cell set up (FIG. 6A). The sweep rate was changed from 0.01 to 0.20 Vs⁻¹ in 0.050 mM glucose in PBS pH 7/0.1 mM Mn(CH₃COO)₂ in an oxygen saturated solution. According to the Randles Sevcik equation: 40

$$\\ {i}_{p} = ( 2.687 \times {10}^{5} ) {n}^{\frac{3}{2}} A \cdot {C ( D \cdot v )}^{1 / 2}$$

where iₚ is peak current, n is the number of electrons transferred, A is the electrode surface area, C is molar concentration, D is the diffusion coefficient, and v is the scan rate in Vs⁻¹, the oxidation peak current was proportional to the square root of the scan rates linear relationship with a correlation coefficient of 0.9995 (FIG. 79 panel B). This linear relationship indicates that the electrochemical process was controlled by diffusion and verifies that the electron transfer processes involve freely diffusing glucose. Moreover, there is no significant change in the peak potential, which shows the linear behavior of current as a function of the square root of scan rates.

Selectivity, Stability, and Reproducibility of the PANI-AuNPs-GOx-PPMP/Au Biosensor

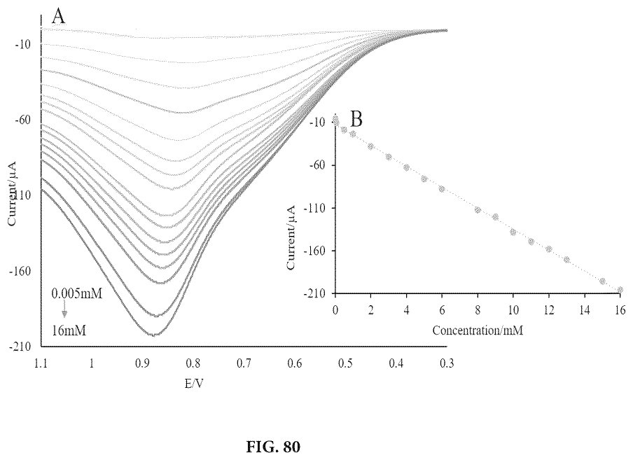

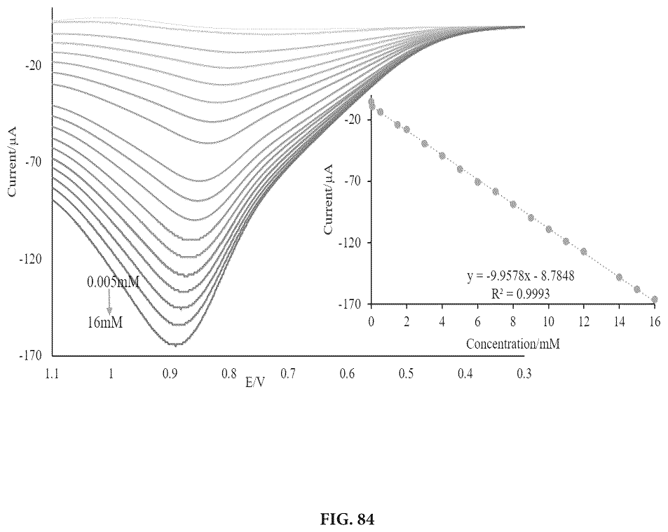

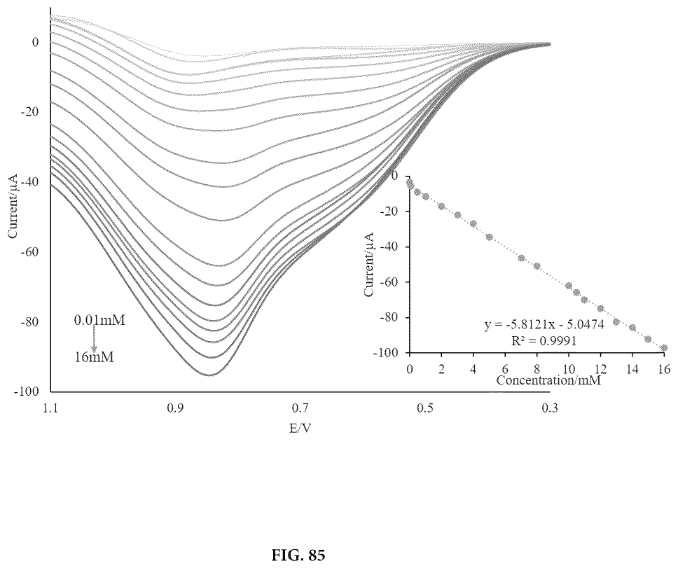

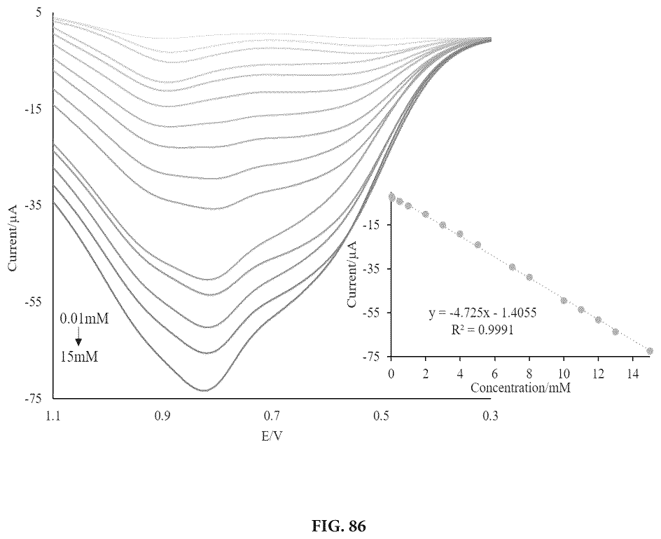

An important characteristic of glucose biosensors is the selective response in the presence of other interfering species. The selectivity of the biosensor was validated in the presence of 0.10 mM AA, DA, Aspartame, and Caffeine. The effect was tested by adding these interferences during glucose measurement using LSV. It was observed that these compounds had no interfering effects on the analysis of glucose. FIG. 80 panel A shows the selectivity of the biosensor in the presence of 0.10 mM DA. A good linear response was obtained within 0.005-16.0 mM (R²=0.9985) with a limit of detection of 0.001 mM (FIG. 80 panel B). A clear current response was observed with the addition of glucose, and negligible effects on the current response were observed when 0.10 mM of AA, Aspartame, or caffeine were added into the solution (FIG. 84-86).

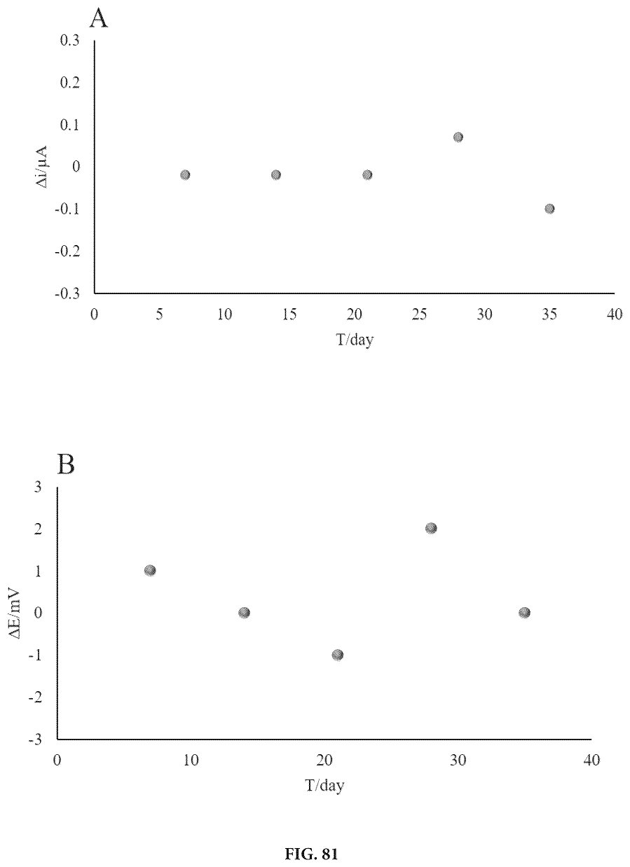

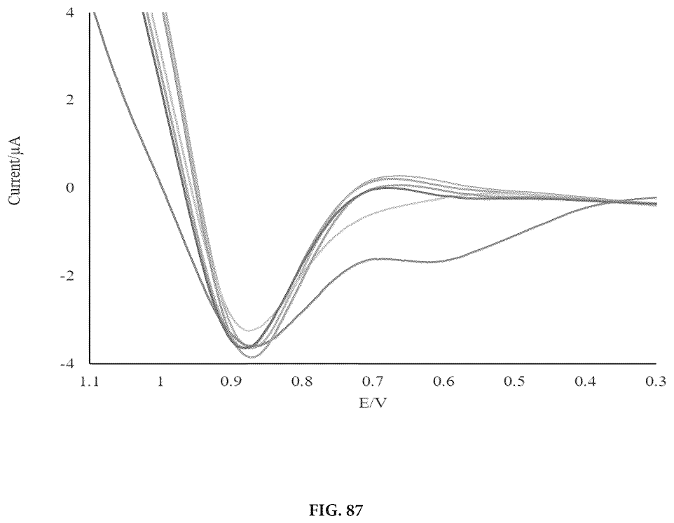

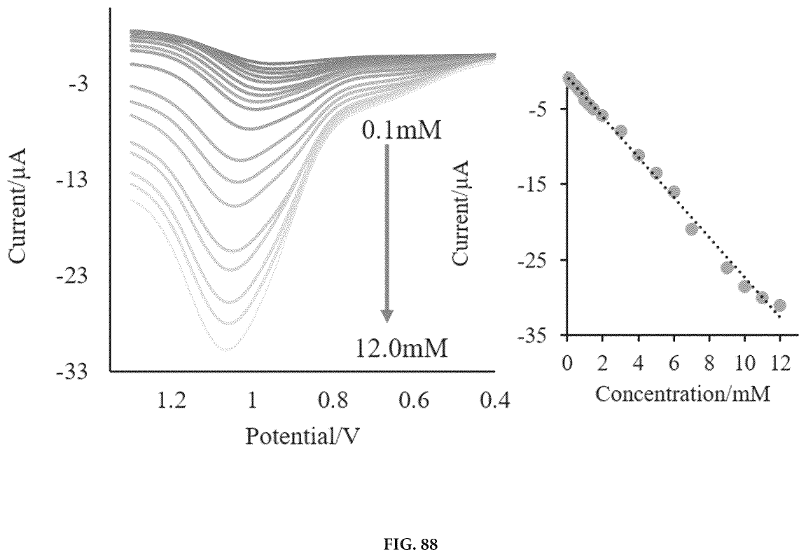

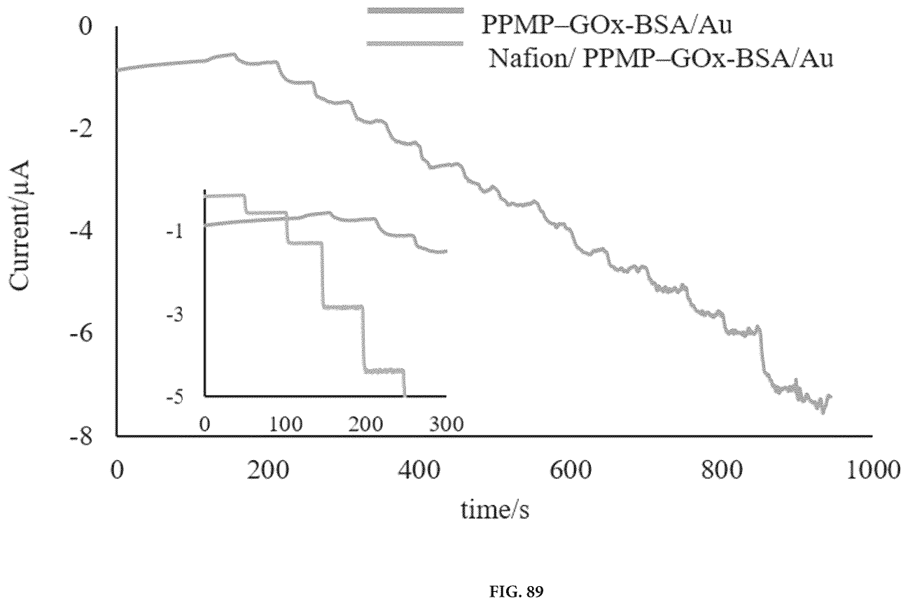

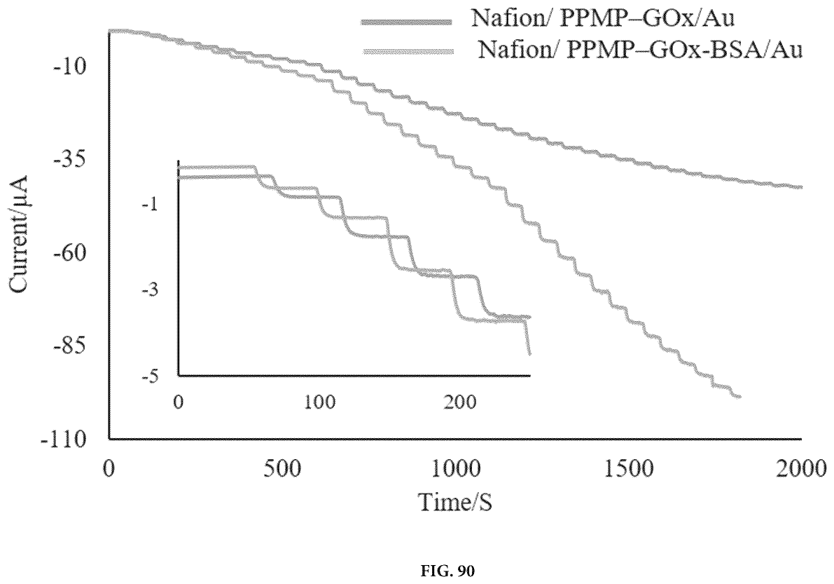

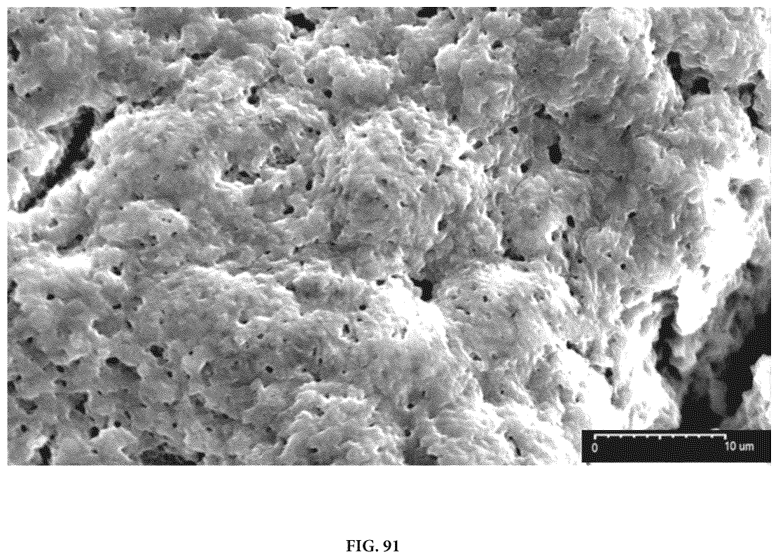

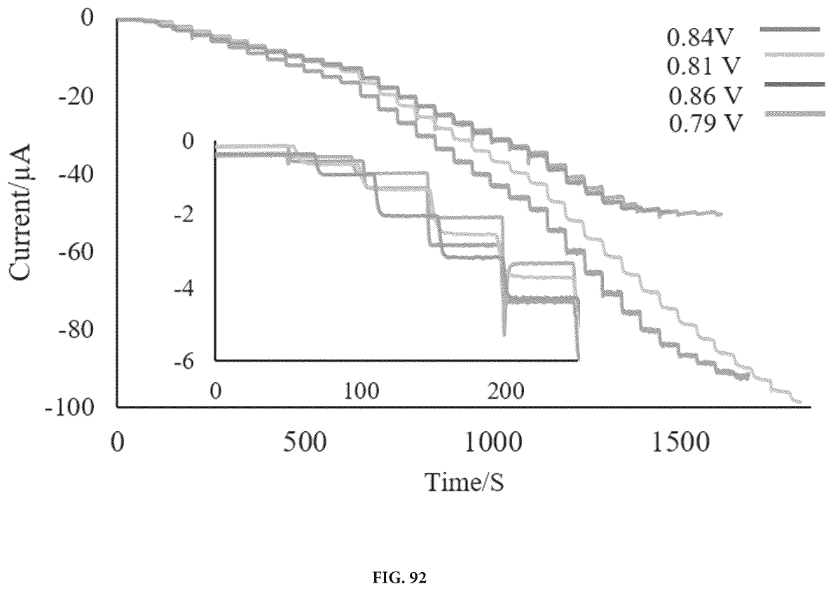

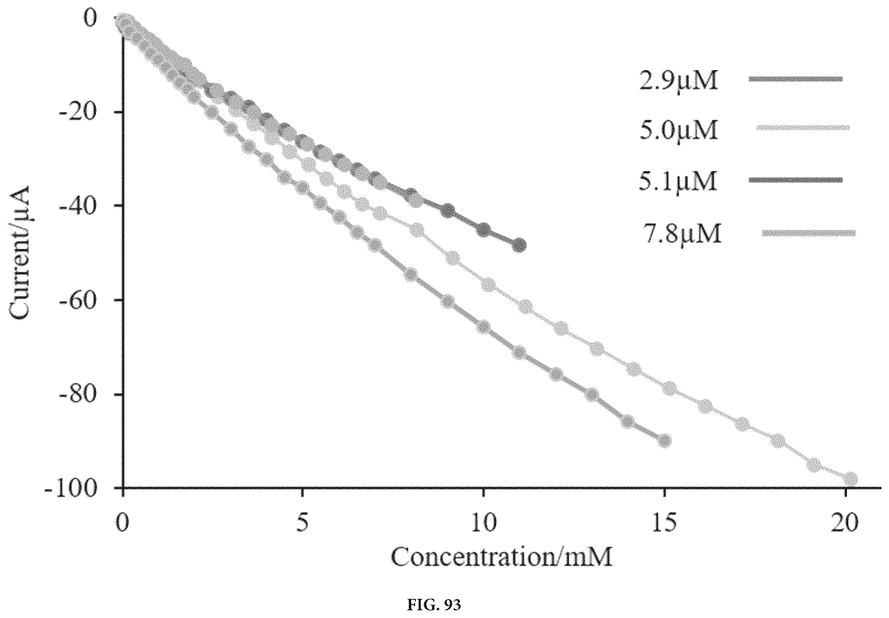

The stability of the PANI-GOx-PPMP-AuNPs/Au glucose biosensor was determined by performing activity assays of PPMP on current within 35 days in 0.05 mM of glucose (FIG. 87). The activity assay was applied within 35 days to determine the storage stability of the immobilized enzyme. As shown in FIG. 81 panel A, an activity loss of 20.0% was observed on the 35th day. We also determined the performance of the PPMP enzyme on potential changes within 35 days. As shown in FIG. 81 panel B, a loss of potential of 0.2% was observed on the 35th day. As a result, it was found that the enzyme was not detached from the electrode surface. Thus, the activity of the enzyme is well protected from the electrode surface.

Morphology Studies

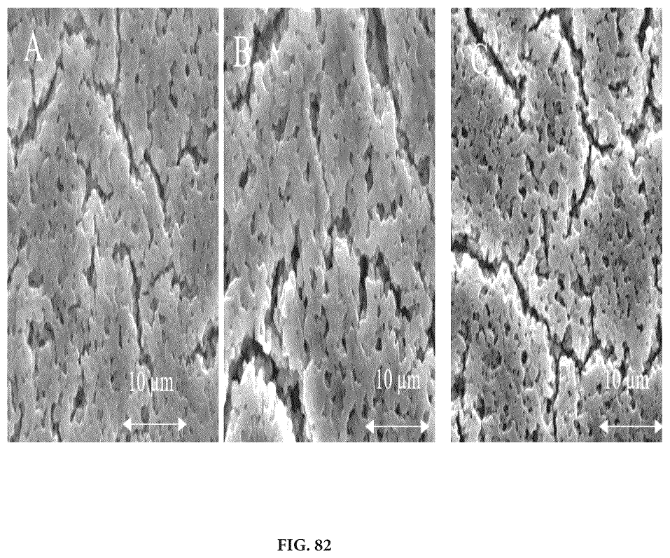

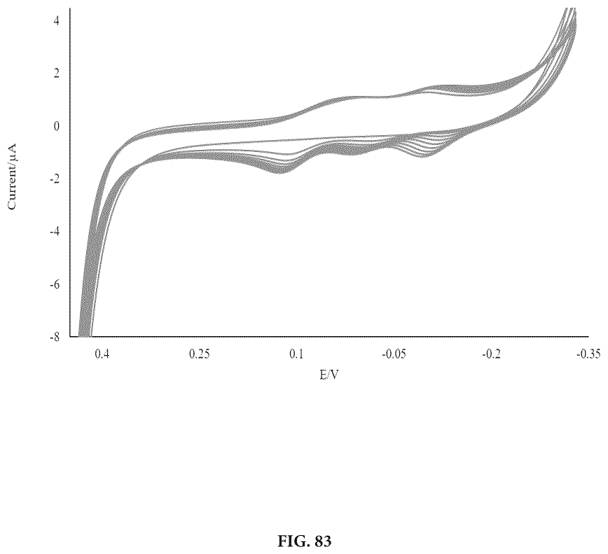

The surface morphology of the PANI-GOx-PPMP-AuNPs/Au electrode compared with the PANI-GOx-PPMP/Au electrode and the PANI-GOx-PPMP/AuNPs/Au electrode were validated through SEM (FIG. 82). The SEM images show a relatively amorphous structure of PANI. The SEM image of the PANI-GOx-PPMP/Au electrode (FIG. 82 panel A) indicates only a polyaniline granular morphology. FIG. 82 panel B illustrates a cauliflower-like morphology of polyaniline for the PANI-GOX-PPMP/AuNPs/Au electrode. In contrast, the SEM image of the PANI-GOX-PPMP-AuNPs/Au electrode (FIG. 82 panel C) indicates a different morphology when the gold nanoparticles are incorporated into the polyaniline matrix during electropolymerization and formation of the nanocomposite. The image shows the porous, closely packed, and concave membrane, allowing for an easy transfer and better conductivity for the PANI-GOX-PPMP-AuNPs/Au electrode.

Outcomes

The results show that the biosensors that contain AuNPs provide a larger surface area, greatly increase the electrocatalytic activity, facilitate high enzyme loading, and lower LOD. Under optimal conditions, the developed PANI-AuNPs-GOX-PPMP/Au electrode exhibits a linear range from 0.005 mM to 16.0 mM with a detection limit of 0.001 mM. The demonstrated biosensor is a reproducible, cost-effective, simple, sensitive, selective, and stable enzyme-based biosensor with a simple immobilization procedure. Additionally, this biosensor allows the determination of glucose in the presence of AA, DA, Aspartame, and caffeine at a concentration of up to 0.1 mM. This biosensor can easily compete with any HRP-based biosensor.

### REFERENCES CITED IN THIS EXAMPLE

1. Izadyar A, Tran U and Hood E E. Recombinant Mn Peroxidase from Corn Grain Has an Excellent Electrocatalytic Effect in a Designed Amperometric Biosensor to Detect Hydrogen Peroxide at Low Concentrations. ACS Sustain. Chem. Eng. 7: 19434-19441, (2019).
2. Izadyar A, Rodriguez K A, Van M N, Tran U and Hood E E. A Bienzymatic Amperometric Glucose Biosensor Based on Using a Novel Recombinant Mn Peroxidase from Corn and Glucose Oxidase with a Nafion Membrane. J. Electroanal. Chem. 895: 115387(2021).
3. Lin X. Xu Y, Pan X, Xu J, Ding Y, Sun X Song X, Ren Y and Shan P F. Global, regional, and national burden and trend of diabetes in 195 countries and territories: an analysis from 1990 to 2025. Sci. Rep. 10: 14790 (2020).
4. Bordonaba J G and Terry L A. Development of a Glucose Biosensor for Rapid Assessment of Strawberry Quality: Relationship between Biosensor Response and Fruit Composition. J. Agric. Food Chem. 57: 8220-8226 (2009).
5. Goriushkina T B, Soldatkin A P and Dryadevych S V. Application of Amperometric Biosensors for Analysis of Ethanol, Glucose, and Lactate in Wine. J. Agric. Food Chem. 57: 6528-6535 (2009).
6. Yoon H, Nah J, Kim H, Ko S, Sharifuzzaman M, Barman S C, Xuan X, Kim J and Park J Y. A chemically modified laser induced porous graphene based flexible and ultrasensitive electrochemical biosensor for sweat glucose detection. Sens Actuators B Chem. 311: 127866 (2020).
7. Zhu T, Wang X, Chang W, Zhang Y, Maruyama T, Luo L and Zhao X. Green fabrication of Cu/rGO decorated SWCNT buckypaper as a flexible electrode for glucose detection. Mater. Sci. Eng. C. 120: 111757 (2021).
8. Nordin N, Bordonali L, Davoodi, H.; Ratnawati, N. D.; Gygli, G.; Korvink, J. G.; Badilita, V.; Mackinnon, N. Real-time NMR monitoring of spatially segregated enzymatic reactions in multilayered hydrogel assemblies. Angew. Chem 133: 19325 (2021).
9. Jianyong O. Application of intrinsically conducting polymers in flexible electronics. SmartMat 2:263-285 (2021).
10. Freund M S and Deore B A. Self-Doped Conducting Polymers; John Wiley & Sons, 1st edn. Chichester, England (2007).
11. Chou J C, Lin S H, Lai T Y, Kuo P Y, Chih-Hsien Lai C H, Nien Y H and Su T Y. A Facile Fabrication of a Potentiometric Arrayed Glucose Biosensor Based on Nafion-GOx/GO/AZO. Sensors 20: 964-984 (2020).
12. Ding Y, Liang J, Liu G, Ni W and Shen L. Preparation and Anticorrosive Property of Soluble Aniline Tetramer. Coatings 9: 399, (2019).
13. Huang W Y, Chang M Y, Wang Y Z, Huang Y C, Ho K S, Hsieh T H and Kuo Y C. Polyaniline Based Pt-Electrocatalyst for a Proton Exchanged Membrane Fuel Cell. Polymers 12:617(2020).
14. Kannan P and Maduraiveeran G. Review, Bimetallic Nanomaterials-Based Electrochemical Biosensor Platforms for Clinical Applications. Micromachines 13: 76 (2022).
15. Alcocer Á T, Plascencia F B, Morillo C C and Cando A P. Review, Electrochemical Sensors Based on Conducting Polymers for the Aqueous Detection of Biologically Relevant Molecules. Nanomaterials 11: 252 (2021).
16. Mazeiko V, Kausaite-Minkstimiene A, Ramanavicienea A and Balevicius Z. Gold Nanoparticle and Conducting Polymer-Polyaniline-Based Nanocomposites for Glucose Biosensor Design. Sens. Actuators B Chem 189:187-193 (2013).
17. Kim H J, Lee J S, Park J P, Lee S, Hong S J, Park J S and Park K P. Fabrication of Nanocomposites Complexed with Gold Nanoparticles on Polyaniline and Application to their Nerve Regeneration. ACS Appl. Mater. Interfaces 12: 30750-30760 (2020).
18. Bogdanovic U, Pasti I, Marjanovic G C, Mitric M, Ahrenkiel S P and Vodnik V. Interfacial Synthesis of Gold-Polyaniline Nanocomposite and Its Electrocatalytic Application. ACS Appl. Mater. Interfaces 7: 28393-28403 (2015).
19. Han J, Li L and Guo R. Novel Approach to Controllable Synthesis of Gold Nanoparticles Supported on Polyaniline Nanofibers, Macromolecules 43: 10636-10644 (2010).
20. Zhang R C, Sun D, Zhang R, Lin W F, Montero M M, Patel J, Askari S, McDonald C, Mariotti D and Maguire P. Gold nanoparticle-polymer nanocomposites synthesized by room temperature atmospheric pressure plasma and their potential for fuel cell electrocatalytic application. Sci. Rep 7: 46682 (2017).
21. Liu D, Wang X, Deng J, Zhou C, Guo J and Liu P. Crosslinked Carbon Nanotubes/Polyaniline Composites as a Pseudocapacitive Material with High Cycling Stability. Nanomaterials 5: 1034-1047 (2015).
22. Jin X, Li G, Xu T, Su L, Yan D and Zhang X. Ruthenium-based Conjugated Polymer and Metal-organic Framework Nanocomposites for Glucose Sensing. Electroanalysis 8: 1902-1910 (2021).
23. Wang S Z, Zheng M, Zhang X, Zhuo M P, Zhou Q, Su Y, Min Zheng M, Yuan G and Wang Z S. Flowerlike CuO/Au Nanoparticle Heterostructures for Nonenzymatic Glucose Detection. ACS Appl. Nano Mater 4: 5808-5815 (2021).
24. Koh W C A, Chandra P, Kim D M and Shim Y B. Electropolymerized self-assembled layer on gold nanoparticles: detection of inducible nitric oxide synthase in neuronal cell culture. Anal. Chem 83: 6177-6183 (2011).
25. Dey G R and Saha A. Surface Engineered PLGA Nanoparticle for Threshold Responsive Glucose Monitoring and “Self-Programmed” Insulin Delivery, ACS Biomater. Sci. Eng 7: 4645-4658 (2021).
26. Naresh V and Lee N. A Review on Biosensors and Recent Development of Nanostructured Materials-Enabled Biosensors. Sensors 21: 1109 (2021).
27. Wang H, Wang X, Zhang X, Qin X, Zhao Z, Miao Z, Huang H and Chen Q. A novel glucose biosensor based on the immobilization of glucose oxidase onto gold nanoparticles-modified Pb nanowires. Biosens. Bioelectron 25:142-146 (2009).
28. Manesh K M, Santhosh P, Gopalan A I and Lee K P. Silica-Polyaniline Based Bienzyme Cholesterol Biosensor: Fabrication and Characterization. Electroanalysis 20: 2467-2474 (2010).
29. Cando A P, Enriquez I R, Tausch M and Scherf U. Thin Functional Polymer Films by Electropolymerization, Nanomaterials 9: 1125 (2019).
30. Izadyar A, Hershberger J C and Robert R. Voltammetric Assessment of Ions Transfer at Ionophore-Graphene Based Polymeric Membranes. Electroanalysis 30: 2580-2583 (2018).
31. Tonelli D, Scavetta E and Gualandi I. Review Electrochemical Deposition of Nanomaterials for Electrochemical Sensing. Sensors 19: 1186-1214 (2019).
32. Guzinski M, Jarvis J M, Orazio P D, Izadyar A, Pendley B D and Lindner E. Solid Contact pH Sensor without CO2 Interference with a Super Hydrophobic PEDOT-C14 as Solid Contact: The Ultimate “Water Layer” Test“. Anal. Chem 89: 8468-8475 (2017).
33. Beaver K, Dantanarayana A and Minteer S D. Materials Approaches for Improving Electrochemical Sensor Performance. J. Phys. Chem. B. 125: 11820-11834 (2021).
34. Jarvinen J, Taskila S, Isomaki R and Ojamo H. Screening of white-rot fungi Manganese peroxidases: a comparison between the specific activities of the enzyme from different native producers. AMB Express 2: 62 (2012).
35. Harris D C. Quantitative Chemical Analysis, W. H. Freeman. 10th end. New (2020).
36. Vilian A T E and Chen, S. M. Direct electrochemistry and electrocatalysis of glucose oxidase based poly(L-arginine)-multi-walled carbon nanotubes. RSC Adv 4: 50771-50781 (2014).
37. Zhao A, Zhang Z, Zhang P, Xiao S, Wang L, Dong Y, Yuan H, Li P, Sun Yand Jiang X. 3D nanoporous gold scaffold supported on graphene paper: Freestanding and flexible electrode with high loading of ultrafine PtCo alloy nanoparticles for electrochemical glucose sensing. Anal. Chim. Acta 938: 63-71(2016).
38. German N, Ramanaviciene A and Ramanavicius A. Dispersed Conducting Polymer Nanocomposites with Glucose Oxidase and Gold Nanoparticles for the Design of Enzymatic Glucose Biosensors, Polymers 13: 2173 (2021).
39. Su S, Sun H, Xu F, Yuwen L, Fan C and Wang L. Direct electrochemistry of glucose oxidase and a biosensor for glucose based on a glass carbon electrode modified with MoS₂ nanosheets decorated with gold nanoparticles. Microchim Acta 181: 1497-1503 (2014).
40. Bard A J and Faulkner L R. Electrochemical Methods: Fundamentals and Applications; John Wiley & Sons, 2nd edn. New York (2001).

### Example 14—Square Wave Electrochemical Voltammetry Study of Recombinant Mn Peroxidase from Corn on Disposable Screen Printed to Detect Glucose

We applied modified screen-printed electrodes (SPEs) in this work using the square wave electrochemical voltammetry (SWV) method. SWV has been widely used in developing electrochemical sensors and biosensors in recent years due to selectivity and high sensitivity. In addition. SPEs are cost-effective, easy to use, and the key to fast and efficient data collection. They can be used for long-term and real-time monitoring and efficient devices for rapid measurement in one step without fear of contamination. We use a recombinant enzyme derived from corn and a modification, a disposable, facile, fast, and cost-beneficial new SPE applying SWV to measure glucose. The Plant Produced Mn Peroxidase (PPMP)-glucose oxidase (GOx)-Bovine serum albumin (BSA). Gold Nanoparticles (GNp) 10 um/gold Screen printed electrode (GSPE) showed an excellent electrochemical response to detect glucose. This biosensor using SWV can respond to a wide range of glucose. As a result, the new disposable sensor is offered as a tool that facilitates medical diagnosis and monitoring of glucose levels in foods.

Electrodeposition Procedure

We applied the electrochemical method to clean SPE using cyclic voltammetry in 1.0 M H2SO4, and PBS. Composite drop cast on the WE surface and electropolymerized by sweeping with potential between −0.10 to +1.6 V at a scan rate of 0.05 Vs⁻¹ for 30 to 15 cycles. The nanocomposite film shows the best stability using 25 cycles in all electrode modifications. We performed Electropolymerization on SPEs using 0.50 M of PPMP. 0.17 M of aniline. 0.250 M of GOx, GNPs (3.04 mg/mL in H₂O, 10 nm), and (2.4×10⁻⁶ M) BSA in PBS (pH 7.0) solution. The modified SPE was called PANI-GNPs-GOX-PPMP/SPE, and the prepared electrodes were stored in an incubator at four ° C. when not in use.

Results and Discussion

Electrochemical Sensing of PANI-GNPs-Gox-PPMP/SPE

We applied Square Wave Voltammetry (SWV) techniques to study the electrochemical activity of PANI-GNPs-GOx-PPMP/SPE techniques to investigate the electrochemical sensing behavior of the modified SPE.

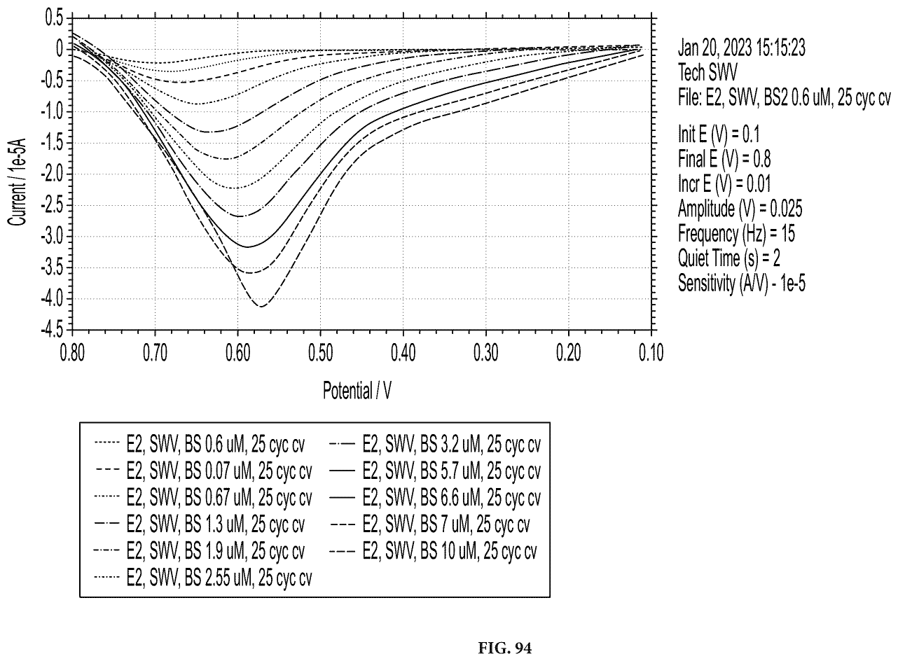

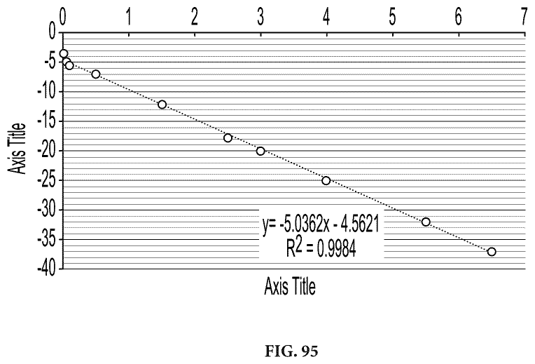

The quantitative determination of glucose in a PBS (pH 7.0)/0.1 mM Mn (CH₃COO)₂ solution saturated with oxygen gas was carried out using SWV technique with the PANI-GNPs-GOx-PPMP/SPE. FIG. 94 shows the background-subtracted swv with potential sweeping between 0.10 to 0.80 V with the successive addition of glucose in the range of 0.0006-6.5 mM, indicating an increase in current at 0.58V potential at a scan rate of 0.05 Vs⁻¹. The change in the oxidation peak potential to a higher value indicates an increase in local oxygen concentration at the PANI-GOx-GNPs-PPMP/SPE surface. The regression line (R²=0.9984) values were fitted to the calibration curves (FIG. 95). The limit of detection (LOD) of 0.29 μM was calculated for S/N=3 and S=(3×std dev)+blank. SWV is used for electroanalysis, can be in aqueous solvents, as they can be an order of magnitude more sensitive than Linear sweep voltammetry (LSV), and the detection limit of SWV is typically superior to LSV. Electrodes were modified as described above, and the voltammetric parameters for SWV were optimized to obtain a prominent peak-shaped voltammogram.

### Example 15

Electrocatalytic Effect of Recombinant Mn Peroxidase from Corn on Microbiosensors to Detect Glucose

Abstract

Our previous studies revealed that a recombinant enzyme from corn is effective and suitable for glucose monitoring. However, the challenge is to further extend our previous work by modification of a 10 μm diameter gold microelectrode. (GME), which guides this biosensor towards practical applications and in vivo analytical monitoring of glucose. The polyaniline (PANI)—gold nanoparticles (GNPs)—glucose oxidase (GOx)-plant-produced manganese peroxidase (PPMP)/GME composition showed excellent chronoamperometric (CA) response for the selective and sensitive enzymatic detection of glucose. PANI-GNPs-GOX-PPMP/GME exhibits a wide linear range of 0.001 to 16.0 mM, and a lower detection limit of 0.50 μM when applying CA. Linear sweep voltammetry (LSV) and cyclic voltammetry (CV) were also applied for further electrochemical characterization of the modified microelectrode to detect glucose. The biosensor selectivity was assessed by determining glucose concentrations in the presence of ascorbic acid, dopamine, aspartame, fructose, uric acid, caffeine, and 10% diet coke. As a result, the new dual enzyme microbiosensor is proposed as a tool that facilitates medical diagnosis and monitoring of glucose levels of foods.

### Introduction

In recent decades, electrochemical biosensors have received tremendous attention and are mainly focused on the healthcare industry. Clinical trials and research efforts have shown that monitoring has a high potential for providing early signs of various disorders and diseases. With this in mind, the development of biosensors for glucose monitoring has become the subject of intense scientific effort for various research groups around the world (Error! Reference source not found, al., 2013: Olczuk et al., 2018). Enzymatic biosensors are now widely used to monitor the concentration of molecules in vivo due to fast and cost effective diagnostic devices. Enzyme-based biosensors have the largest commercial market in diagnostics and are widely studied. The selectivity of enzymatic biosensors is influenced by design parameters and materials used to create the biosensors, modifiers of those materials, and enzymatic stabilization methods to the performance of biosensors (Bucur et al., 2021).

Today, we enjoy the results of nanotechnology for a hassle-free life. Nanotechnology also helps with the performance of sensors and biosensors (Kaur et al., 2019). Extensive studies are underway to make biosensors smaller and more portable in various applications, such as medical diagnostics, environmental monitoring, healthcare and industrial production (Varnakavi et al., 2021; Senf et al., 2020), to increase their spatial resolution, their selectivity, in vivo (Kozai et al., 2015; Tan et al., 2021). Microscale enzyme electrodes that work in small volumes, have attracted more attention due to increasing the signal-to-noise ratio and reducing the sample size. In order to create better miniature biosensors, a large quantity of enzyme must be immobilized on the microelectrode. It is common to use nanomaterials as a solid support to collect more enzymes on the electrode surface due to their large surface area. Additionally, nanomaterials have unique advantages of direct and fast electron transfer between enzyme active sites and the electrode. (Rivnay et al., 2016; Simon et al., 2016), which reduces the response time and improves accuracy. GNPs are popular and play important roles in biosensors (Wilson et al., 2005). These nanoparticles are very convincing for biomedical studies due to their unique properties.

Electrochemical biosensors are a very powerful electroanalytical technique that has the advantage of high sensitivity, simplicity, portability, easy downsizing and low cost. Because of these properties, researchers have made great efforts to improve them, and one of the ways is to include nanomaterials in their construction. Due to the importance of glucose detection for patients with diabetes, many glucose sensors have been developed in the past 3 decades. However, several challenges associated with developing a small device that can quickly, and reliably monitor glucose in vivo remain. Although several different techniques are available, electrochemical methods are the most common and trustworthy techniques for continuous sugar monitoring (Izadyar et al., 2021). Amperometric biosensors are integrated devices where the biochemical receptor is maintained in direct contact with the electrochemical transmission element. Enzyme-based electrochemical bioassays are robust, selective, sensitive with excellent detection limits and the ability to be used for real-time monitoring of complex samples (Wilson et al., 2005).

Flexible sensors with incorporated conductive polymers have attracted a lot of attention because of their potential to be used in different fields (Pavel et al., 2022). Conductive polymers (CPs) have been widely used to improve biosensors by increasing the effective surface area, reducing the impedance, and improving the bioelectronics signal. CPs can be easily obtained by electrochemical oxidation of aromatic monomers on the electrode surface to make a homogeneous thin film (Razmi et al., 2021: Izadyar et al., 2018; Izadyar et al., 2018; Guzinski et al., 2017; Izadyar et al., 2016; Izadyar et al., 2016; Fenoy et al., 2018: Woeppel et al., 2019). Error! Reference source not found. Conductive polymer nanomaterials have significantly useful physical and chemical properties and high potential for the production of cost-effective, large-scale, lightweight, flexible biosensors, which can improve in vivo usage due to antifouling biosensing electrochemical interfaces. Nanobiosensors based on PANI nanomaterials have shown high performance sensory properties toward biological targets and exhibit excellent properties such as long-lifetimes and no toxicity (Gifford et al., 2013; Izadyar et al., 2019).

Horseradish peroxidase (HRP) and GOx are the most common enzyme pair for glucose detection. However, HRP has several disadvantages including high cost and finite reaction conditions. In our previous work (Izadyar et al., 2018; Izadyar et al., 2019), we used plant-produced manganese peroxidase from corn (PPMP) to produce a successful cost-effective dual enzyme biosensor. The enzyme gene (Genbank access #: 129039) was cloned from the white rot fungus Phanerochaete chrysosporium and expressed recombinantly in corn kernels (Jarvinen et al., 2012).

Described herein, we use PPMP, GOx, GNPs, and PANI to construct a new microbiosensor to measure glucose using an electrochemical technique. We suggest a one-step PANI electropolymerization procedure using PPMP. GOx and GNPs on a 10 μm GME. The main purpose of this study was to further explore analytical applications of plant-produced manganese peroxidase based biosensors towards developing an economical, rapid, and appropriate method for determining the concentration of the biologically important compound, glucose. Under the optimum operational conditions, the PANI-GNPs-GOX-PPMP/GME showed a good sensitivity, selectivity, rapid response time and excellent operational stability to monitor glucose. Further, this unique fabricated micro scale electrode will lead to the use of this electrochemical microbiosensor for in vivo applications.

Materials and Methods

2.1 Chemicals

PPMP was prepared as described previously (Izadyar et al., 2019). Error! Reference source not found. Manganese (II) acetate (Mn(CH₃COO)₂), BSA (Bovine serum albumin) GOx from Aspergillus niger. Type X-S, phosphate buffer solution (PBS) using NaH₂PO₄, and Na₂HPO₄, GNPs (ultra-uniform, 10 nm, 0.05 mg/mL (aqueous 2 mM sodium citrate)), aniline, ascorbic acid (AA), dopamine (DA), aspartame, fructose, uric acid (UA), and caffeine were purchased from Sigma-Aldrich (St. Louis, MO). Diet coke was purchased at a local grocery store. All of the solutions were prepared in Milli-Q deionized water (18.2 MΩ) unless otherwise noted. All of the experiments were conducted at laboratory room temperature (20° C.).

2.2 Apparatus

All voltammetry and amperometric measurements were performed using a computer-controlled CHI660D electrochemical workstation (CH Instruments, Austin, TX). Experiments were carried out in a three-electrode cell with a platinum wire (0.25 mm diameter, 99.9%, Alfa Aesar, Ward Hill, MA) as a counter electrode and a 10.0 μm diameter GME as the working electrode, and Ag/AgCl (3 M KCl) as the reference electrode.

Ag/AgCl|xM(glucose),0.1 M PBS(pH 7.0),0.1 mM(Mn(CH₃COO)₂)|polymer matrix((PPMP(0.5 M)-aniline(0.17 M)-GOx(0.10 M)-BSA(2.4×10⁻⁶ M)-GNPs(1.0 g))|GME(10 μm) (cell 1)

2.3 Electrodeposition Procedure

The electrodeposition modification procedure was optimized to reach the maximum sensitivity. A 10 μm diameter gold microelectrode was cleaned using an electrochemical method by cyclic voltammetry in 1.0 M H₂SO₄ and it was sweeping with potential between −0.30 to +1.2 V/Ag/AgCl, at a scan rate of 0.05 Vs⁻¹ for 30 cycles, then the microelectrode cleaned with the same conditions in PBS. Electropolymerization depositions on GMEs were performed using 0.50 M of PPMP, 0.17 M of aniline, 0.10 M of GOx, and (2.4×10⁻⁶ M) BSA in PBS (pH 7.0) solution by cycling of 15 to 30 cycles with potential between −0.42 to 0.50 V/Ag/AgCl, at a scan rate of 0.05 Vs⁻¹. The nanocomposite film shows the best stability using 25 cycles in all electrode modifications. The modified GME was called PANI-GNPs-GOX-PPMP/GME and the prepared electrodes were stored in an incubator at 4° C. when not in use.

3. Results and Discussion

3.1 Electrochemical Sensing of PANI-GNPs-Gox-PPMP/GME

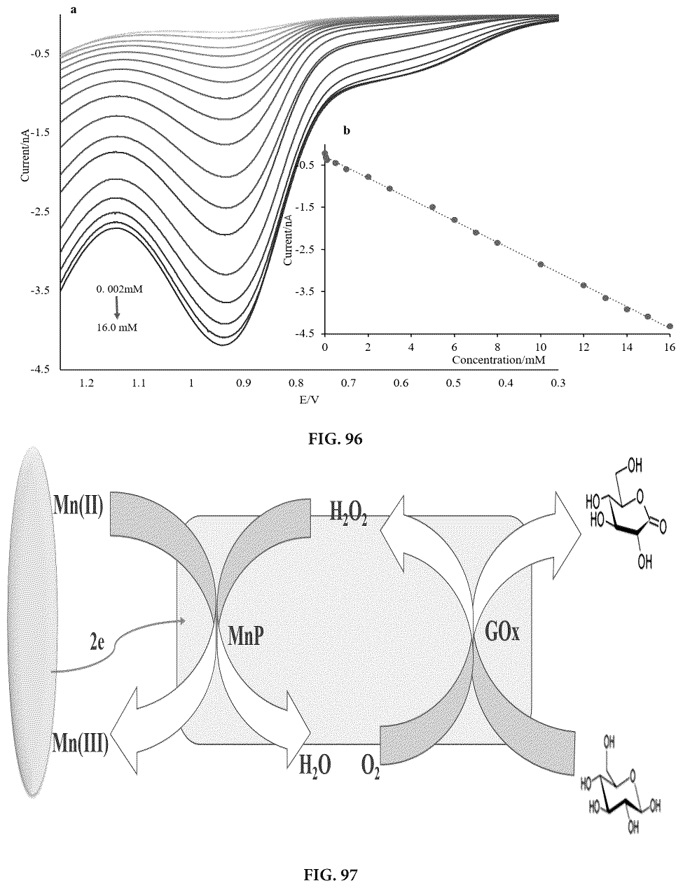

The electrocatalytic activity of PANI-GNPs-GOx-PPMP/GME was studied with the LSV, CV and CA techniques in order to investigate the electrochemical sensing behavior of the modified microelectrode. The quantitative determination of glucose in a PBS (pH 7.0)/0.1 mM Mn (CH₃COO)₂ solution saturated with oxygen gas was carried out using an LSV technique with the PANI-GNPs-GOx-PPMP/GME. FIG. 96 panel a shows the non-background-subtracted LSVs with the successive addition of glucose in the range of 0.002-16.0 mM indicating sharp increase in current at 0.94V potential at a scan rate 0.05 Vs⁻¹. The change in the oxidation peak potential to a higher value indicates an increase in local oxygen concentration at the surface of the PANI-GOx-GNPs-PPMP/GME electrode. Error! Reference source not found. The regression line (R²=0.9993) values were fitted to the calibration curves (FIG. 96 panel b). The limit of detection (LOD) of 0.6 μM was calculated for S/N=3 and S=(3×std dev)+blank (Harris (2020))). This result demonstrates an excellent electrocatalytic performance of PANI-GOx-GNPs-PPMP/GME, which is due to the catalytic activity of the PPMP enzyme. The high surface area of GNPs allows immobilization of a large number of enzyme molecules, which significantly increases the sensitivity of the sensor, and provides a superior electron transfer capability.

Using PANI-GOx-GNPs-PPMP/GME, glucose is quantified by the electrochemical measurement of hydrogen peroxide. Peak currents are based on consumption of oxygen by the enzyme-catalyzed reaction. The coimmobilization of PPMP and GOx made enzymatically produced H₂O₂ immediately available for reduction by manganese peroxidase (MnP) using the PPMP enzyme. PPMP is electrically connected to the electrode surface which, amplified the electrochemical responses and enhanced the sensitivity of the biosensor (FIG. 97).

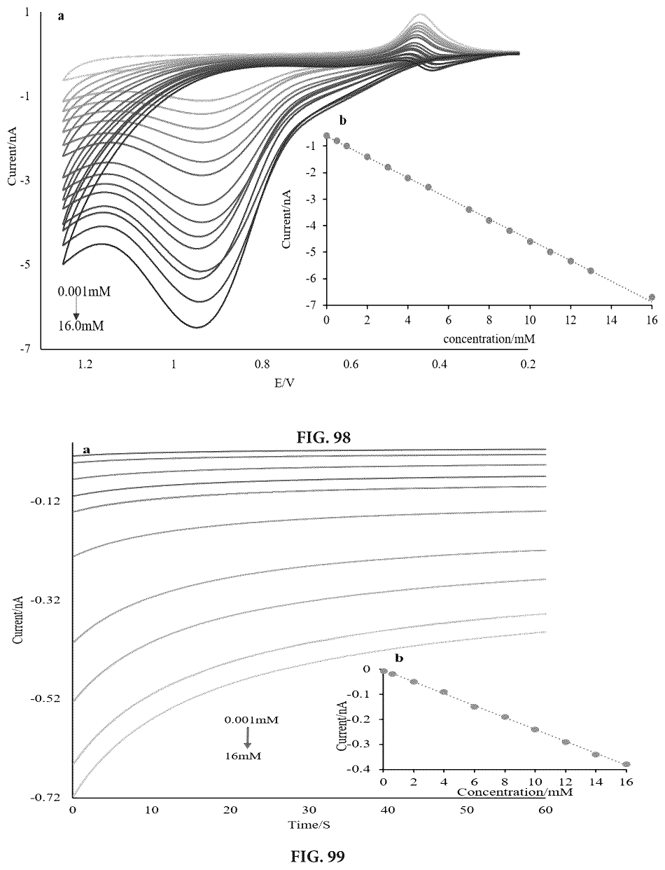

Furthermore, CV was conducted between 0.2 and 1.25 V as a quick and easy tool to describe microbiosensor to detect glucose. FIG. 98 panel a shows the CVs using PANI-GOx-GNPs-PPMP/GME with the successive addition of glucose from 0.001-16.0 mM in a PBS (pH 7.0)/0.1 mM Mn (CH₃COO)₂ solution saturated with oxygen gas at scan rate 0.05 Vs⁻¹. As shown in FIG. 98 panel a, the anodic peak current increased linearly at 0.94V and is accompanied by a decrease of the reduction peak at 0.44 V on addition of glucose (R²=0.999) with a LOD of 0.3 μM (S/N=3) (FIG. 98 panel b). The significant increasing of the anodic peak indicates that the immobilized PPMP enzyme was redox-active on the microelectrode toward the oxidation of the glucose.

An extremely attractive feature of the of PANI-GOx-GNPs-PPMP/GME electrode is its highly stable amperometric response toward glucose oxidation. CA can provide a more direct way to characterize sensitivity and selectivity compared to LSV and CV. The amperometric response of the PANI-GNPs-GOx-PPMP/GME biosensor was carried out under a similar cell setup. The steady-state current for oxidation of glucose was examined at different applied potentials of 0.60 to 0.90 V and at the 0.75 V, a better response was observed with a faster response time compared to other potentials. With the further increase of glucose concentration over the range of 0.001 to 16.0 mM the oxidation current of chronoamperometric curve trended towards a constant value within 40 seconds (FIG. 99 panel a). The microelectrode shows excellent sensitivity with an LOD of 0.5 μM (FIG. 99 panel b). It demonstrates a stable and an excellent electrocatalytic property of the immobilized enzyme on the PANI-GNPs-GOx-PPMP/GME. The LOD for PANI-GOx-GNPs-PPMP/GME electrode comparably is lower than GOx/Au/MXene/Nafion/GCE (Huangxian et al., 1998), GOD/poly-PDA (Komathi et al., 2017, (PANI/MWCNTs/GOx/HRP (Lovi'c et al., 2017), and Au-Cys-GA-GOx (Rakhi et al., 2016) Error! Reference source not found. In addition, PANI-GOx-GNPs-PPMP/GME electrode has other distinct advantages such as quick preparation, simpler in construction and lower in cost.

3.2 Selectivity, Reproducibility and Stability of PANI-GNPs-Gox-PPMP/GME

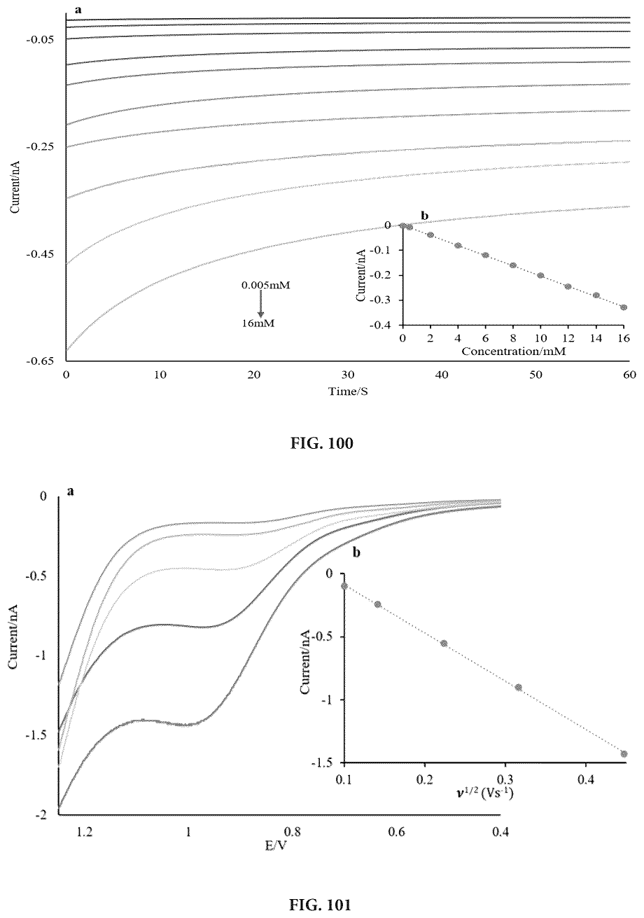

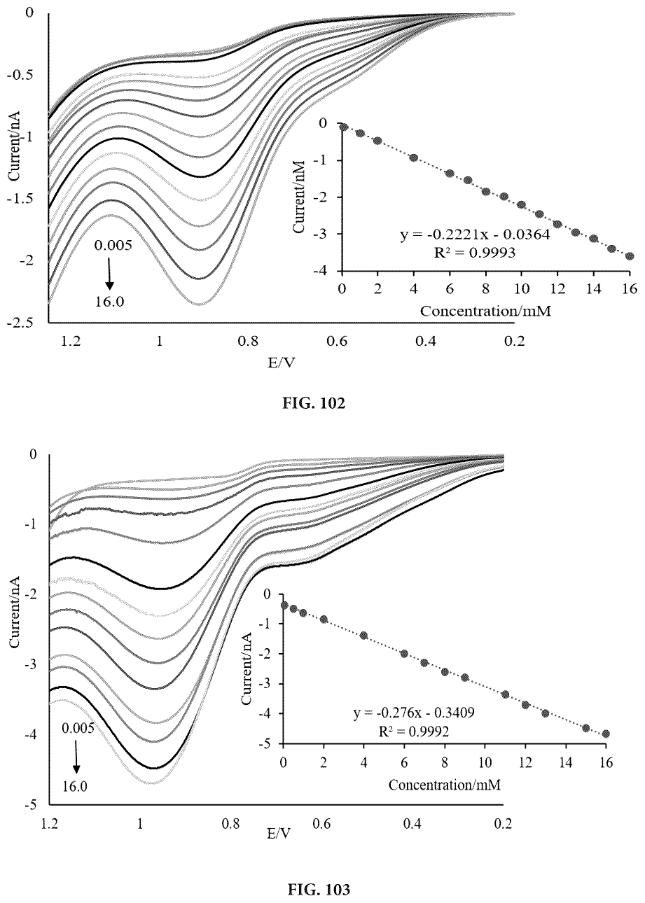

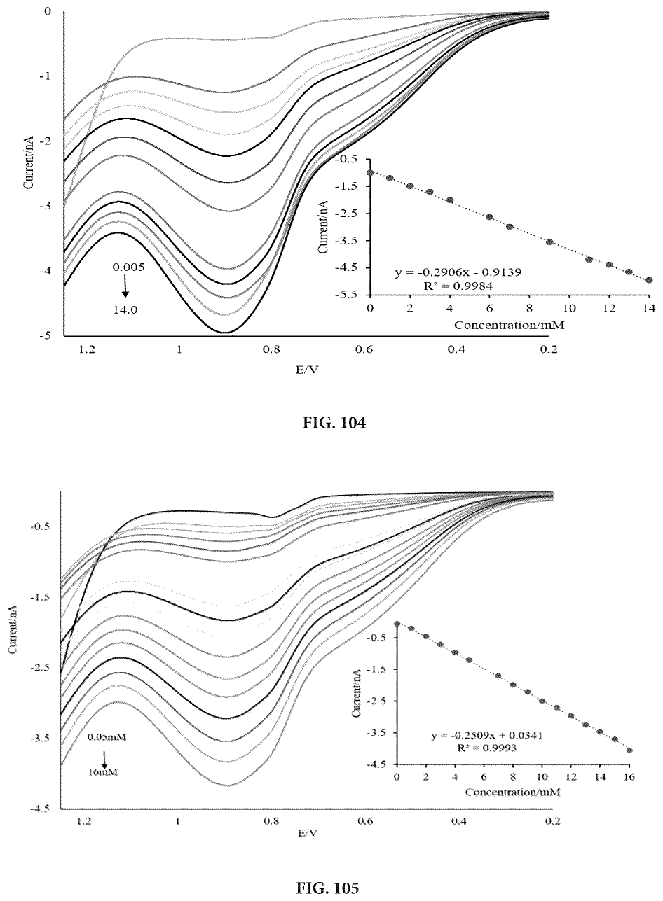

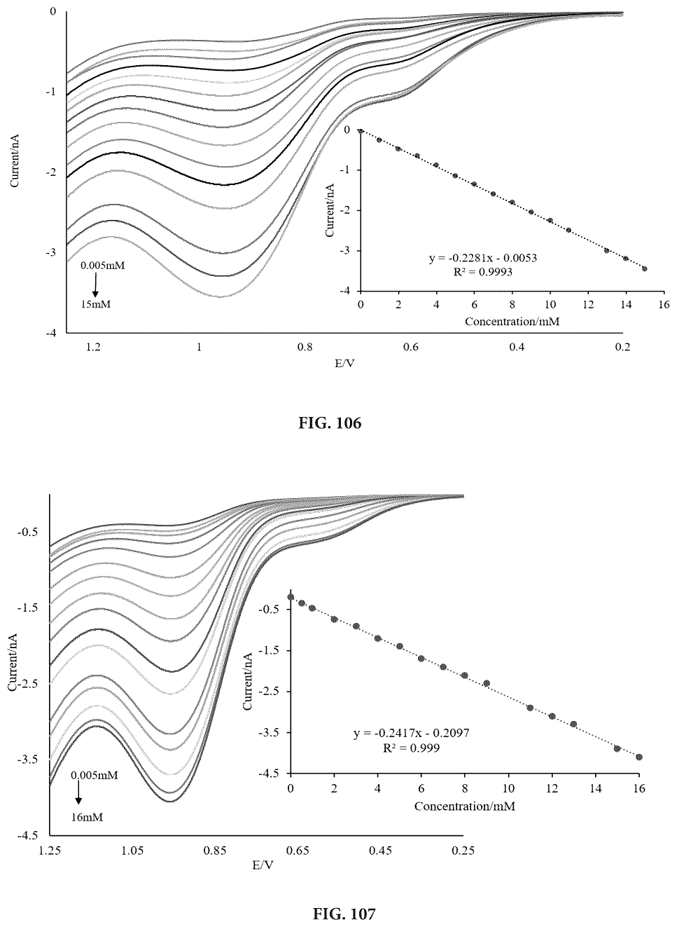

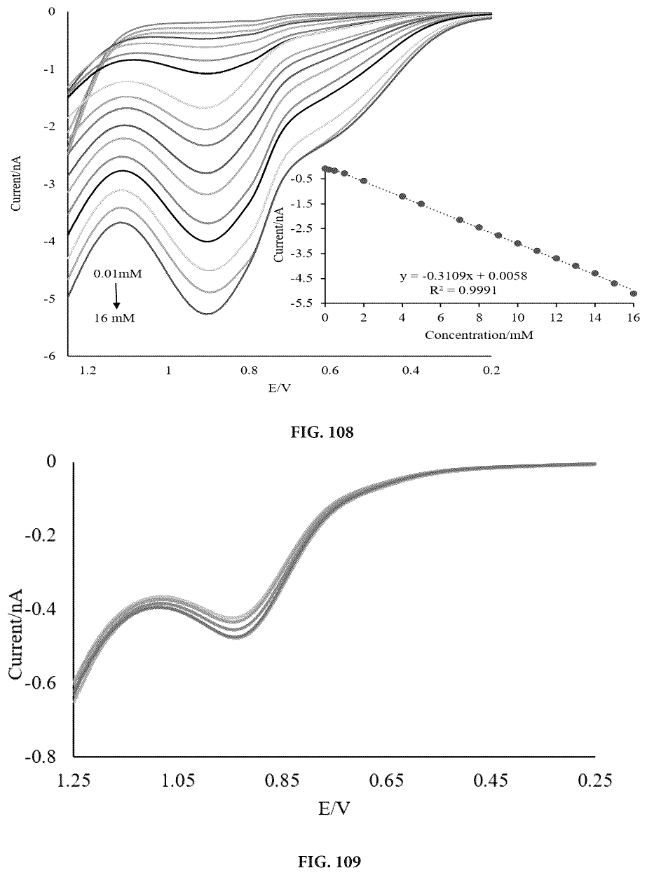

Selectivity is the most important feature of a sensor since high selectivity ensures excellent accuracy. A number of common materials were studied for any interfering effects in glucose analysis. The anti-interference ability of PANI-GNPs-GOx-PPMP/GME was investigated by the LSV technique. Known concentrations of AA, DA, aspartame, fructose, UA, caffeine, and 10% diet coke were tested as possible interfering compounds (FIGS. 102-108). Table 5 shows the response of a linear range of glucose concentration and an LOD in 1 mM constant concentration of interfering compounds using LSV technique. AA was also chosen to investigate using CA response on the PANI-GNPs-GOx-PPMP/GME under the same cell set up conditions at the potential of 0.75V. The response curves reached steady state after 40 s at various concentration ranges of glucose from 0.002 to 16.0 mM at 1.0 mM of AA, with an LOD of 1.0 μM and R² of 0.9990 (FIG. 100 panels a, b). No significant interfering effects were observed at 1 mM of these compounds on the analysis of glucose using CA and LSV methods. The experiments indicated only a minor difference in the responses and these results showed selectivity of PANI-GNPs-GOx-PPMP/GME, therefore, this preparation method for a glucose biosensor is favorable for selective monitoring of glucose concentrations. These results indicate that the PANI-GNPs-GOx-PPMP/GME can also undergo the catalytic oxidation of glucose in the presence of interfering chemicals.

TABLE 5
Influence of electroactive interferents on the response
of the microbiosensor using LSV method.

| | Adding | Concentration | |
|---|---|---|---|
| Interferent | content (mM) | range (mM) | LOD(μM) |
| Ascorbic acid | 1.0 mM | 0.005-16.0 | 1.5 |
| Caffeine | 1.0 mM | 0.005-16.0 | 1.7 |
| Dopamine | 1.0 mM | 0.005-14.0 | 2.0 |
| Fructose | 1.0 mM | 0.005-16.0 | 1.4 |
| Uric acid | 1.0 mM | 0.005-15.0 | 1.6 |
| Aspartame | 1.0 mM | 0.005-16.0 | 1.5 |
| Diet coke | 10% | 0.005-15.0 | 1.6 |

Repeatability of the modified microbiosensor was tested after seven times repeat measurements using the same electrode in 0.5 mM glucose in a single day using LSV (FIG. 109). It shows excellent repeatability with the relative standard deviation (RSD) value of 4.37% and the fabrication procedure was reproducible.

The PANI-GNPs-GOx-PPMP/GMEs were examined for their storage and operational stability by applying LSV and CA techniques. After the as-prepared microelectrodes were stored at 4° C. for 2 months, the current response was found to remain stable, retaining 95% of the initial current response. The results strongly support the long-term stability of the electrode and disturbances cannot cause drift in the output signals of biosensor response.

3.3 Different Scan Rate Responses of the PANI-GNPs-GOx-PPMP/GME

Furthermore, the diffusion-controlled electrochemical behavior of the PANI-GNPs-GOx-PPMP/GME was studied in 0.5 mM glucose in PBS pH 7/0.1 mM Mn(CH₃COO)₂ in an oxygen saturated solution. Different scan rate responses of PANI-GNPs-GOx-PPMP/GME in the potential windows between 0.2 and 1.25 V by varying different scan rates from 0.01 to 0.20 Vs⁻¹ were employed to assess the dynamics of heterogeneous electron transfer across the electrode/film layer interface. As FIG. 101 panel a shows the peak current (iₚ) increases at faster voltage scan rates. By increasing the voltage, the concentration of glucose at the microelectrode surface also alters, so a faster sweep of voltage causes a greater concentration gradient near the microbiosensor, making a larger peak current. The oxidation peaks current increased linearly with the square root of the scan rate with R² of 0.9994 (FIG. 101 panel b), indicating a diffusion-controlled process, according to the Randles Sevcik equation (Bard (2001)):

$$\\ {i}_{p} = ( 2.687 \times {10}^{5} ) {n}^{\frac{3}{2}} A \cdot {C ( D \cdot v )}^{1 / 2}$$

where peak current (iₚ) depends on the concentration (C), the diffusion coefficient (D), n is the number of electrons transferred, v is the scan rate in Vs⁻¹, and the electrode surface area (A). The results show the establishment of the electrode potential against the diffusion of the glucose concentration gradient, indicating that the process was controlled by diffusion and the electron transfer processes involve freely diffusing glucose.

Conclusions

In summary, PANI-GNPs-GOX-PPMP/GME indicates significantly reduced charge transfer resistance while we minimized the size of the target electrode to 10 μm. The results also demonstrated that electrodeposition of GOx-PPMP along with GNPs onto GME is an effective electrochemical technique for improving sensitivity and the electron transfer rate to detect glucose.

Through the electrochemical responses using CA. CV, and LSV as analytical techniques, the PANI-GNPs-GOx-PPMP/GME exhibits a wide linear range from 0.001 to 16.0 mM with a detection limit of 0.5 μM, 0.001 mM to 16.0 mM with a detection limit of 0.3 μM and from 0.002 to 16.0 mM with a detection limit of 0.6 μM respectively.

Additionally, this biosensor allows the determination of glucose in the presence of AA, DA, aspartame, fructose, UA, caffeine, and 10% diet coke.

The demonstrated microbiosensor is low-cost, robust, highly sensitive, selective, reproducible, and stable with a facile immobilization procedure.

Thus the biosensor possessed good stability and is a promising analytical tool for medical use and detecting glucose in food products and mass production.

Abbreviations: AA—ascorbic acid, CA—chronoamperometric, CV—cyclic voltammetry, DA—dopamine. GNPs—gold nanoparticles, GM—gold microelectrode, GME—gold microelectrode, GOx—glucose oxidase, HRP—Horseradish peroxidase, LOD—lower detection limit, LSV—Linear Sweep Voltammetry, PANI—polyaniline, PBS—Phosphate buffer solution, PPMP—plant-produced manganese peroxidase from corn, RSD—relative standard deviations, UA—uric acid;

### REFERENCES CITED IN THIS EXAMPLE

Bard A. J., Faulkner, L. R., White, H. S., 2022. Electrochemical Methods: Fundamentals and Applications; John Wiley & Sons, 3rd edn. New York, 2022.
Bucur, B., Purcarea, C., Andreescu, S., Vasilescu, A., 2021. Addressing the selectivity of enzyme biosensors: solutions and perspectives, review, Sensors 21, 3038-3074, https://doi.org/10.3390/s21093038
Fenoy, G. E., Giussi, J. M., Bilderling, C. V., Maza, E. M., Pietrasanta, L. I., Knoll, W., Marmisollé, W. A., Azzaroni, O., 2018. Reversible modulation of the redox activity in conducting polymer nanofilms induced by hydrophobic collapse of a surface-grafted polyelectrolyte. J. Colloid Interface Sci 518, 92-101, https://doi.org/10.1016/j.jcis.2018.02.014.
Gifford, R., 2013. Continuous glucose monitoring: 40 years, what we've learned and what's next. ChemPhysChem. 14, 2032-2044, https://doi.org/10.1002/cphc.201300172.
Guzinski, M., Jarvis, J. M., D'Orazio, P., Izadyar, A., Pendley, B. D., Lindner, E., 2017. Solid contact ph sensor without co2 interference with a super hydrophobic pedot-c14 as solid contact: the ultimate “Water Layer” Test“. Anal. Chem. 89, 8468-8475, https://doi.org/10.1021/acs.analchem.7b02009.
Harris, D. C., 2020. Quantitative Chemical Analysis, 10th ed.; W. H. Freeman and Company: New York, 2020.
Huangxian, J., Dongmei, Z., Yi, X., Hongyuan, C., 1998. Amperometric biosensor for glucose based on a nanometer-sized microb and gold electrode coimmobilized with glucose oxidase and poly(o-phenylenediamide), Electroanalysis 10, 541-545, https://doi.org/10.1002/(SICI)1521-4109(199807).
Izadyar, A., 2018. Stripping voltammetry at the interface between two review, immiscible electrolyte solutions: a Electroanalysis 30, 2210-2221, https://doi.org/10.1002/elan.201800279.
Izadyar, A., Al-Amoody, F., Ranawaka Arachchige, D., 2016. Ion transfer stripping voltammetry to detect Nanomolar concentrations of Cr (VI) in drinking water, J. Electroanal. Chem. 782, 43-49, https://doi.org/10.1016/j.jelechem.2016.10.002
Izadyar, A., Hershberger, J. C., Robert, R. 2018. Voltammetric assessment of ions transfer at ionophore-graphene based polymeric membranes, Electroanalysis 30, 1-5, https://doi.org/10.1002/elan.201800535.
Izadyar, A., Ranawaka Arachchige, D., Cornwell, H., Hershberger, J. L., 2016. Ion transfer stripping voltammetry for the detection of nanomolar levels of fluoxetine, citalopram, and sertraline in tap and river water samples, Sens. Actuators B: Chem. 223, 226-233, https://doi.org/10.1016/j.snb.2015.09.048.
Izadyar, A., Rodriguez, K. A., Van, M. N., Tran, U., Hood, E. E., 2021. A bienzymatic amperometric glucose biosensor based on using a novel recombinant Mn peroxidase from corn and glucose oxidase with a Nafion membrane, J. Electroanal. Chem. 895,115387. https://doi.org/10.1016/j.jelechem.2021.115387
Izadyar, A., Tran, U., Ilwoo, S., Hood, E. E., 2019. Recombinant Mn peroxidase from corn grain has an excellent electrocatalytic effect in a designed amperometric biosensor to detect hydrogen peroxide at low concentrations, ACS Sustainable Chem. Eng. 7, 19434-19441. https://doi.org/10.1021/acssuschemeng.9b04216.
Jarvinen, J., Taskila, S., Isomaki, R., Ojamo, H., 2012. Screening of white-rot fungi Manganese peroxidases: a comparison between the specific activities of the enzyme from different native producers, AMB Express 2, 62. http://www.amb-express.com/content/2/1/62.
Kaur, H., Shorie, M., 2019 Nanomaterial based aptasensors for clinical and environmental diagnostic applications, Nanoscale Adv. 1, 2123-2138. https://doi.org/10.1039/C9NA00153K.
Komathi, S., Gopalan, A. I., Muthuchamy, N., Lee, K. P., 2017. Polyaniline nanoflowers grafted onto nanodiamonds via a soft template-guided secondary nucleation process for high-performance glucose sensing. RSC Adv., 7, 15342-15351. DOI: doi.org/10.1039/C6RA24760A.
Kozai, T. D. Y., Jaquins-Gerstl, A. S., Vazquez, A. L., Michael, A. C., Cui, X. T., 2015. Brain tissue responses to neural implants impact signal sensitivity and intervention strategies, ACS Chem. Neurosci. 6, 48-67. https://doi.org/10.1021/cn500256e.
Lovic, J., Stevanovic, S., Nikolic, N. D., Petrovic, S., Vukovic, D., Prlainovic, N., Mijin, D., Avramov Ivic, M., 2017. Glucose sensing using glucose oxidase-glutaraldehyde-cysteine modified gold electrode, Int. J. Electrochem. Sci. 12, 5806-5817. https://doi.org/10.20964/2017.07.65.
Marinesco, S., Frey, O. 2013. Microelectrode Biosensors, Springer, https://doi.org/10.1007/978-1-62703-370-1, 2013.
Olczuk, D., Priefer, R. A., 2018. A history of continuous glucose monitors (cgms) in self-monitoring, Diabetes Metab. Syndr, Clin. Res. Rev. 12, 181-187. https://doi.org/10.1016/j.dsx.2017.09.005.
Pavel, I. A., Lakard, S., Lakard, B., 2022. Flexible sensors based on conductive polymers, review: Chemosensors 10, 97. doi.org/10.3390/chemosensors10030097.
Rakhi, R. Nayak, B. P., Xia, C., Alshareef, H. N., 2016. Novel amperometric glucose biosensor based on MXene nanocomposite, Sci Rep. 6,36422. https://doi.org/10.1038/srep36422.
Rivnay, J., Inal, S., Collins, B. A., Sessolo, M., Stavrinidou, E., Strakosas, X., Tassone, C., Delongchamp, D. M., Malliaras, G. G., 2016. Structural control of mixed ionic and electronic transport in conducting polymers, Nat. Commun. 7,11287-11296. https://doi.org/10.1038/ncomms11287.
Senf, B., Yeo, W. H., Kim, J. H., 2020. Recent advances in portable biosensors for biomarker detection in body fluids, review; Biosensors 10,127. https://doi.org/10.3390/bios10090127.
Si, P., Razmi, N., Nur, O., Solanki, S., Pandey, C. M., Gupta, R. K., Malhotra, B. D., Willander, M., Zerda, A. de la., 2021. Gold nanomaterials for optical biosensing and bioimaging, Nanoscale Adv. 3, 2679-2698. https://doi.org/10.1039/D0NA00961J.
Simon, D. T., Gabrielsson, E. O., Tybrandt, K., Berggren, M., 2016. Organic bioelectronics: bridging the signaling gap between biology and technology, Chem. Rev. 116, 13009-13041. https://doi.org/10.1021/acs.chemrev.6b00146.
Tan, C., Robbins, E. M., Wu, B., Cui, X. T., 2021. Recent advances in in vivo neurochemical monitoring, Micromachines. 12, 208-240. doi.org/10.3390/mi12020208.
Varnakavi, N., Nohyun, L., 2021. A review on biosensors and recent development of nanostructured materials-enabled biosensors, Sensors 21,1109-1143. https://doi.org/10.3390/s21041109.
Wilson, M. S., 2005. Electrochemical immunosensors for the simultaneous detection of two markers, tumor Anal. Chem. 77, 1496-1502. https://doi.org/10.1021/ac0485278.
Woeppel, K. M., Zheng, X. S., Schulte, Z. M., Rosi, N. L., Cui, X. T., 2019. Nanoparticle doped PEDOT for enhanced electrode coatings and drug delivery, Adv. Healthc. Mater. 8, 1900622. https://doi: 10.1002/adhm.201900622.

### Example 16

Using Corn to Develop a Disposable Electrochemical Sensor for Glucose Detection.

Diabetes is a chronic disease that affects millions of individuals worldwide and ranks as a leading cause of death and disability (1, 2). Monitoring blood glucose levels is crucial in diagnosing diabetes, as glucose stands as the most frequently analyzed analyte. Electrochemical devices can meet this need, including the production of flexible biosensors (3-8). The standard set by the World Health Organization (WHO) establishes the fasting blood glucose (FBG) level for normal individuals in the range of 3.9-6.1 mM, with postprandial blood glucose ideally being 7.8 mM or lower. Individuals exhibiting typical diabetes symptoms are diagnosed with diabetes if their random blood glucose level is ≥11.1 mM, FBG is ≥7.0 mM, or postprandial blood glucose level is ≥11.1 mM. An often-utilized approach in bi-enzymatic sensors involves utilizing glucose oxidase (GOx) and horseradish peroxidase (HRP) to detect glucose levels. However, HRP comes with several disadvantages due to its high cost, limited reaction conditions, and low catalytic efficiency in reducing hydrogen peroxide (H₂O₂). Findings from our research highlight the robust activity of plant produced manganese peroxidase (PPMP) across this entire range (9-12). In the realm of electrochemical sensing, screen-printed electrodes (SPEs) have gained significant popularity due to their versatility, ease of fabrication, potential for miniaturization, portability, low sample volume requirements, rapid response times, reduced background noise, compatibility with various detection methods, disposability, robustness, excellent detection limits, selectivity, sensitivity, and cost-effectiveness, combined with the ability for mass production (13-17). The utilization of a new recombinant enzyme derived from corn (PPMP) in conjunction with horseradish peroxidase (HRP) and screen-printed electrodes for electrochemical sensing presents a breakthrough for a stable, cost-effective, environmentally friendly, and disposable glucose sensor. By using 50 mg of PPMP, we can produce 200 disposable enzyme-based sensors, which can provide economic benefits. The Phase I describes non-limiting, exemplary tests to investigate the accuracy, safety, and usability of the sensor.

Development of a screen-printed electrode (SPE) along with enzyme PPMP tailored for the selective diagnosis of glucose using advanced electrochemical techniques.

This effort involves the integration of Square Wave Voltammetry (SWV) (18) into the modified PPMP/SPE membrane.
To achieve this, we can implement modifications to both the PPMP (Recombinant Mn Peroxidase from Corn) and the membrane nanocomposite, which includes nanoparticles and conductive polymers.
Apply antifouling coatings on the surface of the transducer to minimize nonspecific binding of biomolecules.
These modifications can enhance the dynamic range and sensitivity of the electrode system.

### Testing the Modified Screen-Printed Electrode (SPE) for Glucose Detection.

Testing glucose monitoring accuracy.
Controlling membrane permeability.
Minimizing interference from other substances.
Testing user interface and accessibility.
Ensuring stability and long-term monitoring.

The success achieved in Phase I can serve as a foundation for testing during Phase II. This phase will encompass pre-clinical studies. Moreover, Phase II will involve a comprehensive investigation into the characteristics of the expanded assay using clinical samples. Without wishing to be bound by theory, we describe the development of the first cheap, disposable glucose monitoring sensor using corn-based technology. This advancement can revolutionize glucose monitoring and enhance patient care.

Research Strategy

More than 90% of individuals with diabetes have type 2, and the International Diabetes Federation predicts a significant increase in the proportion of the global population affected by diabetes, resulting in a continuous rise in the number of people with the condition (19). The World Health Organization (WHO) reports approximately 450 million cases of diabetes worldwide, a number that can reach 700 million by 2045. Projections indicate a rise to 39.7 million by 2030 and 60.6 million in 2060 in the United States alone (20). Therefore, it is important for inexpensive and stable technologies to be developed to monitor glucose. Electrochemical biosensors, functioning as biological devices, are becoming increasingly prevalent and integral to medical, industrial, and environmental applications. The most frequently employed enzymes in most of the available biosensors are glucose oxidase (GOx) and horseradish peroxidase (HRP) (21). In contrast, manganese peroxidase (MP) is acknowledged as a potent oxidizing enzyme, showing promising potential in various applications (22, 24) (Table 6). We have introduced a new enzymatic biosensor utilizing recombinant plant-produced MP (PPMP). Our study indicates that this engineered manganese peroxidase (PPMP) displays activity in detecting H₂O₂ and glucose (Table 7) (13-17). The research findings indicate that the detected glucose range aligns with the acceptable standards established by the World Health Organization (WHO). To address the cost factor, we describe an economical and reliable disposable sensor using SPE. Additionally, we incorporate conductive polymers like polyaniline, polypyrrole, or polythiophene, to act as mediators for electron transfer within screen-printed glucose sensors. A non-limiting, exemplary noteworthy breakthrough in this endeavor is the utilization of PPMP, enabling the creation of electrochemical biosensors with heightened selectivity and sensitivity for detecting low glucose concentrations. Through this integration, we can enhance accuracy, sensitivity, stability, and overall biosensor performance. (25-27).

TABLE 6
Comparing PPMP (Recombinant Mn Peroxidase from
Corn) and HRP (Horseradish Peroxidase) (24).

| | MnP from corn | |
|---|---|---|
| Characteristic | (PPMP) | HRP |
| MW | 53,000 Da | 43,000 Da |
| # isozymes | 1 | 7 |
| Enzyme # | 1.11.1.13 | 1.11.1.7 |
| Substrate | H₂O₂, Mn | H₂O₂ |
| pH optimum | 5-7 | 7 |
| Concentration in | 5 g/kg corn kernels | 156 mg/kg |
| biomass | | (horseradish roots |
| | | “Amoracia Rusticana”) |
| Purified cost | $50-$75/g* | $1250/g* |
| Source | Infinite Enzymes, | BBI Solutions, Sigma |
| | LLC | Chemical Co. |

TABLE 7
the various sensors we fabricated using PPMP. The results
cover a range of concentrations and detection limits.
PANI (polyaniline), BSA (bovine serum albumin), AuNPs
(gold nanoparticles), GME (gold microelectrode) (9-12).

| | | | Linear | LOD |
|---|---|---|---|---|
| No. | Modified electrodes | Eₚ^{C}(V) | range (mM) | (mM) |
| 1 | Nafion/PPMP-GOx- | 0.95 ± 0.03 | 0.1-15.0 | 0.019 |
| | BSA/Au | | | |
| 2 | PANI- GOX- PPMP- | 0.55 ± 0.06 | 0.025-5.5 | 0.009 |
| | BSA/Au | | | |
| 3 | PANI- AuNPs- GOX- | 0.79 ± 0.04 | 0.005-6.0 | 0.0016 |
| | PPMP-BSA/Au | | | |
| 4 | PANI- GOX- PPMP- | 0.45 ± 0.04 | 0.015-6.0 | 0.005 |
| | BSA/AuNPs/Au | | | |
| 5 | PANI- AuNPs- GOX- | 0.90 ± 0.05 | 0.001-16.0 | 0.0005 |
| | PPMP-BSA/GME | | | |

Reports indicate that the market size for diabetes devices was valued at USD 27,662.11 million in 2021 and is projected to reach USD 49,253.08 million by 2030. North America and Europe are the two most significant markets for diabetes devices, with North America expected to be the largest market, valued at USD 18,943.58 million by 2030. These expanding market sizes and technological advancements are positive indicators of progress in diabetes management, offering hope for a brighter future for individuals living with diabetes and healthcare providers alike.

### Non-Limiting, Exemplary Innovative Aspects

The integration of screen-printed electrodes, nanoparticles, Recombinant Mn Peroxidase from Corn (PPMP), glucose oxidase, conductive polymers, and the utilization of Square Wave Voltammetry (SWV) presents a comprehensive solution to numerous challenges within glucose sensor technology. This approach addresses the following areas of innovation:
Sensitivity and Selectivity: Improving the ability to detect glucose accurately and selectively.
Stability: Ensuring consistent performance over time and various conditions.
Cost-Effectiveness and Accessibility: Creating an affordable solution that is accessible to a wider population.
Miniaturization: Designing compact and wearable sensors for convenience.
Longevity and Reduced Drift: Extending the sensor's lifespan while minimizing measurement errors.
Improved Signal Detection: Enhancing the detection and interpretation of glucose levels.
Closed-Loop Integration: Enabling real-time adjustments and automated glucose management.

This integrated approach represents a significant step forward in overcoming the challenges associated with glucose sensor technology.

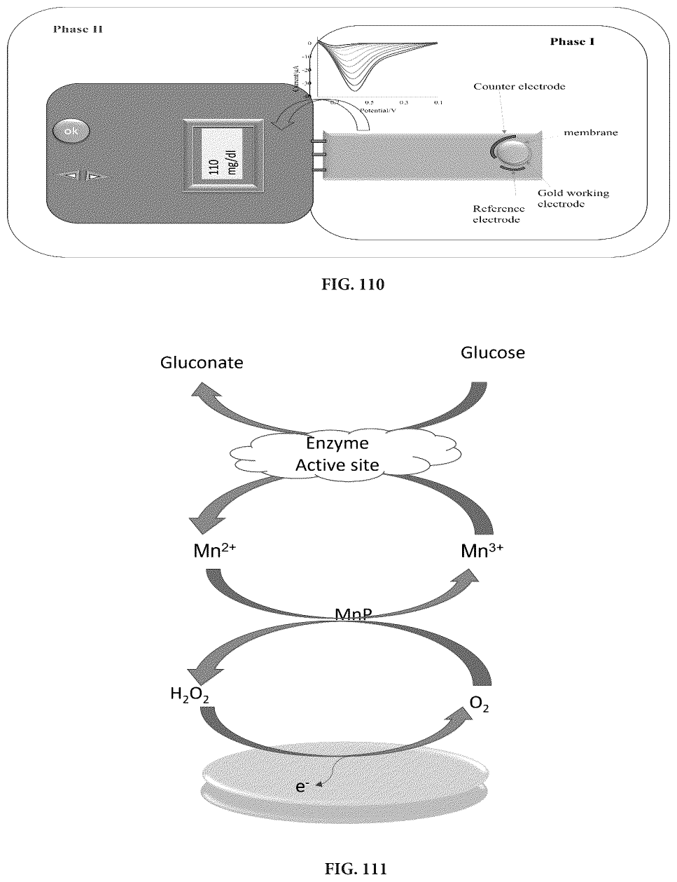

FIG. 110 illustrates the future application of the enzyme-based glucose sensor, integrated into a portable device using screen-printed electrodes (SPE), for direct glucose level measurement. This technology holds immense promise in advancing glucose sensor innovation and improving the quality of life for individuals managing diabetes or related conditions. The electropolymerization of the composite directly onto the screen-printed electrode in a single step represents a significant breakthrough in glucose sensor development. This approach offers several key benefits and advantages, including simplicity and convenience, stability and adhesion, homogeneous film formation, increased sensitivity, miniaturization and portability, selectivity and specificity, and low sample volume requirements. Our vision for this integration exemplifies innovation at its finest, as it directly addresses real-world challenges and offers tangible benefits for patients managing diabetes. This advancement has the potential to revolutionize diabetes care and marks a substantial leap forward in the field of glucose monitoring technology.

Non-Limiting, Exemplary Studies: The collection and analysis of non-limiting, exemplary data from these tests can allow for the evaluation of sensor performance in comparison to existing glucose sensors or reference methods. Collaboration with clinicians and diabetes specialists plays a role in the research process. Involving experts from the medical field ensures that the sensor's design and functionality align with the practical needs and requirements of real healthcare settings. The promising outcome of this collaboration is the potential to bring this innovative technology to the market, ultimately benefiting individuals living with diabetes.

Non-Limiting Electrodeposition Procedure: We used an electrochemical method to clean the Screen-Printed Electrodes (SPEs) by performing cyclic voltammetry in two different solutions: 1.0 M H₂SO₄ and PBS (Phosphate Buffer Solution). For the cleaning process in 1.0 M H₂SO₄, we swept the potential between −0.1 to +1.0 V for five cycles at a scan rate of 0.05 V/s. This helped remove any contaminants or impurities from the electrode surface, ensuring a clean starting point for further modifications. We also cleaned the electrode using the same conditions but in PBS to ensure the removal of any remaining traces of unwanted substances. After the cleaning steps, we dried the electrodes using nitrogen gas and deposited 6 μL of the composite onto the working electrode (WE) surface. The composite consisted of PPMP. GOX (glucose oxidase). GNPs (gold nanoparticles), and CPs (conductive polymers). Electropolymerization was then carried out on the SPEs by sweeping the potential between −0.10 to +1.6 V at a scan rate of 0.05 V/s for 30 to 15 cycles. Our study found that the nanocomposite film showed the best stability when electropolymerized for 25 cycles. To preserve the stability and performance of the modified electrodes, we stored them in an incubator at a temperature of four ° C. when not in use.

Apparatus: We utilized a computer controlled CHI660D electrochemical workstation (CH Instruments. Austin, TX) to conduct all electrochemical measurements. The experiments were conducted using disposable/reusable screen-printed electrodes, which comprised a 3 mm gold working electrode (WE), a silver reference electrode (RE), and a carbon counter electrode (CE) (FIG. 110).

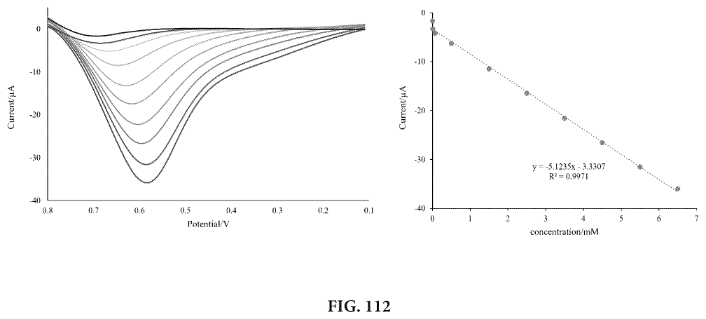

The catalytic effect of enzymes on Glucose detection. FIG. 111 illustrates electron transfer and glucose detection. In the catalytic process depicted in the figure, the glucose oxidase (GOx) reaction generates hydrogen peroxide (H₂O₂). This produced hydrogen peroxide, in the presence of glucose, serves as a substrate for manganese peroxidase (MnP). MnP utilizes hydrogen peroxide and a reducing agent (usually a low-molecular-weight compound) to oxidize Mn (II) to Mn (III) at its heme active site. The presence of Mn (III) at the enzyme's active site enables it to effectively catalyze the oxidation of glucose to gluconolactone. The overall reaction can be summarized as follows:

Glucose+O₂→Gluconolactone+H₂O₂(by GOx)

Mn(II)+H₂O₂→Mn(III)+H₂O(by MnP)+1/2O₂

The catalytic cycle of these enzymes assists in the detection and quantification of glucose by engaging in redox reactions that involve glucose, molecular oxygen, hydrogen peroxide, and manganese ions.

Square Wave Voltammetry (SWV) sensing of PANI-GNPs-GOx-PPMP/SPE. FIG. 112 panel B illustrates the calibration curves constructed from the data obtained through SWV measurements. The x-axis represents the concentration of glucose in mM (millimoles per liter), while the y-axis displays the corresponding current response in microamps (μA). These calibration curves demonstrate a linear relationship between glucose concentration and current response. This linearity indicates that the sensor's response to glucose is directly proportional to its concentration within the tested range. The slope of the calibration curve signifies the sensor's sensitivity, representing the change in current per unit change in glucose concentration. These calibration curves serve the purpose of quantification. When the sensor encounters an unknown glucose concentration, the corresponding current response acquired from the SWV measurement enables the determination of glucose concentration based on the calibration curve. The distinctive peak-shaped voltammograms observed in FIG. 112 panel A highlight the sensor's efficacy in detecting glucose at a specific potential (0.58 V). This peak corresponds to the electrochemical oxidation of glucose, where glucose molecules are oxidized at the sensor's surface, resulting in the generation of a measurable current. Overall, the optimization of the SWV parameters and the construction of calibration curves demonstrate the successful development and validation of the PPMP enzyme-based glucose disposable sensor. Further validation and testing in diverse samples and real-world scenarios will be essential to establish the sensor's robustness and suitability for clinical applications. The limit of detection (LOD) for glucose, as mentioned in the previous discussion, was reported to be 0.044 mM. This LOD value represents the minimum concentration of glucose that the CPs-GOx-GNPs-PPMP/SPE sensor can reliably detect.

Enhancing Glucose Diagnosis Through Advanced Electrochemical Techniques

A specialized screen-printed electrode (SPE) in tandem with the tailored enzyme PPMP is described herein. This strategic enzyme integration can allow for targeted glucose diagnosis, leveraging sophisticated electrochemical methods.

To enhance the precision and credibility of glucose measurements, we incorporate Square Wave Voltammetry (SWV) alongside a modified Screen-Printed Electrodes (SPEs) membrane. Key factors are meticulously addressed to ensure optimal outcomes:

Modifying PPMP and Membrane Nanocomposite: Optimizing biosensors for glucose monitoring involves intricate electrochemical alterations to both PPMP enzyme and the membrane nanocomposite. This entails integrating nanoparticles and conductive polymers to augment system performance.

Application of antifouling coatings to the transducer surface to minimize nonspecific biomolecule binding.

These enhancements can broaden the dynamic range and sensitivity of the electrode system.

Exploration of diverse nanoparticles like gold, carbon nanotubes, and graphene to amplify electrode surface area.

Investigation of conductive polymers such as polypyrrole and polyaniline to bolster electrode conductivity and foster a microenvironment for enzyme immobilization.

Employing the electropolymerization technique for enzyme immobilization, a method with benefits in biosensor development, for example in glucose monitoring.

Dynamic Range and Sensitivity: Ensuring the sensor's capacity to detect a comprehensive array of glucose concentrations, encompassing both normal levels and heightened concentrations observed post glucose load during tolerance tests. Testing under varying conditions safeguards consistent and precise performance in real-world glucose tolerance tests.

### Non-Limiting, Exemplary Outcomes for Aim 1

Deliberate refinement of processes and component ratios within the sensing interface, culminating in heightened sensitivity and selectivity.

Identification of effective membrane composition combinations for the proposed glucose sensor, achieved through methodical experimentation and analysis.

Testing the Modified Screen-Printed Electrode (SPE) for Glucose Detection.

We can provide a process for the modified screen-printed electrode (SPE), designed for glucose detection. We can investigate:

Testing Glucose Monitoring Accuracy: Assessments are conducted to investigate the accuracy of glucose monitoring. We can achieve precise and consistent glucose measurements that align with established standards.

Control of Membrane Permeability: The permeability of the membrane is controlled and optimized to provide balance between glucose detection sensitivity and selectivity. This step can enhance the reliability of glucose measurements.

Minimization of Interference from Other Substances: Thorough experimentation and analysis are carried out to minimize interference from various substances that can impact glucose detection. We can enhance the sensor's specificity, enabling accurate glucose measurement in complex biological matrices.

Testing User Interface and Accessibility: The user interface is subjected to comprehensive testing to ensure user-friendliness and accessibility. The design and operation can a practical and effective glucose monitoring solution.

Ensuring Stability and Long-Term Monitoring: Extensive stability testing is undertaken on the sensor's performance over an extended duration. This step indicates the sensor's suitability for long-term glucose monitoring, an aspect in diabetes management.

By addressing these key elements, we can establish the reliability, accuracy, and practicality of our modified SPE for glucose detection. This aim represents a vital step in bringing our innovative glucose sensor technology closer to real-world application and impact.

### Aim 2: Non-Limiting, Exemplary Outcomes

Cutting-Edge Glucose Sensor: Without wishing to be bound by theory. Aim 2 can produce an innovative glucose SPE sensor using PPMP, showcasing exceptional performance in robustness, sensitivity, accuracy, safety, and usability—an instrumental leap in glucose monitoring technology.

### REFERENCES CITED IN THIS EXAMPLE

Seshasai, S. R., Kaptoge, S., Thompson, A., Angelantonio, E. D., AND Gao, P. (2011). Diabetes mellitus, fasting glucose, and risk of cause-specific death. N Engl J Med. 364, 829-41. doi: 10.1056/NEJMoa1008862.
Ghebreyesus, T. A. (2019). World report on vision. Geneva: World Health Organization, https://www.who.int/publications-detail-redirect/world-report-on-vision (accessed Jun. 29, 2020).
Pullano, S. A., Greco, M., Bianco, M. G., Foti, D., Brunetti, A., Antonino S., and Fiorillo, A. S. (2022). Review: Glucose biosensors in clinical practice: principles, limits and perspectives of currently used devices. Theranostics 12, 493-511. doi: 10.7150/thno.64035.
Battelino, T., and Bergenstal R. M. (2020). Continuous glucose monitoring-derived data report-simply a better management tool. Diabetes Care. 43: 2327-9, doi.org/10.2337/dci20-0032.
Fahmy Taha M. H., Ashraf, H., and Caesarendra, W. (2020). A Brief description of cyclic voltammetry transducer-based non-enzymatic glucose biosensor using synthesized graphene electrodes. Appl. Syst. Innov. 3: 32, doi.org/10.3390/asi3030032.
Johnston, L., Wang, G., Hu, K., Qian, C., and Liu, G. (2021). Advances in Biosensors for Continuous Glucose Monitoring Towards Wearables. Front. Bioeng. Biotechnol. 9: 733810, doi: 10.3389/fbioe.2021.733810.
Hayat, A., Haider, W., Rolland, M., and Marty, J. L. (2013). Electrochemical grafting of long spacer arms of hexamethyldiamine on a screen-printed carbon electrode surface: Application in target induced ochratoxin A electrochemical aptasensor. Analyst, 138: 2951-2957, doi.org/10.1039/C3AN00158J.
Krzyczmonik, P., Socha, E., and Skrzypek, S. (2018). Electrochemical Detection of Glucose in Beverage Samples Using Poly (3,4-ethylenedioxy thiophene)-Modified Electrodes with Immobilized Glucose Oxidase. Electrocatalysis, 9:380-387, doi.org/10.1007/s12678-017-0442-2.
Izadyar A., Van M. V., Miranda M., Weatherford S., Hood E. E., Electrocatalytic Effect of Recombinant Mn Peroxidase from Corn on Microbiosensors to Detect Glucose, Biocatalysis and Agricultural Biotechnology 43 (2022) 102445.
Izadyar A., Van M. V., Miranda M., Weatherford S., Hood E. E. (2022) Development of a Highly Sensitive Glucose Nanocomposite Biosensor Based on Recombinant Enzyme from Corn”, Journal of the Science of Food and Agriculture. 102: 6530-6538
Izadyar A., Rodriguez K. A., Van, M. N., Tran U., and Hood E. E. “A bienzymatic amperometric glucose biosensor based on using a novel recombinant Mn peroxidase from corn and glucose oxidase with a Nafion membrane”. Journal of Electroanalytical Chemistry 2021, 895, 115387.
Izadyar A., Tran U., Hood E. E. “Recombinant Mn Peroxidase from Corn Grain Has an Excellent Electrocatalytic Effect in a Designed Amperometric Biosensor to Detect Hydrogen Peroxide at Low Concentrations” ACS Sustainable Chem. Eng. 2019, 7, 19434-19441.
Hayat, A., Andreescu, S., and Marty, J. L. (2013). Design of PEG-aptamer two piece macromolecules as convenient and integrated sensing platform: application to the label free detection of small size molecules. Biosens. Bioelectron. 45:168-173, doi: 10.1016/j.bios.2013.01.059.
Pasakon, P., Mensing, J. P., Phokaratkul, D., Karuwan, C., Lomas, T., Wisitsoraat, A., and Tuantranont, A. (2019) A high-performance, disposable screen-printed carbon electrode modified with multi-walled carbon nanotubes/graphene for ultratrace level electrochemical sensors. J. Appl. Electrochem. 49:217-227, doi.org/10.1007/s10800-018-1268-1.
Couto, R. A. S., Lima, J. L. F. C., and Quinaz, M. B. (2016). Recent developments, characteristics and potential applications of screen-printed electrodes in pharmaceutical and biological analysis. Talanta. 146:801-814, doi.org/10.1016/j.talanta.2015.06.011.
Ping, J., Wu, J., Wang. Y., and Ying, Y. (2012) Simultaneous determination of ascorbic acid, dopamine and uric acid using high-performance screen-printed graphene electrode, Biosens. Bioelectron. 34:70-76, doi.org/10.1016/jbios.2012.01.016.
Yin, T., and Qin, W. (2013) Applications of Nanomaterials in Potentiometric Sensors. Trends Anal. Chem. 51:79-86, doi.org/10.1016/j.trac.2013.06.009.
A. J. Bard L. R. Faulkner, Electrochemical Methods: Fundamentals and Applications; John Wiley & Sons, 2nd edn. New York (2001).
Siddiqui, S. A.; Zhang. Y.; Lloret, J.; Song. H.; Obradovic, Z. Pain-Free Blood Glucose Monitoring Using Wearable Sensors: Recent Advancements and Future Prospects. IEEERev. Biomed. Eng. 2018, 11,21-35.
Villena Gonzales, W.; Mobashsher, A. T.; Abbosh, A. The Progress of Glucose Monitoring—A Review of Invasive to Minimally and Non-Invasive Techniques, Devices and Sensors. Sensors 2019, 19, 800.
Wu P, Cai Z, Chen J, Zhang H, Cai C. 2011. Electrochemical Measurement of the Flux of Hydrogen Peroxide Releasing from RAW 264.7 Macrophage Cells Based on Enzyme-Attapulgite Clay Nanohybrids. Biosens. Bioelectron. 26:4012-4017.
Singh R, Grigg J C, Qin W, Kadla J F, Murphy M E P, Lindsay D, Eltis L D. 2013. Improved Manganese-Oxidizing Activity of DypB, a Peroxidase from a Lignolytic Bacterium. ACS Chem. Biol. 8: 700-706.
Wang W, Xie Y, Wang Y, Du H, Xia C, Tian F. 2014. Glucose biosensor based on glucose oxidase immobilized on unhybridized titanium dioxide nanotube arrays. Microchim. Acta, 181: 1325-1331.
D R Walwyn, S M Huddy, E P Rybicki, Techno-Economic Analysis of Horseradish Peroxidase Production Using a Transient Expression System in Nicotiana benthamiana, Applied biochemistry and biotechnology 175, 841-854.
Duygu Büyüktas, a,b, Masoud Ghaanib, Cesare Roverab, Daniele Carullob, Richard T. Olssonc, Figen Korela, Stefano Farrisb,d, A screen-printed electrode modified with gold nanoparticles/cellulose nanocrystals for electrochemical detection of 4,4′-methylene diphenyl diamine. Heliyon. 9(2023) e15327
Pasakon, P., Mensing, J. P., Phokaratkul, D., Karuwan, C., Lomas, T., Wisitsoraat, A., and Tuantranont, A. (2019) A high-performance, disposable screen-printed carbon electrode modified with multi-walled carbon nanotubes/graphene for ultratrace level electrochemical sensors. J. Appl. Electrochem. 49:217-227, doi.org/10.1007/s10800-018-1268-1.
Gabriela Martins de Araújo, Milton Alexandre Cardoso, Lúcia Codognoto, Christopher M. A. Brett, Fábio R. Simões (2023). Screen-Printed Electrodes Based on Conductive Inks of Polyaniline/Graphene Hybrids and Their Application to Progesterone Detection. ECS Adv. 2, 016504.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An electrochemical sensor for the measurement of glucose concentration comprising one or more electrodes, a coating that surrounds the one or more electrodes, and one or more enzymes distributed within the coating, wherein the one or more enzymes comprises peroxidase, glucose oxidase, or a combination thereof, and wherein the one or more electrodes comprises a screen-printed electrode.

2. (canceled)

3. The electrochemical sensor of claim 1, wherein the peroxidase comprises manganese peroxidase.

4. The electrochemical sensor of claim 1, wherein the peroxidase is recombinantly produced.

5.-8. (canceled)

9. The electrochemical sensor of claim 1, wherein the coating comprises manganese peroxidase polythiophene, glucose oxidase, bovine serum albumin (BSA), and glutaraldehyde.

10. (canceled)

11. The electrochemical sensor of claim 1, wherein the coating further comprises a conductive polymer, an anti-fouling polymer, or a combination thereof.

12. (canceled)

13. (canceled)

14. The electrochemical sensor of claim 11, wherein the conductive polymer is sulfonated tetrafluoroethylene (Nafion™).

15. (canceled)

16. (canceled)

17. (canceled)

18. The electrochemical sensor of claim 1, wherein the coating further comprises a nanoparticle.

19. The electrochemical sensor of claim 16, wherein the nanoparticle comprises graphene, carbon nanotubes, borophene, gold, or platinum.

20. (canceled)

21. The electrochemical sensor of claim 1, wherein the coating is less than about 1.5 mm thick.

22. (canceled)

23. (canceled)

24. The electrochemical sensor of claim 1, wherein the one or more electrodes comprises a counter electrode, a working electrode, a reference electrode, or a combination thereof.

25. The electrochemical sensor of claim 24, wherein the counter electrode comprises platinum and carbon.

26. The electrochemical sensor of claim 24, wherein the working electrode comprises gold and carbon.

27. The electrochemical sensor of claim 24, wherein the reference electrode comprises silver (Ag), silver/silver chloride (Ag/AgCl), or saturated calomel electrode (SCE).

28. The electrochemical sensor of claim 1, wherein the sensor is an amperometric sensor and/or a voltammetric sensor.

29. The electrochemical sensor of claim 1, wherein the electrochemical sensor can detect glucose in a fluid sample.

30.-33. (canceled)

34. The electrochemical sensor of claim 1, wherein the coating allows partitioning of glucose directly from a fluid sample, partitions a biocompatible interface between the sensor and the fluid sample, prevents electrode fouling, and/or provides selectivity for glucose.

35. The electrochemical sensor of claim 1, wherein the electrodes are present in a microfluidic device in communication with a microfluid channel.

36. (canceled)

37. The electrochemical sensor of claim 1, wherein the sensor is miniaturized.

38. A microfluidic device comprising the electrochemical sensor of claim 1.

39. A method for electrochemical detection of glucose in a sample comprising:
exposing a fluid sample obtained from a subject to the electrochemical sensor of claim 1, and
detecting the current generated from the oxidation of H₂O₂ during said exposing, wherein current corresponds to the concentration of glucose in the fluid sample.

40.-42. (canceled)

43. A method of treating a subject afflicted with or at risk of diabetes, the method comprising:
exposing a fluid sample obtained from a subject to the electrochemical sensor of claim 1,
detecting the current generated from the oxidation of H₂O₂ during said exposing, wherein current corresponds to the concentration of glucose in the fluid sample,
and treating the subject for diabetes if the subject has a fasting blood glucose concentration is greater than 100 mg/dl.

44. (canceled)

45. A method of diagnosing a subject with or at risk of diabetes, the method comprising:
exposing a fluid sample obtained from a subject to the electrochemical sensor of claim 1,
detecting the current generated from the oxidation of H₂O₂ during said exposing, wherein current corresponds to the concentration of glucose in the fluid sample,
and diagnosing the subject as having or at risk of having diabetes if the subject has a fasting blood glucose concentration is greater than 100 mg/dl.

46. (canceled)

47. A method of monitoring a subject having or at risk of having diabetes, the method comprising:
exposing a fluid sample obtained from a subject to the electrochemical sensor of claim 1,
detecting the current generated from the oxidation of H₂O₂ during said exposing, wherein current correlates to the concentration of glucose in the fluid sample, thereby monitoring the subject having or at risk of having diabetes.

48.-50. (canceled)
